SEQUENCE LISTING (96 sequences)

SEQ ID NO: 1 - DNA, 22 bp, Artificial Sequence
  mouse Rplp0 - Forward Primer
gatgggcaactgtacctgactg

SEQ ID NO: 2 - DNA, 19 bp, Artificial Sequence
  mouse Rplp0 - Reverse Primer
ctgggctcctcttggaatg

SEQ ID NO: 3 - DNA, 20 bp, Artificial Sequence
  mouse Hprt1 - Forward Primer
cagtcccagcgtcgtgatta

SEQ ID NO: 4 - DNA, 20 bp, Artificial Sequence
  mouse Hprt1 - Reverse Primer
ggcctcccatctccttcatg

SEQ ID NO: 5 - DNA, 21 bp, Artificial Sequence
  mouse Tubulin(H/m) - Forward Primer
cgtgttcggccagagtggtgc

SEQ ID NO: 6 - DNA, 21 bp, Artificial Sequence
  mouse Tubulin(H/m) - Reverse Primer
gggtgagggcatgacgctgaa

SEQ ID NO: 7 - DNA, 22 bp, Artificial Sequence
  mouse Gapdh - Forward Primer
ccttcattgacctcaactacat

SEQ ID NO: 8 - DNA, 20 bp, Artificial Sequence
  mouse Gapdh - Reverse Primer
caaagttgtcatggatgacc

SEQ ID NO: 9 - DNA, 21 bp, Artificial Sequence
  mouse ACTB - Forward Primer
gtgggccgctctaggcaccaa

SEQ ID NO: 10 - DNA, 23 bp, Artificial Sequence
  mouse ACTB - Reverse Primer
ctctttgatgtcacgcacgattt

SEQ ID NO: 11 - DNA, 20 bp, Artificial Sequence
  mouse Tet1 - Forward Primer
aagaagaggaaatgcgaggt

SEQ ID NO: 12 - DNA, 20 bp, Artificial Sequence
  mouse Tet1 - Reverse Primer
ggccatttactggtttgttg

SEQ ID NO: 13 - DNA, 20 bp, Artificial Sequence
  mouse Tet2 - Forward Primer
agcaagagattccgaaggat

SEQ ID NO: 14 - DNA, 20 bp, Artificial Sequence
  mouse Tet2 - Reverse Primer
agtggaggactgagtgcaag

SEQ ID NO: 15 - DNA, 23 bp, Artificial Sequence
  mouse Tet3 - Forward Primer
tgcgattgtgtcgaacaaatagt

SEQ ID NO: 16 - DNA, 21 bp, Artificial Sequence
  mouse Tet3 - Reverse Primer
tccataccgatcctccatgag

SEQ ID NO: 17 - DNA, 20 bp, Artificial Sequence
  mouse Tgfb1 - Forward Primer
ggaatacagggctttcgatt

SEQ ID NO: 18 - DNA, 20 bp, Artificial Sequence
  mouse Tgfb1 - Reverse Primer
ctctgtggagctgaagcaat

SEQ ID NO: 19 - DNA, 20 bp, Artificial Sequence
  mouse TgfbR1 - Forward Primer
ggaatacagggctttcgatt

SEQ ID NO: 20 - DNA, 20 bp, Artificial Sequence
  mouse TgfbR1 - Reverse Primer
ctctgtggagctgaagcaat

SEQ ID NO: 21 - DNA, 20 bp, Artificial Sequence
  mouse TgfbR2 - Forward Primer
agcggggaatttacagaatg

SEQ ID NO: 22 - DNA, 20 bp, Artificial Sequence
  mouse TgfbR2 - Reverse Primer
gaggaatgacagcgatgcta

SEQ ID NO: 23 - DNA, 20 bp, Artificial Sequence
  mouse Tsp1 - Forward Primer
aaagccaaagcgcctattta

SEQ ID NO: 24 - DNA, 20 bp, Artificial Sequence
  mouse Tsp1 - Reverse Primer
tggcggtgagttctagtgag

SEQ ID NO: 25 - DNA, 20 bp, Artificial Sequence
  mouse Acta2 - Forward Primer
ctggcctagcaacactgatt

SEQ ID NO: 26 - DNA, 20 bp, Artificial Sequence
  mouse Acta2 - Reverse Primer
gtggaccttcctctgttgaa

SEQ ID NO: 27 - DNA, 20 bp, Artificial Sequence
  mouse Col1a1 - Forward Primer
atgatgctaacgtggttcgt

SEQ ID NO: 28 - DNA, 20 bp, Artificial Sequence
  mouse Col1a1 - Reverse Primer
tggttagggtcgatccagta

SEQ ID NO: 29 - DNA, 20 bp, Artificial Sequence
  mouse Fn1 - Forward Primer
gacagttggtcaccctgttc

SEQ ID NO: 30 - DNA, 20 bp, Artificial Sequence
  mouse Fn1 - Reverse Primer
tgactttcctgctcaaggtc

SEQ ID NO: 31 - DNA, 20 bp, Artificial Sequence
  mouse Timp1 - Forward Primer
ccgcagtgaagagtttctca

SEQ ID NO: 32 - DNA, 20 bp, Artificial Sequence
  mouse Timp1 - Primer Primer
tcactctccagtttgcaagg

SEQ ID NO: 33 - DNA, 20 bp, Artificial Sequence
  human HPRT1 - Forward Primer
gaccagtcaacaggggacat

SEQ ID NO: 34 - DNA, 20 bp, Artificial Sequence
  human HPRT1 - Reverse Primer
cctgaccaaggaaagcaaag

SEQ ID NO: 35 - DNA, 20 bp, Artificial Sequence
  human RPLP0 - Forward Primer
ggcgacctggaagtccaact

SEQ ID NO: 36 - DNA, 20 bp, Artificial Sequence
  human RPLP0 - Reverse Primer
ccatcagcaccacagccttc

SEQ ID NO: 37 - DNA, 21 bp, Artificial Sequence
  human TUBB - Forward Primer
cgtgttcggccagagtggtgc

SEQ ID NO: 38 - DNA, 21 bp, Artificial Sequence
  human TUBB - Reverse Primer
gggtgagggcatgacgctgaa

SEQ ID NO: 39 - DNA, 24 bp, Artificial Sequence
  human b-actin - Forward Primer
atcaagatcattgctcctcctgag

SEQ ID NO: 40 - DNA, 21 bp, Artificial Sequence
  human b-actin - Reverse Primer
ctgcttgctgatccacatctg

SEQ ID NO: 41 - DNA, 22 bp, Artificial Sequence
  human GAPDH - Forward Primer
ctttgtcaagctcatttcctgg

SEQ ID NO: 42 - DNA, 20 bp, Artificial Sequence
  human GAPDH - Reverse Primer
tcttcctcttgtgctcttgc

SEQ ID NO: 43 - DNA, 18 bp, Artificial Sequence
  human U6 - Forward Primer
gctcgcttcggcagcaca

SEQ ID NO: 44 - DNA, 21 bp, Artificial Sequence
  human U6 - Reverse Primer
aacgcttcacgaatttgcgtg

SEQ ID NO: 45 - DNA, 20 bp, Artificial Sequence
  human Lin28B - Forward Primer
aaaacagaacacggaagcag

SEQ ID NO: 46 - DNA, 20 bp, Artificial Sequence
  human Lin28B - Reverse Primer
gtaatggcattgcatgttca

SEQ ID NO: 47 - DNA, 20 bp, Artificial Sequence
  human TET1 - Forward Primer
gcagcgtacaggccaccact

SEQ ID NO: 48 - DNA, 20 bp, Artificial Sequence
  human TET1 - Reverse Primer
agccggtcggccattggaag

SEQ ID NO: 49 - DNA, 23 bp, Artificial Sequence
  human TET2 - Forward Primer
ttcgcagaagcagcagtgaagag

SEQ ID NO: 50 - DNA, 23 bp, Artificial Sequence
  human TET2 - Reverse Primer
agccagagacagcgggattcctt

SEQ ID NO: 51 - DNA, 20 bp, Artificial Sequence
  human TET3 - Forward Primer
gacgagaacatcggcggcgt

SEQ ID NO: 52 - DNA, 20 bp, Artificial Sequence
  human TET3 - Reverse Primer
gtggcagcggttgggcttct

SEQ ID NO: 53 - DNA, 21 bp, Artificial Sequence
  human TGFB1 - Forward Primer
caagcagagtacacacagcat

SEQ ID NO: 54 - DNA, 22 bp, Artificial Sequence
  human TGFB1 - Reverse Primer
tgctccacttttaacttgagcc

SEQ ID NO: 55 - DNA, 20 bp, Artificial Sequence
  human TGFBR2 - Forward Primer
ggttcctgtgtgcccttatt

SEQ ID NO: 56 - DNA, 20 bp, Artificial Sequence
  human TGFBR2 - Reverse Primer
tgcaacccatgaaggtaaaa

SEQ ID NO: 57 - DNA, 20 bp, Artificial Sequence
  human TSP1 - Forward Primer
agcgtcttcaccagagacct

SEQ ID NO: 58 - DNA, 20 bp, Artificial Sequence
  human TSP1 - Reverse Primer
cattcaccacgttgttgtca

SEQ ID NO: 59 - DNA, 20 bp, Artificial Sequence
  human ACTA2 - Forward Primer
agttacgagttgcctgatgg

SEQ ID NO: 60 - DNA, 20 bp, Artificial Sequence
  human ACTA2 - Reverse Primer
gaggtccttcctgatgtcaa

SEQ ID NO: 61 - DNA, 20 bp, Artificial Sequence
  human COL1A1 - Forward Primer
ggacacagaggtttcagtgg

SEQ ID NO: 62 - DNA, 20 bp, Artificial Sequence
  human COL1A1 - Reverse Primer
ccagtagcaccatcatttcc

SEQ ID NO: 63 - DNA, 20 bp, Artificial Sequence
  human FN1 - Forward Primer
agtgggagacctcgagaaga

SEQ ID NO: 64 - DNA, 18 bp, Artificial Sequence
  human FN1 - Reverse Primer
actgtgacagcaggagca

SEQ ID NO: 65 - DNA, 20 bp, Artificial Sequence
  human TIMP1 - Forward Primer
tacttccacaggtcccacaa

SEQ ID NO: 66 - DNA, 20 bp, Artificial Sequence
  human TIMP1 - Reverse Primer
attcctcacagccaacagtg

SEQ ID NO: 67 - DNA, 15 bp, Artificial Sequence
  human TGFB1 - ChIP Forward Primer
cctgccgacccagcc

SEQ ID NO: 68 - DNA, 17 bp, Artificial Sequence
  human TGFB1 - ChIP Reverse Primer
ctcgctgtctggctgct

SEQ ID NO: 69 - DNA, 25 bp, Artificial Sequence
  human TGFBR2 - ChIP Forward Primer
gggctggtctaggaaacatgattgg

SEQ ID NO: 70 - DNA, 24 bp, Artificial Sequence
  human TGFBR2 - ChIP Reverse Primer
gaaacaggaaactcctcgccaaca

SEQ ID NO: 71 - DNA, 20 bp, Artificial Sequence
  human TSP1 - ChIP Forward Primer
gcccattggccggaggaatc

SEQ ID NO: 72 - DNA, 21 bp, Artificial Sequence
  human TSP1 - ChIP Reverse Primer
ctgaactcgcaggccagctcg

SEQ ID NO: 73 - DNA, 21 bp, Artificial Sequence
  human TGFBR2(methylated) - Forward Primer
ggagaagggagaaggttttcg

SEQ ID NO: 74 - DNA, 26 bp, Artificial Sequence
  human TGFBR2(methylated) - Reverse Primer
aataactcactcaacttcaactcaac

SEQ ID NO: 75 - DNA, 21 bp, Artificial Sequence
  human TGFBR2(unmethylated) - Forward Primer
aggagagggagaaggtttttg

SEQ ID NO: 76 - DNA, 26 bp, Artificial Sequence
  human TGFBR2(unmethylated) - Reverse Primer
aataactcactcaacttcaactcaac

SEQ ID NO: 77 - DNA, 17 bp, Artificial Sequence
  human TSP1(methylated) - Forward Primer
cgagtttagggtttttg

SEQ ID NO: 78 - DNA, 27 bp, Artificial Sequence
  human TSP1(methylated) - Reverse Primer
aaaacgacttacctatatataccgaaa

SEQ ID NO: 79 - DNA, 22 bp, Artificial Sequence
  human TSP1(unmethylated) - Forward Primer
tgtgagtttagggtttttgttg

SEQ ID NO: 80 - DNA, 27 bp, Artificial Sequence
  human TSP1(unmethylated) - Reverse Primer
aaaacgacttacctatatataccgaaa

SEQ ID NO: 81 - DNA, 21 bp, Artificial Sequence
  human TGFB1(methylated) - Forward Primer
ggagaagggagaaggttttcg

SEQ ID NO: 82 - DNA, 26 bp, Artificial Sequence
  human TGFB1(methylated) - Reverse Primer
aataactcactcaacttcaactcaac

SEQ ID NO: 83 - DNA, 31 bp, Artificial Sequence
  human TGFB1(unmethylated) - Forward Primer
tttgggttattttttttttattttttttcgc

SEQ ID NO: 84 - DNA, 24 bp, Artificial Sequence
  human TGFB1(unmethylated) - Reverse Primer
actctaaaacctcaaactactcct

SEQ ID NO: 85 - DNA, 21 bp, Artificial Sequence
  human TET3 siRNA sense sequence
cagcaacuccuagaacugatt

SEQ ID NO: 86 - DNA, 21 bp, Artificial Sequence
  human TET3 siRNA antisense sequence
ucaguucuaggaguugcugga

SEQ ID NO: 87 - DNA, 29 bp, Artificial Sequence
  AAV-siTET3
cggtaccatctcctatttctcagagggag

SEQ ID NO: 88 - DNA, 29 bp, Artificial Sequence
  AAV-siP2
cctttgctgctgtgtgtgggcccctgctc

SEQ ID NO: 89 - DNA, 29 bp, Artificial Sequence
  AAV-sihTET3
gcccacaaggaccagcataacctctacaa

SEQ ID NO: 90 - DNA, 29 bp, Artificial Sequence
  AAV-sihP2
atggtcagcgtgaacgcgcccctcggggc

SEQ ID NO: 91 - DNA, 218 bp, Artificial Sequence
  mouse P2 promoter DMR sequence
tgaggtctagccaggttgctgttgccatgacaaaagcatgacaatccccagccacaggttccctaagtgactggttactgtttaacgtatccacccaccttgggtgattagaagaatcaataagataaccgggcggggtggcagctggctgtgctcactgccttcctggtggactggctcccggtggcccctttgctgctgtgtgtgggcccctgctc

SEQ ID NO: 92 - DNA, 219 bp, Artificial Sequence
  TGFB1 - CTRR Sequence
cctgccgacccagccggtccccgccgccgccgcccttcgcgccctgggccatctccctcccacctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagt

SEQ ID NO: 93 - DNA, 231 bp, Artificial Sequence
  TSP1 - CTRR Sequence (-69)
cccattggccggaggaatccccaggaatgcgagcgcccctttaaaagcgcgcggctcctccgccttgccagccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggtaagtcgcccc

SEQ ID NO: 94 - DNA, 159 bp, Artificial Sequence
  TGFBR2 - CTRR Sequence
gggctggtctaggaaacatgattggcagctacgagagagctaggggctggacgtcgaggagagggagaaggctctcgggcggagagaggtcctgcccagctgttggcgaggagtttcctgtttcccccgcagcgctgagttgaagttgagtgagtcact

SEQ ID NO: 95 - DNA, 238 bp, Artificial Sequence
  MED12 - CTRR Sequence
aacgtgcccccttgttgtctctcggccgccgtcctctcaaccaccgccccccttttcggctccctctcccccttcccgttcccccagtcagcctggccctgctggtgcctccggcgctacgggctgggcaagatggcggccttcgggatcttgagctacgaacaccggcccctgaagcggccgcggctggggcctcccgatgtttaccctcaggaccccaaacagaaggaggtgcgtt

SEQ ID NO: 96 - DNA, 232 bp, Artificial Sequence
  TSP1 - CTRR Sequence (-70)
gcccattggccggaggaatccccaggaatgcgagcgcccctttaaaagcgcgcggctcctccgccttgccagccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggtaagtcgcccc

(19) United States
(12) Patent Application Publication
(10) Pub. No.: US 2022/0160751 A1
(43) Pub. Date: May 26, 2022

(54) METHODS OF TREATMENT OF DISEASES AND DISORDERS ASSOCIATED WITH INCREASED TET LEVEL, INCREASED H19 LEVEL, INCREASED LEVEL OF TGF SIGNALING, OR ANY COMBINATION THEREOF

(71) Applicant: YALE UNIVERSITY, New Haven, CT (US)

(72) Inventor: Yingqun Huang, East Haven, CT (US)

(21) Appl. No.: 17/600,984

(22) PCT Filed: Apr. 2, 2020
(86) PCT No.: PCT/US20/26290

§ 371 Date: Oct. 1, 2021

Related U.S. Application Data
(60) Provisional application No. 62/827,938, filed on Apr. 2, 2019.

(51) Int. Cl. A61K 31/7105; A61K 35/76; A61K 45/06

(52) U.S. Cl. CPC A61K 31/7105; A61K 45/06; A61K 35/76

(57) ABSTRACT

The present invention relates to H19, TET, TGF, HNF, or isoforms thereof, as novel pharmacological targets for the treatment of diseases or disorders, such as cancer, fibrosis, and diabetes, associated with increased TET level, increased H19 level, increased TGF level, or any combination thereof.

### STATEMENT REGARDING FEDERALLY SPONSORED RESEARCH OR DEVELOPMENT

This invention was made with government support under DK119386 awarded by the National Institutes of Health. The government has certain rights in the invention.

### BACKGROUND OF THE INVENTION

Liver fibrosis, characterized by a progressive, pathological accumulation of extracellular matrix (ECM) proteins, is a major cause of morbidity and mortality worldwide. Fibrosis develops when the liver is injured due to chronic liver diseases, such as viral hepatitis, alcoholic fatty liver disease, and obesity/type 2 diabetes-associated nonalcoholic fatty liver disease (NAFLD). So far, effective treatments to halt or reverse fibrosis are lacking. Understanding the signal transduction pathways responsible for the development and progression of fibrosis is key to prevention and cure.

A critical event in fibrogenesis is activation of hepatic stellate cells (HSCs) which are the primary source of ECM proteins (Li Y et al., 2017, Hepatol Res 47:186-193; Tsuchida T et al., 2017, Nat Rev Gastroenterol Hepatol 14:397-411). Quiescent HSCs function to store and metabolize vitamin A and to control ECM turnover by releasing limited amounts of ECM molecules, matrix metalloproteinases and their inhibitors. Liver injury activates and transforms HSCs into fibrogenic myofibroblasts, a unique population of mesenchymal cells that acquire the ability to produce α smooth muscle actin (α-SMA) and abundant ECM components including type I collagen (COL1A1), fibronectin (FN1), and ECM remodeling enzymes, such as thrombospondin 1 (TSP1) and inhibitor of metalloproteinases-1 (TIMP1). As HSC activation represents the major source of fibrogenic myofibroblasts irrespective of the underlying cause of liver damage (Tsuchida T et al., 2017, Nat Rev Gastroenterol Hepatol 14:397-411; Henderson N C et al., 2013. Nat Med 19:1617-1624), HSCs have emerged as a key therapeutic target in liver fibrosis (Tsuchida T et al., 2017, Nat Rev Gastroenterol Hepatol 14:397-411; Higashi T et al., 2017, Adv Drug Deliv Rev. 121:27-42).

On the other hand, uterine fibroids (UFs) or leiomyomas are benign tumors arising from the smooth muscle compartment of the uterus. Up to 60% of reproductive-aged women and 80% of all women suffer from fibroids during their lifetime (Laughlin S K et al., 2010, Semin Reprod Med 28:204-217). Fibroids are associated with pelvic pain, excessive bleeding, infertility, and pregnancy complications, and they are the leading indication for hysterectomy. Despite the high prevalence and being huge heath and economic burdens, few effective treatment options exist for UFs. This is largely due to the limited understanding of the molecular basis underlying the etiology and pathogenesis of the disease.

UFs are characterized by hyperplastic smooth muscle cells and excessive deposition of extracellular matrix (ECM)(Stewart E A et al., 2016, Nat Rev Dis Primers 2:16043; Styer A K et al., 2016, Best Pract Res Clin Obstet Gynaecol 34:3-12). Many factors contribute to the development of fibroids, including steroid hormones, growth factors, and genetic traits. Mutations in mediator complex subunit 12 (MED12) have been frequently found in fibroids (Makinen N et al., 2011, Science 334:252-255). A gain-of-function mutation of a common MED12 variant was able to drive fibroid formation and cause genomic instability in mice, suggesting a causative role of MED12 mutations in fibroids (Mittal P et al., 2015, J Clin Invest 125:3280-3284). Further, overexpression of wild-type MED12 promotes proliferation of leiomyoma cells (Al-Hendy A et al., 2017, Endocrinology 158:592-603). Other factors that have been implicated in fibroids include high mobility group AT-hook 2 (HMGA2), TGF-β receptor 2 (TGFBR2), thrombospondin 1 (TSP1), Rho GTPase activating protein 26 (ARHGAP26, also called GRAF1), secreted protein acidic and rich in cysteine (SPARC), and Ten eleven translocation (TET) family proteins. While MED12 and HMGA2 have been implicated in smooth muscle hyperplasia, TGFBR2, TSP1, GRAF1, and SPARC are associated with abnormal ECM remodeling (Styer A K et al., 2016, Best Pract Res Clin Obstet Gynaecol 34:3-12; Bogusiewicz Metal., 2012, Histol Histopathol 27:1495-1502; Borahay M A et al., 2015, Mol Med 21:242-256; Aissani B et al., 2015, Fertil Steril 103:528-534 e513; Wong S L et al., 2017, J Pharmacol 174:3-14).

The transforming growth factor β (TGF-β) is the master regulator of fibrosis (Meng X M et al., 2016, Nat Rev Nephrol 12:325-338; Murphy-Ullrich J E et al., 2018, Matrix Biol 68-69:28-43). The canonical TGF-β signaling components include TGF-β ligands (TGF-β1, TGF-β2, and TGF-β3), TGF-β receptor 2 (TGFBR2), TGF-β receptor 1 (TGFBR1), and Smad proteins (Smad2, Smad3, and Smad4). TGF-β isoforms are secreted as latent precursors that need to be converted into biologically active forms by a variety of mechanisms, including proteolytic cleavage by TSP1, in a cell, tissue, and/or disease-specific manner. Normally, only a small fraction of TGF-β is biologically active. Activated TGF-β binds to TGFBR2, which recruits and activates TGFBR1. TGFBR1 then phosphorylates Smad2 and Smad3, which form complexes with Smad4 and translocate into the nucleus to drive transcription of profibrotic molecules, such as α-SMA, COL1A1, FN1, and TIMP1, whereby inducing myofibroblast activation and ECM deposition. Thus, pathological activation of TGF-β signaling plays a critical role in the development and progression of fibrosis (Bogusiewicz M et al., 2012, Histol Histopathol 27:1495-1502; Meng X M et al., 2016, Nat Rev Nephrol 12:325-338; Murphy-Ullrich J E et al., 2018, Matrix Biol 68-69:28-43). A pathological increase in TGF-β signaling is central to activation of HSCs, which themselves are the significant source of TGF-β1 and TSP1 (Breitkopf K et al., 2005, Gut 54:673-681). TSP1 acts as a primary regulator of TGF-β1 bioactivity in a number of fibrotic diseases including diabetic nephropathy, cardiomyopathy, and liver fibrosis (Li Y et al., 2017, Hepatol Res 47:186-193; Murphy-Ullrich J E et al., 2018, Matrix Biol 68-69:28-43). In addition to TSP1, TIMP1 has been shown to increase in hepatic fibrosis both in mouse models and in human tissue samples. While transgenic TIMP1 overexpression promoted hepatic fibrosis in a carbon tetrachloride (CCl4)-induced liver fibrosis mouse model (Yoshiji H et al., 2000, Hepatology 32:1248-1254), blocking TSP1-mediated TGF-β activation by a TSP1 antagonistic peptide attenuated dimethyl-nitrosamine induced liver damage and fibrosis (Li Y et al., 2017, Hepatol Res 47:186-193; Murphy-Ullrich J E et al., 2018, Matrix Biol 68-69:28-43). Taken together, these findings highlight the importance of TSP1 and TIMP1 in the genesis of liver fibrosis.

The Ten-Eleven Translocation (TET) proteins (TET1, TET2, and TET3) belong to a novel class of DNA demethylases that oxidize 5-methylcytosine (5mC) to generate 5-hydroxymethylcytosine (5hmC), which is subsequently converted to unmethylated cytosine (Liu R et al., 2013, Circulation 128:2047-2057; Rasmussen K D et al., 2016, Genes Dev 30:733-750; An J et al., 2017, Exp Mol Med 49:e323). The enzymatic activity of TETs is regulated by co-factors including α-ketoglutarate generated through the TCA cycle and vitamin C, and by post-translational modifications (Etchegaray J P et al., 2016, Mol Cell 62:695-711). Importantly, elevated expressions of TET1 and TET3 have been detected in fibroids as compared to matched myometrium. siRNA knockdown of either TET1 or TET3 leads to decreased proliferation of primary leiomyoma cells, suggesting a potentially important role of TETs in the pathogenesis of fibroids (Navarro A et al., 2014, J Clin Endocrinol Metab 99: E2437-244). Recent integrative genome-scale studies of fibroids harboring different genetic alterations, including MED12 mutations and HMGA2 rearrangements, have uncovered fibroid subtype-specific gene expression signatures, with MED12 and HMGA2 being the most common driver genes that together contribute to 80-90% of all fibroids (Mehine M et al., 2016, Proc Natl Acad Sci USA 113:1315-1320).

While much is known about the role of TETs in development and cancer (Liu R et al., 2013, Circulation 128:2047-2057; Rasmussen K D et al., 2016, Genes Dev 30:733-750; An J et al., 2017, Exp Mol Med 49:e323), little is known of their function and mechanism in liver fibrosis, although altered expressions of TETs have been noted in fibrotic liver diseases but via unclear mechanisms (Page A et al., 2016, J Hepatol 64:661-673).

In addition, the evolutionarily conserved H19 long noncoding RNA (lncRNA) is highly expressed in placentas and fetal tissues and is strongly downregulated in most adult tissues (Gabory A et al., 2010, Bioessays 32:473-480). However, H19 expression is aberrantly elevated in fibrotic conditions in multiple organs including liver, lung and kidney (Song Y et al., 2017, Hepatology 66:1183-1196; Lu Q et al., 2018, Inflammation; Xie H et al., 2016, Oncotarget 7:51473-51481). As a multi-functional lncRNA, H19 is polyadenylated and localizes predominantly in the cytoplasm. Previously reports demonstrated that the H19/let-7 axis where H19 acts as a molecular sponge for microRNA let-7, thereby reducing its bioavailability and preventing it from inhibiting target gene expression at the posttranscriptional level (Kallen A N et al., 2013, Mol Cell 52:101-112).

Endothelial-to-mesenchymal transition (EndMT) occurs during normal development and also contributes to the pathogenesis of adult cardiovascular disease (CVD) (Hong et al, 2018, Eur J Cell Biol) including atherosclerosis (Chen et al, 2015, J Clin Invest 125:4514-4528; Evrard et al, 2016, Nat Commun 7:11853; Moonen et al, 2015, Cardiovacs Res 108:377-386), myocardial infarction (Aisagbonhi O et al., 2011, Dis Model Mech 4:469-483), vascular malformation (Maddaluno et al, 2013, Nature 498:492-496), pulmonary hypertension (Piera-Velazquez et al, 2011, Am J Pathol 179:1074-1080), portal hypertension (Kitao et al, 2009, Am J Pathol 175:616-626), vascular graft failure (Chen et al, 2012, Cell Rep 2:1684-1696; Cooley et al, 2014, Sci Transl Med 6:227ra234), and cardiac fibrosis (Jeong et al, 2016, J Am Coll Cardiol 67:1556-1568; Zeisberg E M et al, 2007, Nat Med 13:952-961). Under pathological conditions EndMT drives transdifferentiation of normal endothelial cells (ECs) into mesenchymal cells (e.g., fibroblast-like cells, myofibroblasts, or smooth muscle cells) with enhanced proliferation, migration, and production of extracellular matrix (ECM). EndMT-derived fibroblast-like cells accumulate prominently in atherosclerotic lesions, contributing to regulation of inflammation, ECM deposition and plaque instability (Evrard et al, 2016, Nat Commun 7:11853)

Endothelial TGF-β signaling plays a key role in initiation and progression of EndMT (Chen et al, 2015, J Clin Invest 125:4514-4528; Chen et al, 2012, Cell Rep 2:1684-1696; Cooley et al, 2014, Sci Transl Med 6:227ra234; Evrard et al, 2016, Nat Commun 7:11853; Jimenez et al., 2016, Matrix Biol 51:26-36). The canonical components of the TGF-β signaling pathway include the TGF-β ligand, TGF-β receptor 2 (TGFBR2) and 1 (TGFBR1), and Smad proteins (Smad2, Smad3, and Smad4). TGF-β is secreted in an inactive form that is activated by a variety of mechanisms including proteolytic cleavage by thrombospondin 1 (TSP1) (Murphy-Ullrich J E et al., 2018, Matrix Biol 68-69:28-43). Active TGF-β binds to TGFBR2, which in turn recruits and activates TGFBR1. Activated TGFBR1 phosphorylates Smad2 and Smad3 which then complex with Smad4 and move into the nucleus, where they drive transcription of gene networks promoting epithelial-to-mesenchymal transition (EMT) and EndMT (Jimenez et al., 2016, Matrix Biol 51:26-36; Meng X M et al., 2016, Nat Rev Nephrol 12:325-338; Piera-Velazquez et al, 2016, J Clin Med 5).

Expressed and secreted by vascular ECs and smooth muscle cells (SMCs), TSP1 has been detected in human atherosclerotic plaques and its increased expression has been associated with activated or injured endothelium (DiPietro et al, 1994, J Vasc Res 31:178-185; Kessler et al, 2015, Circulation 131:1191-1201; Reed et al, 1995, Am J Pathol 147:1068-1080). Importantly, intra-arterial delivery of TSP1 antibodies accelerates re-endothelialization and reduces neointima formation in balloon-injured rat carotid arteries (Chen et al, 1999, Circulation 100:849-854). Similarly, Tsp1−/− mice show decreased neointima formation following carotid artery ligation (Moura et al, 2007, Arterioscler Thromb Vasc Biol 27:2163-2169). These findings point to an important role of TSP1 in the pathogenesis of atherosclerosis and restenosis.

TET2 was found to be enriched in mature contractile SMCs in normal vasculatures and functions to maintain SMC phenotype by promoting expression of key pro-contractile genes. Decreased TET2 expression was observed in both human and mouse atherosclerotic lesions (Liu et al, 2013, Circulation 128:2047-2057; Peng et al, 2016, Oncotarget 7:76423-76436; Yang Q et al, 2016, Ann Biomed Eng 44:2218-2227). In contrast, an increase in TET1 expression was detected in human carotid artery atherosclerotic plaques, but the identity of the TET1-expressing cells and the biological significance of TET1 expression are unclear (Greissel et al, 2015, Thromb Haemost 114:390-402).

The evolutionarily conserved H19 gene is developmentally regulated; it is highly expressed in fetal tissues and strongly downregulated after birth, except in a few tissues such as skeletal muscle and heart (Gabory A et al., 2010, Bioessays 32:473-480; Han et al, 1996, J Clin Invest 97:1276-1285). H19 encodes a long noncoding RNA (lncRNA) that is capped and polyadenylated and resides predominantly in the cytoplasm. Prominent H19 expression is detected in both ECs and SMCs of prenatal rabbit aorta but becomes undetectable in adult animals (Han et al, 1996, J Clin Invest 97:1276-1285). However, H19 is re-expressed in rat intima after blood vessel injury (Kim et al, 1994, J Clin Invest 93:355-360), in left ventricle interstitial vascular structures of heart failure human patients (Greco et al, 2016, J Transl Med 14:183), and in human atherosclerotic plaques (Han et al, 1996, J Clin Invest 97:1276-1285). The identities of these H19-expressing cells and the biological significance of H19 expression have remained undefined. Recently, H19 was identified as an important regulator of abdominal aortic aneurysm; H19 expression was upregulated in SMCs in animal abdominal aortic aneurysm models and was demonstrated to induce SMC apoptosis in part by promoting transcription of hypoxia-induced factor 1a (Li et al, 2018, Circulation 138:1551-1568).

Notably, an association between common polymorphisms of H19 and the risk and severity of coronary artery disease (CAD) has been reported (Gao et al, 2015, Mutat Res 772:15-22). Likewise, increased plasma levels of H19 RNA have been linked to increased risks of CAD and heart failure (Zhang Z et al, 2017, Sci Rep 7:7491). Although H19 has been shown to contribute to the pathogenesis of CVD (Hofmann et al, 2018, Cardiovasc Res; Li et al, 2018, Circulation 138:1551-1568), its role in endothelial TGF-β signaling, a key process linked to EndMT and various forms of CVD, has not been documented.

Thus, there is a need in the art for improved treatments of diseases and disorders associated with increased TET, increased H19 level, increased level of TGF signaling, or any combination thereof. The present invention addresses this unmet need in the art.

### SUMMARY OF THE INVENTION

The present invention relates to ten-eleven translocation proteins (TET), H19, transforming growth factor (TGF), hepatocyte nuclear factor (HNF), or isoforms thereof, as novel pharmacological targets for the treatment of diseases or disorders. The present invention also provides methods relating to the pharmacological targets of the invention that can be used to establish and evaluate treatment regimens for the diseases or disorders of the invention.

In one aspect, the invention relates to methods of treating a disease or disorder associated with increased TET level, increased H19 level, increased level of TGF signaling, or any combination thereof. In one embodiment, the method comprises administering a treatment to a subject in need thereof. In various embodiments, the treatment comprises at least one selected from the group consisting of a reduction of TET level or activity, a reduction of H19 level or activity, a reduction of TGF signaling or activity, a reduction of HNF level or activity, a reduction of HNF isoform level or activity, and any combination thereof, in a subject in need thereof.

In various embodiments, the TET is a ten-eleven translocation protein 1 (TET1), ten-eleven translocation protein 2 (TET2), ten-eleven translocation protein 3 (TET3), or any combination thereof.

In various embodiments, the TGF signaling comprises at least one component selected from the group consisting of a transforming growth factor alpha (TGF-α), transforming growth factor alpha (TGF-α) isoform, transforming growth factor beta (TGF-β), transforming growth factor beta (TGF-β) isoform, TGF-β receptor 1 (TGFBR1), TGF-β receptor 2 (TGFBR2), Smad protein 2 (Smad2), Smad protein 3 (Smad3), Smad protein 4 (Smad4), phosphorylated SMAD2 (p-SMAD2), phosphorylated SMAD3 (p-SMAD3), phosphorylated SMAD4 (p-SMAD4), and any combination thereof. In various embodiments, the TGF-β or an isoform thereof, is a transforming growth factor beta 1 (TGF-β1), transforming growth factor beta 2 (TGF-β2), transforming growth factor beta 3 (TGF-β3), transforming growth factor beta 4 (TGF-β4), or any combination thereof.

In various embodiments, the HNF is a hepatocyte nuclear factor 1 alpha (HNF1α), hepatocyte nuclear factor 1 beta (HNF1β), hepatocyte nuclear factor 3 alpha (HNF3α), hepatocyte nuclear factor 3 beta (HNF3β), hepatocyte nuclear factor 4 alpha (HNF4α), hepatocyte nuclear factor 4 gamma (HNF4γ), hepatocyte nuclear factor 6 alpha (HNF6α), hepatocyte nuclear factor 6 beta (HNF6β), or any combination thereof.

In various embodiments, the HNF isoform is a P1-derived hepatocyte nuclear factor 4 alpha (HNF4α P1) isoform, a P2-derived hepatocyte nuclear factor 4 alpha (HNF4α P2) isoform, or any combination thereof. In various embodiments, the HNF4α P1 isoform is a P1-derived hepatocyte nuclear factor 4 alpha 1 (HNF4α1) isoform, P1-derived hepatocyte nuclear factor 4 alpha 2 (HNF4α2) isoform, P1-derived hepatocyte nuclear factor 4 alpha 3 (HNF4α3) isoform, P1-derived hepatocyte nuclear factor 4 alpha 4 (HNF4α4) isoform, P1-derived hepatocyte nuclear factor 4 alpha 5 (HNF4α5) isoform, P1-derived hepatocyte nuclear factor 4 alpha 6 (HNF4α6) isoform, or any combination thereof. In various embodiments, the P2-derived HNF4α P2 isoform is a P2-derived hepatocyte nuclear factor 4 alpha 7 (HNF4α7) isoform, P2-derived hepatocyte nuclear factor 4 alpha 8 (HNF4α8) isoform, P2-derived hepatocyte nuclear factor 4 alpha 9 (HNF4α9) isoform, or any combination thereof.

In various aspects of the invention, the method of treatment comprises administering a therapeutically effective amount of at least one selected from a TET inhibitor, a H19 inhibitor, a TGF signaling inhibitor, a HNF inhibitor, or any combination thereof to a subject in need thereof. In various embodiments, the TET inhibitor is a TET1 inhibitor, a TET2 inhibitor, a TET3 inhibitor, or any combination thereof. In various embodiments, the TET inhibitor is a nucleic acid, a peptide, a small molecule chemical compound, an siRNA, a ribozyme, an antisense nucleic acid, an aptamer, a peptidomimetic, an antibody, an antibody fragment, an induced protein degradation, or any combination thereof.

In various embodiments, the H19 inhibitor is a nucleic acid, a peptide, a small molecule chemical compound, an siRNA, a ribozyme, an antisense nucleic acid, an aptamer, a peptidomimetic, an antibody, an antibody fragment, an induced protein degradation, or any combination thereof.

In various embodiments, the TGF inhibitor is a TGFα inhibitor, a TGFβ1 inhibitor, a TGFβ2 inhibitor, a TGFβ3 inhibitor, a TGFβ4 inhibitor, a TGFBR1 inhibitor, a TGFBR2 inhibitor, a Smad2 inhibitor, a Smad3 inhibitor, a Smad4 inhibitor, or any combination thereof. In various embodiments, the TGF inhibitor is a nucleic acid, a peptide, a small molecule chemical compound, an siRNA, a ribozyme, an antisense nucleic acid, an aptamer, a peptidomimetic, an antibody, an antibody fragment, an induced protein degradation, or any combination thereof.

In various embodiments, the HNF inhibitor is a HNF1α inhibitor, a HNF1β inhibitor, a HNF3α inhibitor, a HNF3β inhibitor, a HNF4α inhibitor, a HNF4γ inhibitor, a HN6α inhibitor, a HNF6β inhibitor, or any combination thereof. In various embodiments, the HNF inhibitor is a nucleic acid, a peptide, a small molecule chemical compound, an siRNA, a ribozyme, an antisense nucleic acid, an aptamer, a peptidomimetic, an antibody, an antibody fragment, an induced protein degradation, or any combination thereof.

In one aspect of the invention, the reduction of TET level comprises an inhibition of TET. In one embodiment, the reduction of TET level comprises a reduction of at least one TET co-factor. In one embodiment, the reduction of TET level comprises a reduction of H19 level or activity. In various embodiments, the TET co-factor is α-ketoglutarate, vitamin C, iron, 2-oxoglutarate, or any combination thereof. In one embodiment, the reduction of TET level comprises a siRNA knockdown of TET. In one embodiment, the siRNA knockdown of TET is a viral-mediated siRNA knockdown of TET. In one embodiment, the viral-mediated siRNA knockdown of TET comprises at least one AAV vector. In one embodiment, the reduction of TET level reduces TGF signaling. In one embodiment, the reduction of TET level blocks TGF signaling. In one embodiment, the siRNA knockdown of TET reduces TGF signaling.

In various aspects of the invention, the treatment further comprises a reduction of estradiol, progesterone, glucagon, MED12, GRAF1, SPARC, PEPCK, G6PC, PGC, PGC-1α, TSP, TSP1, FN1, VIM, COL1A1, COL3A1, COL4A1, COL5A2, HMGA2, SLUG, T1MP1, α-SMA, SMAD2, SMAD3, SMAD4, p-SMAD2, p-SMAD3, p-SMAD4, SM22-α, LIN28B, NOTCH3, collagen 1, fibronectin, or any combination thereof, in a subject in need thereof. In various embodiments, the treatment further comprises reducing an expression of Tet3, Hnf4α, Pck1, G6pc, or any combination thereof, in a subject in need thereof.

In one aspect of the invention, the disease or disorder is a disease or disorder associated with gluconeogenesis regulation. In another aspect of the invention, the disease or disorder is a cancer or fibrosis. In various embodiments, the disease or disorder is a liver fibrosis, hepatic cirrhosis, chronic liver disease, viral hepatitis, alcoholic fatty liver disease, obesity, nonalcoholic fatty liver disease (NAFLD), type 1 diabetes, type 2 diabetes, pulmonary fibrosis, renal fibrosis, cardiac fibrosis, dermal fibrosis, cystic fibrosis, pancreatic fibrosis, esophageal cancer, stomach cancer, pancreatic cancer, liver cancer, gallbladder cancer, biliary track cancer, MALT lymphoma, gastrointestinal stomach tumors, cholangiocarcinoma, colorectal cancer, colon cancer, anal cancer, gastrointestinal carcinoid tumor, neuroendocrine tumors, small bowel cancer, gastrointestinal cancer, melanoma, lung cancer, kidney cancer, uterine fibrosis, leiomyomas, atherosclerosis, coronary artery disease, cardiovascular disease, cardiovascular disease involving endothelial dysfunction, or any combination thereof.

In various embodiments, the treatment comprises an intravenous, oral, aerosol, parenteral, ophthalmic, pulmonary, or topical administration. In one embodiment, the method further comprises administering adjuvant radiotherapy to the subject in need thereof.

In one aspect of the invention, the method of treatment comprises administering a therapeutically effective amount of at least one FOXA2 inhibitor. In various embodiments, the FOXA2 inhibitor is a nucleic acid, a peptide, a small molecule chemical compound, an siRNA, a ribozyme, an antisense nucleic acid, an aptamer, a peptidomimetic, an antibody, an antibody fragment, an induced protein degradation, or any combination thereof.

In another aspect of the invention, the method of treatment comprises administering a therapeutically effective amount of at least one let-7 promoter. In various embodiments, the let-7 promoter is a nucleic acid, a peptide, a small molecule chemical compound, an siRNA, a ribozyme, an antisense nucleic acid, an aptamer, a peptidomimetic, an antibody, an antibody fragment, or any combination thereof.

### BRIEF DESCRIPTION OF THE DRAWINGS

The following detailed description of various embodiments of the invention will be better understood when read in conjunction with the appended drawings. For the purpose of illustrating the invention, there are shown in the drawings illustrative embodiments. It should be understood, however, that the invention is not limited to the precise arrangements and instrumentalities of the embodiments shown in the drawings.

FIG. 1 depicts schematic of the Hepatocyte Nuclear Factor 4α (HNF4α) gene. The gene is composed of 13 exons and contains two promoters, P2 and its downstream P1, that drive expression of 9 isoforms (α1 to α9) via alternative splicing. Antibodies that specifically recognize the N-terminal regions of P2 (orange) and P1 (green) isoforms, respectively, have been well-characterized. siRNAs targeted to the region downstream of P2 and upstream of P1 promoters have been designed to specifically knockdown the P2 isoforms without altering expression of P1 isoforms.

FIG. 2, comprising FIG. 2A through FIG. 2D, depicts glucagon-induced, H19 mediated upregulation of TET3 (Data are representative of two independent experiments and are presented as mean±SEM. *p<0.05, **p<0.01). FIG. 2A depicts Quantitative Polymerase Chain Reaction (qPCR) results of the indicated RNAs in liver tissues isolated from mice fed ad libitium or fasted for 12 h. n=3. FIG. 2B depicts qPCR results of H19 and TET3 RNAs from WT and H19 KO primary hepatocytes treated with vehicle or glucagon (20 nM) for 24 h. n=3. FIG. 2C depicts qPCR results of H19 and TET3 RNAs from mouse primary hepatocytes infected with AAV-vec or AAV-H19 for 48 h. n=3. FIG. 2D depicts qPCR results of TET3 mRNA from liver tissues isolated from mice infected with AA-vec or AAV-H19 for 14 days. n=3.

FIG. 3, comprising FIG. 3A through FIG. 3L, depicts data demonstrating that TET3 promotes HGP (Data depicted in FIG. 3A through FIG. 3F are representative of at least two independent experiments; and data depicted in FIG. 3G through FIG. 3L are representative of two independent experiments. All data are presented as mean±SEM. *p<0.05, **p<0.01). FIG. 3A depicts qPCR results of TET3, PCK1 and G6PC following infection with Ad-GFP or Ad-TET3 for 60 h in primary H19 KO hepatocytes. n=3. FIG. 3B depicts immunoblotting (IB) of TET3, PCK1 and G6PC following infection with Ad-GFP or Ad-TET3 for 60 h in primary H19 KO hepatocytes. n=3. FIG. 3C depicts glucose production by primary H19 KO hepatocytes treated as in FIG. 3A. n=3. FIG. 3D depicts qPCR results of TET3, PCK1 and G6PC following infection with AAV-scr or AAV-siTET3 for 60 h in primary hepatocytes. n=3. FIG. 3E depicts IB of TET3, PCK1 and G6PC following infection with AAV-scr or AAV-siTET3 for 60 h in primary hepatocytes. n=3. FIG. 3F depicts glucose production by primary hepatocytes treated as in FIG. 3D. n=3. FIG. 3G depicts qPCR results of TET3, PCK1 and G6PC from liver tissues isolated from H19 KO mice injected via tail vein with Ad-GFP or Ad-TET3. n=4-6. Liver tissues were collected from ad libitum fed mice on day 10 post infection. FIG. 3H depicts D3 of TET3, PCK1 and G6PC from liver tissues isolated from H19 KO mice injected via tail vein with Ad-GFP or Ad-TET3. n=4-6. Liver tissues were collected from ad libitum fed mice on day 10 post infection. FIG. 3I depicts blood glucose and insulin levels of H19 KO mice treated as in FIG. 3G. n=6. FIG. 3J depicts fasting blood glucose and insulin levels of mice 10 days after infection with AAV-scr or AAV-siTET3. n=5-6. FIG. 3K depicts PTT in mice treated as in FIG. 3J. n=5-6, Two-way ANOVA with Sidak post-test. FIG. 3L depicts IB of TET3, PEPCK and G6PC proteins from liver tissues isolated from mice treated as in FIG. 3J. n=4.

FIG. 4, comprising FIG. 4A through FIG. 4C, depicts qPCR results of TET1 and TET2 (All data are representative of two independent experiments and are presented as mean±SEM. *p<0.05, **p<0.01). FIG. 4A depicts qPCR results of TET1 and TET2 from primary mouse hepatocytes infected with AAV-scr or AAV-siTET3. n=3. FIG. 4A is related to FIG. 3D and FIG. 3E. FIG. 4B depicts hepatic TET3 expression based on dataset available from the Gene Expression Omnibus (accession number GSE15653). n=4-9. FIG. 4C depicts qPCR results of TET3, TET2, and TET1 mRNAs in human primary hepatocytes infected with AAV-scr or AAV-sihTET3 for 48 h and treated with vehicle or glucagon (20 nM) for 24 h. n=3, One-way ANOVA with Tukey post-test. FIG. 4C is related to FIG. 6I.

FIG. 5, comprising FIG. 5A through FIG. 5P, depicts TET3 expression positively correlates with P2 isoform expression (All data are representative of at least two independent experiments and are presented as mean±SEM. *p<0.05, **p<0.01). FIG. 5A depicts IB of TET3 from liver tissues isolated from mice infected with AAV-vec/AAV-H19. n=3-4. FIG. 5B depicts IB of TET3 from liver tissues isolated from mice ad libitium fed/fasted. n=3-4. FIG. 5C depicts IB of TET3 from liver tissues isolated from mice exposed to normal chow (NC)/HFD for 11 days. n=3-4. FIG. 5D depicts D3 of TET3 from liver tissues isolated from mice exposed to normal chow (NC)/HFD for 12 weeks. n=3-4. FIG. 5E depicts qPCR results of HNF4α P2 and P1 isoforms in liver tissues from mice infected with AAV-vec/AAV-H19. n=4. FIG. 5F depicts qPCR results of HNF4α P2 and P1 isoforms in liver tissues from mice ad libitium fed/fasted. n=4. FIG. 5G depicts qPCR results of HNF4α P2 and P1 isoforms in liver tissues from mice exposed to normal chow (NC)/HFD for 11 days. n=4.

FIG. 5H depicts qPCR results of HNF4α P2 and P1 isoforms in liver tissues from mice exposed to normal chow (NC)/HFD for 12 weeks. n=4. FIG. 5I depicts D3 of HNF4α P2 and P1 proteins in liver tissues from mice infected with AAV-vec/AAV-H19. n=4. FIG. 5J depicts IB of HNF4α P2 and P1 proteins in liver tissues from mice ad libitium fed/fasted. n=4. FIG. 5K depicts D3 of HNF4α P2 and P1 proteins in liver tissues from mice exposed to normal chow (NC)/HFD for 11 days. n=4. FIG. 5L depicts IB of HNF4α P2 and P1 proteins in liver tissues from mice exposed to normal chow (NC)/HFD for 12 weeks. n=4. FIG. 5M depicts qPCR results of HNF4α P2 and P1 isoforms in liver tissues from H19 KO mice infected with Ad-GFP or Ad-TET3 for 10 days. n=4. FIG. 5N depicts IB of HNF4α P2 and P1 isoforms in liver tissues from H19 KO mice infected with Ad-GFP or Ad-TET3 for 10 days. n=4. FIG. 5O depicts qPCR results of HNF4α P2 and P1 isoforms in liver tissues from mice infected with AAV-scr or AAV-siTET3 for 10 days. n=4. FIG. 5P depicts IB of HNF4α P2 and P1 isoforms in liver tissues from mice infected with AAV-scr or AAV-siTET3 for 10 days. n=4.

FIG. 6, comprising FIG. 6A through FIG. 6J, depicts the mechanism of TET3-induced P2 isoform required for HGP is conserved (Data depicted in FIG. 6A through FIG. 6E are representative of at least two independent experiments; and data depicted in FIG. 6F through FIG. 6J are representative of two independent experiments. All data are presented as mean±SEM. *p<0.05, **p<0.01). FIG. 6A depicts qPCR results of HNF4α P2 and P1 isoforms from WT and H19 KO hepatocytes treated with glucagon (20 nM) or vehicle for 24 h. The ratio of P2-to-P1 isoforms is arbitrarily set as 1 in vehicle treated group. n=3. FIG. 6B depicts qPCR results of HNF4α P2 and P1 isoforms from WT and H19 KO hepatocytes infected with AAV-Vec, AAV-H19, or Ad-TET3 for 48 h. n=3, One-way ANOVA with Dunnett post-test. FIG. 6C depicts qPCR results of HNF4α P2 and P1 isoforms from mouse primary hepatocytes infected with AAV-scr or AAV-siTET3 for 48 h and treated with vehicle or glucagon (20 nM) for 24 h. n=3, One-way ANOVA with Tukey post-test. FIG. 6D depicts qPCR results of HNF4α P2 and P1 isoforms and PCK1 and G6PC mRNAs from mouse primary hepatocytes infected with AAV-scr or AAV-siP2 for 48 h and treated with vehicle or glucagon (20 nM) for 24 h. n=3, One-way ANOVA with Tukey post-test. FIG. 6E depicts glucose production from primary hepatocytes infected with AAV-scr or AAV-siP for 60 h and treated with vehicle or glucagon (20 nM) for 36 h. n=3, One-way ANOVA with Tukey post-test. FIG. 6F depicts fasting blood glucose and insulin levels of mice infected with AAV-scr or AAV-siP2 for 10 days. n=5-6. FIG. 6G depicts PTT in mice treated as in FIG. 6F. n=5-6, Two-way ANOVA with Sidak post-test. FIG. 6H depicts IB of HNF4α P2 and P1 and PEPCK and G6PC proteins from liver tissues isolated from mice treated as in FIG. 6F. n=4. FIG. 6I depicts qPCR results of HNF4α P2 and P1 and PCK1 and G6PC mRNAs in primary human hepatocytes infected with AAV-scr, sihTET3, or sihP2 for 48 h and treated with glucagon (20 nM) or vehicle for 24 h. n=3, One-way ANOVA with Tukey post-test. FIG. 6J depicts glucose production from primary human hepatocytes treated as in FIG. 6I. n=3, One-way ANOVA with Tukey post-test.

FIG. 7, comprising FIG. 7A through FIG. 7I, depicts the P2 isoform epigenetically induced by TET3 contributes to increased HGP in HFD mice (Data depicted in FIG. 7B through FIG. 7E are representative of at least two independent experiments; and data depicted in FIG. 7F through FIG. 7H are representative of two independent experiments. All data are presented as mean±SEM. *p<0.05, **p<0.01. AUC, area under the curve). FIG. 7A depicts DMR sequence in the P2 promoter of mouse Hnf4a (FIG. 7A, left). The three differentially methylated CpGs are highlighted. Numbers mark the starting and ending positions of nucleotides in the chromosome. FIG. 7A also depicts schematic of mouse Hnf4a showing TET3 (red) and RNAP (green) ChIP regions, respectively (FIG. 7A, right). Numbers mark the starting and ending positions of nucleotides in the chromosome. Not drawn to scale. FIG. 7B depicts mouse primary hepatocytes were treated with vehicle (−) or 20 nM of glucagon (+) for 24 h, followed by ChIP-qPCR of P2 and P1 promoters. Neg, negative control region. Data are presented as % input, with gray bars representing background IgG signal. n=3. FIG. 7C depicts mouse hepatocytes were treated as in b, followed by QMSP of P2 (FIG. 7C, left). FIG. 7C also depicts H19 KO hepatocytes were infected with Ad-GFP or Ad-TET3 for 48 h, followed by QMSP of P2 (FIG. 7C, right). n=3. FIG. 7D depicts mouse hepatocytes were treated with vehicle or glucagon (20 nM) for 24 h, followed by RNAP ChIP of P2 and P1 (FIG. 7D, left). FIG. 7D also depicts H19 KO mouse hepatocytes were infected with Ad-GFP or Ad-TET3 for 48 h, followed by RNAP ChIP (FIG. 7D, right). n=3. FIG. 7E depicts U-2 OS cells were transfected with plasmids expressing human HNF4α8 or HNF4α2, together with gAF1, a Renilla luciferase reporter, and increasing amounts of a PGC-1a expression vector. Luciferase reporter levels were measured 24 h later. n=3, Two-way ANOVA with Sidak post-test. FIG. 7F depicts fasting blood glucose and insulin levels (Two-tailed Student's t tests), and PTT, GTT, and ITT in HFD mice infected with AAV-scr or AAV-siTET3 for 10 days. n=7-8, Two-way ANOVA with Sidak post-test. FIG. 7G depicts fasting blood glucose and insulin levels (Two-tailed Student's t tests), and PTT, GTT, and ITT in HFD mice infected with AAV-scr or AAV-siP2 for 10 days. n=6-7, Two-way ANOVA with Sidak post-test. FIG. 7H depicts D3 of TET3, HNF4α P2 and P1 isoforms, and PEPCK and G6PC in liver tissues from HFD mice infected with AAV-scr, AAV-siTET3, or AAV-siP2 for 10 days. n=4. FIG. 7I depicts a proposed model.

FIG. 8, comprising FIG. 8A through FIG. 8E, depicts data for mice infected with AAV-scr, AAV-siTET3, or AAV-siP2 for 10 days (Data are representative of two independent experiments and are presented as mean±SEM. #p<0.05, *p<0.05, **p<0.01. AUC, area under the curve). FIG. 8A depicts fasting blood glucose in ob/ob mice infected with AAV-scr, AAV-siTET3, or AAV-siP2 for 10 days. n=5, One-way ANOVA with Dunnett post-test. FIG. 8B depicts PTT in ob/ob mice treated as in FIG. 8A. * AAV-siTET3 vs. AAV-scr, #AAV-siP2 vs. AAV-scr. n=5, Two-way ANOVA with Sidak post-test. FIG. 8C depicts GTT in ob/ob mice treated as in FIG. 8A. n=5, Two-way ANOVA with Sidak post-test. FIG. 8D depicts IB of TET3, HNF4α P2 and P1 isoforms, and PEPCK and G6PC proteins of liver tissues isolated from mice treated as in FIG. 8A. n=4, One-way ANOVA with Dunnett post-test. FIG. 8E depicts qPCR results of HNF4α P2 and P1 isoforms in pancreas tissues isolated from HFD mice infected with AAV-scr or AAV-siP2 for 10 days. n=4

FIG. 9, comprising FIG. 9A through FIG. 9H, depicts TET3 positively regulates expression of key TGF-β signaling genes (All data are presented as mean±SEM. **p<0.01, *p<0.05, ns, not statistically significant. Data depicted in FIG. 9E through FIG. 9H are representative of at least two independent transfection experiments). FIG. 9A depicts qPCR results of TET3, TET2, and TET1 from non-fibrotic (n=5) and fibrotic (n=7) human liver tissues. FIG. 9B depicts representative images of Masson's trichrome (Masson) and hematoxylin-eosin (H&E) staining on liver sections from mice treated with vehicle or CCl4. Magnifications of 4× and 20× are presented. Scale bar, 100 FIG. 9C depicts qPCR results of indicated genes from liver tissues isolated from mice treated with vehicle or CCl4. n=3. RNA extracted from liver tissues pooled from 6 animals per group was analyzed. FIG. 9D depicts D3 of indicated proteins from liver tissues isolated from mice treated with vehicle or CCl4. n=3. Protein extracted from liver tissues pooled from 6 animals per group was analyzed. FIG. 9E depicts qPCR results of indicated genes from LX-2 cells transfected with control siRNA (siCon) or TET3-specific siRNA (siTET3). n=3. RNA and protein were isolated at 48 h post-transfection. FIG. 9F depicts D3 of indicated genes from LX-2 cells transfected with control siRNA (siCon) or TET3-specific siRNA (siTET3). n=3. RNA and protein were isolated at 48 h post-transfection. FIG. 9G depicts qPCR results of indicated genes from LX-2 cells transfected with empty vector (Vec) or a plasmid DNA expressing human TET3 (pTET3). RNA and protein were isolated at 48 h post-transfection. FIG. 9H depicts D3 of indicated genes from LX-2 cells transfected with empty vector (Vec) or a plasmid DNA expressing human TET3 (pTET3). RNA and protein were isolated at 48 h post-transfection.

FIG. 10, comprising FIG. 10A through FIG. 10D, depicts TET3 affects promoter methylation and histone marks of TGFB1, TSP1 and TGFBR2 (All data are presented as mean±SEM. **p<0.01, *p<0.05, ns, not statistically significant. Data depicted in FIG. 10A, FIG. 10C, and FIG. 10D are representative of two independent experiments). FIG. 10A depicts LX-2 cells were transfected with siCon or siTET3, ChIP-qPCR analysis was performed 48 h later. Data are presented as mean relative TET3 enrichment over input after normalization against preimmune IgG background signals. n=3. Blue numbers indicate nucleotide positions relative to the transcriptional start sites, with PCR products depicted as red stripped bars above. FIG. 10B depicts sequences of critical transcription regulatory regions (CTRR) of TGFB1, TSP1 and TGFBR2. The differentially methylated cytosine residues are highlighted in green. The blue numbers mark the positions of the indicated nucleotides relative to the transcriptional start sites. FIG. 10C depicts LX-2 cells were transfected with siCon or siTET3 for 48 h, followed by QMSP analysis. Relative promoter methylations of the indicated genes are shown. n=3. FIG. 10D depicts LX-2 cells were transfected with siCon or siTET3, followed by ChIP-qPCR analysis at 48 h post-transfection. n=3.

FIG. 11, comprising FIG. 11A and FIG. 11B, depicts TGF-β1 promotes TET3 expression and TGF-β signaling in a TET3-dependent manner. LX-2 cells were transfected with siCon or siTET3. Twenty-four hours later, cells were treated with TGF-β1 (5 ng/mL) (+) or vehicle (−), followed by RNA and protein extraction 24 h later (Data are representative of two independent experiments. All data are presented as mean±SEM. **p<0.01, *p<0.05, ns, not statistically significant). FIG. 11A depicts expression of indicated genes that were analyzed by qPCR. n=3, One-way ANOVA with Tukey post-test. FIG. 11B depicts expression of indicated genes that were analyzed by IB. n=3, One-way ANOVA with Tukey post-test.

FIG. 12, comprising FIG. 12A through FIG. 12J, depicts the LIN28B/let-7 axis mediates TGF-β1 induced upregulation of TET3 in HSCs (All data are representative of two independent experiments and presented as mean±SEM. **p<0.01, *p<0.05, ns, not statistically significant). FIG. 12A depicts qPCR results of LIN28B, Let-7a, Let-7b and TET3 from LX-2 cells stimulated with TGF-β1 (5 ng/mL) (+) or vehicle (−). RNAs were extracted at 24 h post-treatment. n=3. FIG. 12B depicts IB of phosphorylated Smads (pSmad3), total Smad3, LIN28B and TET3 from LX-2 cells stimulated with TGF-β1 (5 ng/mL) (+) or vehicle (−). n=3. FIG. 12C depicts schematics of human and mouse TET3 mRNAs, with numbers on top depicting positions of putative let-7-binding sites relative to the transcriptional start sites. Not drawn to scale. FIG. 12D depicts qPCR results of TET3 from LX-2 cells transfected with control miRNA (Con), let-7 mimic (Let-7), or let-7 inhibitor (iLet7). RNA and protein were isolated at 24 h and 48 h post-transfection, respectively. n=3, One-way ANOVA with Tukey post-test. FIG. 12E depicts IB of TET3 from LX-2 cells transfected with control miRNA (Con), let-7 mimic (Let-7), or let-7 inhibitor (iLet7). RNA and protein were isolated at 24 h and 48 h post-transfection, respectively. n=3, One-way ANOVA with Tukey post-test. FIG. 12F depicts qPCR results of indicated genes from primary human HSCs stimulated with TGF-β1 (2 ng/mL) (+) or vehicle (−) for 24 h. n=3. FIG. 12G depicts qPCR results of indicated genes from non-fibrotic (n=5) and fibrotic (n=7) human liver tissues. FIG. 12H depicts representative images of IHC of liver tissue sections from patients with non-fibrotic and fibrotic disease co-stained for α-SMA (pink) and TGF-β1 (brown) or α-SMA (pink) and TET3 (brown), with nuclei stained in blue. Red arrowheads identify activated HSCs. Magnification, 20×; Scale bar, 100 μm. FIG. 12I depicts numbers of activated HSCs (aα-SMA+) co-expressing TGF-β1 (TGF-β1+/α-SMA+, left panel) or TET3 (TET3+/α-SMA+, right panel) per field from liver tissue sections from patients with non-fibrotic (n=3) and fibrotic disease (n=3). FIG. 12J depicts a proposed signal transduction pathway in HSCs.

FIG. 13, comprising FIG. 13A through FIG. 13F, depicts TET3 inhibition attenuates liver fibrosis (One-way ANOVA with Tukey post-test). FIG. 13A depicts representative images for Masson and H&E staining on liver sections from mice treated with vehicle plus AAV-scr (n=6), CCl4 plus AAV-scr (n=6), or CCl4 plus AAV-siTET3 (n=6). Magnification, 4×. FIG. 13B depicts quantification of fibrosis from liver tissue sections from mice treated as in FIG. 13A. Quantification was performed using ImageJ. n=30. FIG. 13C depicts qPCR results of indicated genes from liver tissues isolated from mice treated as in FIG. 13A. RNA and Protein extracted from liver tissues pooled from 6 animals per group was analyzed. n=3. FIG. 13D depicts D3 of indicated genes from liver tissues isolated from mice treated as in FIG. 13A. RNA and Protein extracted from liver tissues pooled from 6 animals per group was analyzed. n=3. FIG. 13E depicts hydroxyproline contents from liver tissues isolated from mice treated as in FIG. 13A. n=6. FIG. 13F depicts serum ALP, ALT and bilirubin from mice treated as in FIG. 13A. n=6.

FIG. 14, comprising FIG. 14A through FIG. 14C, depicts the results that demonstrate that H19 promotes cell proliferation and expression of fibroid-promoting genes. All data are representative of at least two independent experiments and are presented as mean±SEM. *p<0.05, **p<0.01. ns, not statistically significant. FIG. 14A depicts RT-qPCR results of H19 expression in fibroids and matched myometrium (control). n=30. FIG. 14B depicts primary leiomyoma cells derived from fibroid tumors of two patients that were transfected with control siRNA (siCon) or H19-specific siRNA (siH19), followed by RT-qPCR (left panels), cell viability (middle panels) and caspase 3/7 activity (right panels) analyses at 48 h post-transfection. N=3-5. FIG. 14C depicts RT-qPCR results of UtLM-1 or ht-UtLM cells that were transfected with siCon, siH19 or a TET3-specific siRNA (siTET3). RNA levels were measured by RT-qPCR 48 h later. n=3, One-way ANOVA with Dunnett post-test.

FIG. 15, comprising FIG. 15A through FIG. 15C, depicts the results that demonstrate that H19 regulates expression of HMGA2 and TET3 post-transcriptionally via let-7. n=3, One-way ANOVA with Tukey post-test. Data are representative of two independent experiments and are presented as mean±SEM. *p<0.05, **p<0.01. ns, not statistically significant. FIG. 15A depicts RT-qPCR and Western blot results of UtLM cells that were transfected with siCon+iCon (miRNA inhibitor control), siH19+iCon, or siH19+iLet7; RNA and proteins were isolated 48 h later and analyzed by RT-qPCR (left panels) or Western blot (right panels). FIG. 15B depicts schematics of human and mouse TET3 mRNAs, with numbers on top depicting positions of let-7-binding sites relative to the transcriptional start sites. Figures are not drawn to scale. FIG. 15C depicts RT-qPCR and Western blot results of UtLM cells that were transfected with siCon+iCon (miRNA inhibitor control), siH19+iCon, or siH19+iLet7; RNA and proteins were isolated 48 h later and analyzed by RT-qPCR (left panels) or Western blot (right panels).

FIG. 16, comprising FIG. 16A through FIG. 16D, depicts the results that demonstrate that TET3 affects DNA methylation and histone modifications of the MED12, TGFBR2 and TSP1 promoters. All data are representative of at least two independent experiments and are presented as mean±SEM. *p<0.05, **p<0.01. FIG. 16A depicts ChIP-qPCR results of UtLM cells that were transfected with siCon or siTET3 for 48 h, followed by ChIP-qPCR analysis. Data are presented as mean relative TET3 enrichment over input. n=3. Red numbers indicate nucleotide positions relative to the transcriptional start sites, with PCR products depicted as red-stripped bars. FIG. 16B depicts sequences of critical transcription regulatory regions (CTRR) of MED12, TGFBR2 and TSP1. The differentially methylated cytosine residues are marked in red. The red numbers mark the positions of the indicated nucleotides relative to the transcriptional start sites. FIG. 16C depicts QMSP results of UtLM cells that were transfected with siCon or siTET3 for 48 h, followed by QMSP analysis. n=3. FIG. 16D depicts ChIP-qPCR results UtLM cells that were transfected with siCon or siTET3 for 48 h, followed by ChIP-qPCR analysis. Data are presented as mean relative enrichment over input. n=3.

FIG. 17, comprising FIG. 17A through FIG. 17C, depicts the results that demonstrate that H19 and TET3 co-express with fibroid-promoting genes in vivo. FIG. 17A depicts the results of RT-qPCR analyses that were performed on RNAs extracted from human fibroids and matched myometrium tissues. Spearman correlation showed positive correlations between expression of H19 and TET3 (left panel) as well as TET3 and its target genes MED12, TGFBR2, and TSP1. No correlation between expression of H19 and HMGA2 at the RNA level was detected (right panel). Spearman correlation coefficient, p values, and sample numbers are presented. FIG. 17B depicts the results of Western blot analysis of HMGA2 in human fibroids and matched myometrium. N=3. Data are representative of two independent experiments and are presented as mean±SEM. FIG. 17C depicts the results of RT-qPCR analyses that were performed on RNAs extracted from human fibroids and matched myometrium tissues. Spearman correlation showed positive correlations between expression of H19 and TET3 (left panel) as well as TET3 and its target genes MED12, TGFBR2, and TSP1 in a statistically significant manner.

FIG. 18, comprising FIG. 18A through FIG. 18D, depicts the results that demonstrate that progesterone and estradiol upregulate fibroid-promoting genes in a H19-dependent manner. All data are representative of at least two independent experiments and are presented as mean±SEM. *p<0.05, **p<0.01. ns, not statistically significant. FIG. 18A depicts RT-qPCR results of ht-UtLM cells that were treated with Vehicle (Veh, as a negative control), estradiol €, progesterone (P), or E and P together for 24 h. RNAs were extracted and H19 levels were determined by RT-qPCR analysis. N=3, One-way ANOVA with Dunnett post-test. FIG. 18B depicts RT-qPCR results of ht-UtLM cells that were transfected with siCon or siH19 for 24 h, followed by addition of Veh (−) or E+P (+) for an additional 24 h. RNA levels of the indicated genes were determined by RT-qPCR analysis. N=3, One-way ANOVA with Tukey post-test. FIG. 18C depicts protein levels that were determined by Western blot analysis, with quantifications presented on the right. Each group was loaded in triplicate. N=3. In C, TSP1 protein was isolated at 72 h post-transfection. N=3, One-way ANOVA with Tukey post-test. FIG. 18D depicts a proposed model.

FIG. 19, comprising FIG. 19A through FIG. 19F, depicts inflammatory cytokines (TNF-α and IL-1β) and Hcy upregulate expression of H19 and TGF-β signaling genes in a H19-dependent manner in Ecs. All experiments were performed three times with one set of representative results shown. Error bars were calculated based on triplicate PCR reactions. *p<0.05; **p<0.01; ns, not statistically significant compared to control. FIG. 19A depicts the results of RT-qPCR analysis of H19 expression in control (vehicle) HUVECs. Gapdh and β-tubulin were used for normalization of template loading. FIG. 19B depicts the results of RT-qPCR analysis of H19 expression in control (vehicle) and cytokine-treated (overnight) HUVECs. Gapdh and β-tubulin were used for normalization of template loading. FIG. 19C depicts the results of RT-qPCR analysis of H19 expression in Hcy-treated (overnight) HUVECs. Gapdh and β-tubulin were used for normalization of template loading. FIG. 19D depicts the results of RT-qPCR analysis of expression of H19, TET1, TGFBR2, TSP1 and β-tubulin 48 h after transfection of HUVECs with control siRNA (siCon) or H19-specific RNA (siH19). FIG. 19E depicts the results of RT-qPCR analysis of expression of indicated genes 48 h after transfection of HUVECs with empty vector (Vec) or human H19-expressing plasmid (pH19). FIG. 19F depicts the results of RT-qPCR analysis of expression of H19 (top left), TET1 (top right), TGFBR2 (bottom left) and TSP1 (bottom right) following transfection of siCon (−) or siH19 (+) for 24 h and treatment with TNF-α (10 ng/ml) (+) or vehicle (−) for an additional 24 h.

FIG. 20, comprising FIG. 20A through FIG. 20C, depicts the results that demonstrate that H19 regulates TET1 expression posttranscriptionally via reducing the bioavailability of let-7. All transfection experiments were carried out three times with one set of representative results shown. Error bars were calculated based on triplicate PCR reactions or protein loading. *p<0.05; **p<0.01. FIG. 20A depicts schematics of human and mouse TET1 mRNAs, with numbers on top depicting positions of let-7-binding sites relative to the transcriptional start sites. Figures are not drawn to scale. FIG. 20B depicts RT-qPCR results of HUVECs that were transfected with a mixture of siCon and iCon, siH19 and iCon, or siH19 and iLet7. RNA and protein were isolated 48 h later and analyzed by RT-qPCR. FIG. 20C depicts Western blot results of HUVECs that were transfected with a mixture of siCon and iCon, siH19 and iCon, or siH19 and iLet7. RNA and protein were isolated 48 h later and analyzed by Western blot. Protein samples were loaded in triplicate, with quantification of TET1 protein shown on the bottom. GAPDH was used as a loading control.

FIG. 21, comprising FIG. 21A through FIG. 21G, depicts TET1 affects promoter methylation and histone modifications of TGFBR2 and TSP1. All transfection experiments were performed three times with one set of representative results shown. Error bars were calculated based on triplicate PCR reactions or protein loading. *p<0.05; **p<0.01; ns, not statistically significant compared to control. FIG. 21A depicts RT-qPCR results of HUVECs that were transfected with siCon or siTET1. RNA and protein were isolated 48 h later and analyzed by RT-qPCR. FIG. 21B depicts Western blot results of HUVECs that were transfected with siCon or siTET1. RNA and protein were isolated 48 h later and analyzed by Western blot. Protein samples were loaded in triplicate, with quantification of TGFBR2 and TSP1 proteins shown on the bottom. FIG. 21C depicts ChIP-qPCR results of HUVECs that were transfected with siCon or siTET1, followed by ChIP-qPCR analysis. Data are presented as mean relative TET1 enrichment over input. Blue numbers indicate nucleotide positions relative to the transcriptional start sites, with PCR products depicted as purple bars. FIG. 21D depicts sequences of critical transcription regulatory regions (CTRR) of TGFBR2 and TSP1. The differentially methylated cytosine residues are marked in red. The red numbers mark the positions of the indicated nucleotides relative to the transcriptional start sites. FIG. 21E depicts HUVECs that were transfected with siCon or siTET1 for 48 h, followed by QMSP analysis. FIG. 21F depicts ChIP-qPCR results of HUVECs (TGFBR2) that were transfected with siCon or siTET1 for 48 h, followed by ChIP-qPCR analysis. *p<0.05; **p<0.01; ns, not statistically significant compared to control. FIG. 21G depicts ChIP-qPCR results of HUVECs (TSP1) that were transfected with siCon or siTET1 for 48 h, followed by ChIP-qPCR analysis.

FIG. 22, comprising FIG. 22A through FIG. 22D, depicts that H19 regulates TGFBR2 and TSP1 expression in a TET1-dependent manner. *p<0.05; **p<0.01; ***p<0.0001; ns, not statistically significant compared to control. All transfection experiments were carried out three times with one set of representative results shown. Error bars were calculated based on triplicate PCR reactions or protein loading. FIG. 22A depicts RT-qPCR results of HUVECs that were transfected with siCon, siH19, or siH19 plus pTET1 (human TET1-expressing plasmid). RNA and protein were extracted 48 h and 72 h later, respectively, and analyzed by RT-qPCR. FIG. 22B depicts Western blot results of HUVECs that were transfected with siCon, siH19, or siH19 plus pTET1 (human TET1-expressing plasmid). RNA and protein were extracted 48 h and 72 h later, respectively, and analyzed by Western blot. FIG. 22C depicts HUVECs that were transfected with siCon, siH19, or siH19 plus pTET1 (human TET1-expressing plasmid). RNA and protein were extracted 48 h and 72 h later, respectively, and analyzed with quantification of TET1, TGFBR2, and TSP1 proteins. FIG. 22D depicts a proposed regulatory pathway.

FIG. 23, comprising FIG. 23A and FIG. 23B, depicts the results that demonstrate that overexpression of H19 or TET1 promotes TGF-β signaling and EndMT marker expression in vitro. FIG. 23A depicts Western blot results of HUVECs that were transfected with empty vector, pH19, or pTET1. Proteins were extracted 48 h post transfection and analyzed by Western blot. FIG. 23B depicts HUVECs that were transfected with empty vector, pH19, or pTET1. Proteins were extracted 48 h post transfection and analyzed with quantification of indicated proteins. *p<0.05;**p<0.01. Transfection experiments were carried out three times with one set of representative results shown. Error bars were calculated based on triplicate protein loading.

FIG. 24, comprising FIG. 24A through FIG. 24D, depicts that H19 depletion abrogates endothelial activation of TGF-β signaling and EndMT marker expression in vivo. FIG. 24A depicts the results of ELISA analysis of serum TNF-α levels of WT and KO mice 2 weeks after initial injection of STZ (+) or vehicle (−), n=4 animals per group. FIG. 24B depicts the results of RT-qPCR analysis of expression of H19, TET1, TGFBR2, and TSP1 in lung ECs collected from WT and KO mice 5 weeks following initial injection of STZ (+) or vehicle (−). n=3 animals per group. FIG. 24C depicts the results of Western blot analysis of indicated proteins in lung ECs collected from WT and KO mice 5 weeks following initial injection of STZ (+) or vehicle (−). n=3 animals per group. FIG. 24D depicts the results of quantification of proteins shown in FIG. 24C. *p<0.05; **p<0.01; ns, not statistically significant compared to control.

FIG. 25, comprising FIG. 25A through FIG. 25E, depicts H19 and TET1 expression in ECs in human coronary arteries. Left main coronary arteries from patients with no/mild, moderate, and severe CAD were assessed. n=5 in each group. FIG. 25A depicts representative images of RNA ISH staining of left main coronary arteries from patients for H19 (blue) and CD31 (red), with nuclei stained in purple. Scale bar: 100 μm. FIG. 25B depicts (top panel) percentage of H19+ ECs or TET1+ ECs in the lumen. **p<0.01; ***p<0.001 compared with no/mild disease; 1-way ANOVA with Newman-Keuls post-hoc test for multiple comparison correction; and (bottom panel) scatter plots of H19+ ECs or TET1+ ECs and the I/M ratio. The corresponding Spearman's correlation coefficient (r) between H19+ ECs or TET1+ ECs and the I/M ratio and the p value are shown. FIG. 25C depicts representative images of immunofluorescence staining for TET1 (green) and CD31 (red), with nuclei stained with DAPI in blue. Scale bar: 100 μm. FIG. 25D depicts (top panel) percentage of H19+ ECs or TET1+ ECs in the lumen. **p<0.01; ***p<0.001 compared with no/mild disease; 1-way ANOVA with Newman-Keuls post-hoc test for multiple comparison correction; and (bottom panel) scatter plots of H19+ ECs or TET1+ ECs and the I/M ratio. The corresponding Spearman's correlation coefficient (r) between H19+ ECs or TET1+ ECs and the I/M ratio and the p value are shown. FIG. 25E depicts scatter plot of H19+ ECs and TET1+ ECs. The corresponding Spearman's correlation coefficient (r) between H19+ ECs and TET1+ ECs and the p value are shown.

FIG. 26, comprising FIG. 26A through FIG. 26E, depicts mesenchymal marker expression in the endothelium of human coronary arteries. Left main coronary arteries from patients with no/mild, moderate, and severe CAD were assessed. n=5 in each group. FIG. 26A depicts representative images of immunofluorescence staining for CD31(red), FN1(green), and VIM (green) in the endothelium of coronary arteries. Nuclei were stained with DAPI (blue). Scale bar: 100 μm. FIG. 26B depicts (top panel) percentage of FN1+ ECs or VIM+ ECs in the lumen. **p<0.01; ***p<0.001 compared with no/mild disease; 1-way ANOVA with Newman-Keuls post-hoc test for multiple comparison correction; and (bottom panel) scatter plots of FN1+ ECs or VIM+ ECs and the I/M ratio. The corresponding Spearman's correlation coefficient (r) between FN1 or VIM and the I/M ratio and the p value are shown. FIG. 26C depicts representative images of immunofluorescence staining for CD31(red), FN1(green), and VIM (green) in the endothelium of coronary arteries. Nuclei were stained with DAPI (blue). Scale bar: 100 FIG. 26D depicts (top panel) percentage of FN1+ ECs or VIM+ ECs in the lumen. **p<0.01; ***p<0.001 compared with no/mild disease; 1-way ANOVA with Newman-Keuls post-hoc test for multiple comparison correction; and (bottom panel) scatter plots of FN1+ ECs or VIM+ ECs and the I/M ratio. The corresponding Spearman's correlation coefficient (r) between FN1 or VIM and the I/M ratio and the p value are shown. FIG. 26E depicts scatter plots of H19+ ECs and FN1+ ECs; H19+ ECs and VIM+ ECs; TET1+ ECs and FN1+ ECs; and TET1+ ECs and VIM+ ECs.

### DETAILED DESCRIPTION

The present invention is based in part on the discovery that the development and progression of various diseases and disorders, such as fibrosis, cancer, and diabetes, is associated with an increased ten-eleven translocation protein (TET) level or activity, an increased H19 level or activity, an increased transforming growth factor (TGF) level or activity, or any combination thereof in a subject. Thus, the invention relates to TET, H19, TGF, hepatocyte nuclear factor (HNF), or isoforms thereof, as novel pharmacological targets and/or biomarkers for the treatment of diseases or disorders. The present invention also provides methods relating to the pharmacological targets of the invention that can be used to establish and evaluate treatment regimens for the diseases or disorders of the invention.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent depending on the context in which it is used. As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “amplification” refers to the operation by which the number of copies of a target nucleotide sequence present in a sample is multiplied.

A disease or disorder is “alleviated” if the severity of a sign or symptom of the disease or disorder, the frequency with which such a sign or symptom is experienced by a patient, or both, is reduced.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A “fibrosis” or “fibrotic disease or disorder,” as used herein, relates to a disease or disorder involving fibrosis, which may be due to e.g. chronic inflammation or repair and reorganization of tissues. Fibrosis is the formation or development of excess fibrous connective tissue in an organ or tissue as a reparative or reactive process, as opposed to formation of fibrous tissue as a normal constituent of an organ or tissue, including the kidneys, heart, lungs, liver, skin and joints. Fibrotic diseases include, without limitation, pulmonary fibrosis (including idiopathic pulmonary fibrosis and cystic fibrosis), renal fibrosis, hepatic cirrhosis, endomyocardial fibrosis, atrial fibrosis, mediastinal fibrosis, myelofibrosis, retroperitoneal fibrosis, progressive massive fibrosis of the lungs, nephrogenic systemic fibrosis, Crohn's disease, keloid, Scleroderma/systemic sclerosis of the skin or the lungs, arthrofibrosis, Peyronie's disease, Dupuytren's contracture, some forms of adhesive capsulitis, peritoneal fibrosis, and skin fibrosis. In a particular embodiment, the fibrotic disease is selected from the group consisting of idiopathic pulmonary fibrosis, peritoneal fibrosis and skin fibrosis.

The term “pulmonary disease or disorder” encompasses lung (pulmonary) fibrosis and pulmonary diseases or disorders with a fibrotic component selected from idiopathic pulmonary fibrosis, other interstitial pneumonias (IP) such as giant cell interstitial pneumonia, non-specific IP, cryptogenic organizing pneumonia, collagen vascular disease-associated IP, and drug-induced IP, also sarcodosis, cystic fibrosis, respiratory distress syndrome, granulomatosis, silicosis, asbestosis, systemic scleroderma involving the lung, as well as fibrosis and remodeling in asthma or chronic obstructive pulmonary disease (COPD).

“Cancer,” as used herein, refers to the abnormal growth or division of cells. Generally, the growth and/or life span of a cancer cell exceeds, and is not coordinated with, that of the normal cells and tissues around it. Cancers may be benign, pre-malignant or malignant. Cancer occurs in a variety of cells and tissues, including, but not limited to, the oral cavity (e.g., mouth, tongue, pharynx, etc.), digestive system (e.g., esophagus, stomach, small intestine, colon, rectum, liver, bile duct, gall bladder, pancreas, etc.), respiratory system (e.g., larynx, lung, bronchus, etc.), bones, joints, skin (e.g., basal cell, squamous cell, meningioma, etc.), breast, genital system, (e.g., uterus, ovary, prostate, testis, etc.), urinary system (e.g., bladder, kidney, ureter, etc.), eye, nervous system (e.g., brain, etc.), endocrine system (e.g., thyroid, etc.), soft tissues (e.g., muscle, fat, etc.), and hematopoietic system (e.g., lymphoma, myeloma, leukemia, acute lymphocytic leukemia, chronic lymphocytic leukemia, acute myeloid leukemia, chronic myeloid leukemia, etc.).

The term “inhibit,” as used herein, means to suppress or block an activity or function by at least about ten percent relative to a control value. In various embodiments, the activity is suppressed or blocked by at least 50% compared to a comparator value, or by at least 55%, or by at least 60%, or by at least 65%, or by at least 70%, or by at least 75%, or by at least 80%, or by at least 85%, or by at least 90%, or by at least 95%.

As used herein, the term “diagnosis” refers to the determination of the presence of a disease or disorder. In various embodiments of the present invention, methods for making a diagnosis are provided which permit determination of the presence of a particular disease or disorder.

A “therapeutic” treatment is a treatment administered to a subject who exhibits signs or symptoms of a disease or disorder, for the purpose of diminishing or eliminating the severity and/or frequency of those signs or symptoms.

As used herein, “treating a disease or disorder” means reducing the severity and/or frequency with which at least one sign or symptom of the disease or disorder is experienced by a patient.

The term “solvate” in accordance with this invention should be understood as meaning any form of the active compound in accordance with the invention in which said compound is bonded by a non-covalent bond to another molecule (normally a polar solvent), including especially hydrates and alcoholates.

The terms “effective amount” and “pharmaceutically effective amount” refer to a sufficient amount of an agent to provide the desired biological result. That result can be reduction and/or alleviation of a sign, symptom, or cause of a disease or disorder, or any other desired alteration of a biological system. An appropriate effective amount in any individual case may be determined by one of ordinary skill in the art using routine experimentation.

A “therapeutically effective amount” refers to that amount which provides a therapeutic effect for a given disease or disorder and administration regimen. In particular, “therapeutically effective amount” means an amount that is effective to prevent, alleviate or ameliorate the severity and/or frequency of at least one sign or symptom of the disease or disorder, or prolong the survival of the subject being treated, which may be a human or non-human animal. Determination of a therapeutically effective amount is within the skill of the person skilled in the art.

As used herein, the term “pharmaceutical composition” refers to a mixture of at least one compound of the invention with other chemical components and entities, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

“Pharmaceutically acceptable” refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

The term “nutritional composition” may be a food product intended for human consumption, for example, a beverage, a drink, a bar, a snack, an ice cream, a dairy product, for example a chilled or a shelf-stable dairy product, a fermented dairy product, a drink, for example a milk-based drink, an infant formula, a growing-up milk, a confectionery product, a chocolate, a cereal product such as a breakfast cereal, a sauce, a soup, an instant drink, a frozen product intended for consumption after heating in a microwave or an oven, a ready-to-eat product, a fast food or a nutritional formula.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

“Instructional material,” as that term is used herein, includes a publication, a recording, a diagram, or any other medium of expression which can be used to communicate the usefulness of the nucleic acid, peptide, and/or compound of the invention in the kit for identifying, diagnosing or alleviating or treating the various diseases or disorders recited herein. Optionally, or alternately, the instructional material may describe one or more methods of identifying, diagnosing or alleviating the diseases or disorders in a cell or a tissue of a subject. The instructional material of the kit may, for example, be affixed to a container that contains one or more components of the invention or be shipped together with a container that contains the one or more components of the invention. Alternatively, the instructional material may be shipped separately from the container with the intention that the recipient uses the instructional material and the components cooperatively.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule also consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In some instances, the first region comprises a first portion and the second region comprises a second portion, whereby, when the first and second portions are arranged in an antiparallel fashion, at least about 50%, at least about 75%, at least about 90%, or at least about 95% of the nucleotide residues of the first portion are capable of base pairing with nucleotide residues in the second portion. In some instances, all nucleotide residues of the first portion are capable of base pairing with nucleotide residues in the second portion.

The term “DNA” as used herein is defined as deoxyribonucleic acid.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

The term “fusion polypeptide” refers to a chimeric protein containing a protein of interest (e.g., luciferase) joined to a heterologous sequence (e.g., a non-luciferase amino acid or protein).

The term “homology” refers to a degree of complementarity. There may be partial homology or complete homology (i.e., identity). Homology is often measured using sequence analysis software (e.g., Sequence Analysis Software Package of the Genetics Computer Group. University of Wisconsin Biotechnology Center. 1710 University Avenue. Madison, Wis. 53705). Such software matches similar sequences by assigning degrees of homology to various substitutions, deletions, insertions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “label” when used herein refers to a detectable compound or composition that is conjugated directly or indirectly to a probe to generate a “labeled” probe. The label may be detectable by itself (e.g. radioisotope labels or fluorescent labels) or, in the case of an enzymatic label, may catalyze chemical alteration of a substrate compound or composition that is detectable (e.g., avidin-biotin). In some instances, primers can be labeled to detect a PCR product.

Assays for amplification of the known sequence are also disclosed. For example primers for PCR may be designed to amplify regions of the sequence. For RNA, a first reverse transcriptase step may be used to generate double stranded DNA from the single stranded RNA. The array may be designed to detect sequences from an entire genome; or one or more regions of a genome, for example, selected regions of a genome such as those coding for a protein or RNA of interest; or a conserved region from multiple genomes; or multiple genomes, arrays and methods of genetic analysis using arrays is described in Cutler, et al., 2001, Genome Res. 11(11): 1913-1925 and Warrington, et al., 2002, Hum Mutat 19:402-409 and in US Patent Pub No 20030124539, each of which is incorporated herein by reference in its entirety.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “nucleic acid” refers to a polynucleotide and includes poly-ribonucleotides and poly-deoxyribonucleotides. Nucleic acids according to the present invention may include any polymer or oligomer of pyrimidine and purine bases, preferably cytosine, thymine, and uracil, and adenine and guanine, respectively. (See Albert L. Lehninger, Principles of Biochemistry, at 793-800 (Worth Pub. 1982) which is herein incorporated in its entirety for all purposes). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

As used herein, the term “polymerase chain reaction” (“PCR”) refers to the method of K. B. Mullis (U.S. Pat. Nos. 4,683,195 4,683,202, and 4,965,188, hereby incorporated by reference), which describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified”. As used herein, the terms “PCR product,” “PCR fragment,” “amplification product” or “amplicon” refer to the resultant mixture of compounds after two or more cycles of the PCR steps of denaturation, annealing and extension are complete. These terms encompass the case where there has been amplification of one or more segments of one or more target sequences. As used herein, the term “probe” refers to an oligonucleotide (i.e., a sequence of nucleotides), whether occurring naturally as in a purified restriction digest or produced synthetically, recombinantly or by PCR amplification, that is capable of hybridizing to another oligonucleotide of interest. A probe may be single-stranded or double-stranded. Probes are useful in the detection, identification and isolation of particular gene sequences.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or any combination thereof.

As used herein, “polynucleotide” includes cDNA, RNA, DNA/RNA hybrid, antisense RNA, ribozyme, genomic DNA, synthetic forms, and mixed polymers, both sense and antisense strands, and may be chemically or biochemically modified to contain non-natural or derivatized, synthetic, or semi-synthetic nucleotide bases. Also, contemplated are alterations of a wild type or synthetic gene, including but not limited to deletion, insertion, substitution of one or more nucleotides, or fusion to other polynucleotide sequences.

The term “primer” refers to an oligonucleotide capable of acting as a point of initiation of synthesis along a complementary strand when conditions are suitable for synthesis of a primer extension product. The synthesizing conditions include the presence of four different deoxyribonucleotide triphosphates and at least one polymerization-inducing agent such as reverse transcriptase or DNA polymerase. These are present in a suitable buffer, which may include constituents which are co-factors or which affect conditions such as pH and the like at various suitable temperatures. A primer is preferably a single strand sequence, such that amplification efficiency is optimized, but double stranded sequences can be utilized.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based in part on the discovery that the development and progression of various diseases or disorders is associated with an increased TET level, an increased H19 level, an increased level of TGF signaling, or any combination thereof in a subject. Thus, the invention relates to compositions and methods relating to biomarkers that can be used for treating or preventing diseases or disorders associated with an increased TET level, an increased H19 level, an increased level of TGF signaling, or any combination thereof, in a subject in need thereof. As described herein, an increased level of TET, an increased level of H19, an increased level of TGF signaling, or any combination thereof, is demonstrated to be a useful diagnostic and prognostic biomarker for fibrosis, cancer, and diabetes. The present invention also relates to TET, H19, TGF, HNF, or isoforms thereof, as novel pharmacological targets for the treatment of diseases or disorders. The present invention provides compositions and methods for the inhibition of TET, the inhibition of H19, the inhibition of TGF, or any combination thereof, for the treatment and management of diseases or disorders associated with increased TET level, increased H19 level, increased level of TGF signaling, or any combination thereof. The present invention also provides methods relating to the pharmacological targets of the invention that can be used as biomarkers to establish and evaluate treatment plans for the diseases or disorders of the invention.

### Compositions and Methods

In one aspect, the present invention relates to TET, H19, TGF, HNF, or isoforms thereof, as novel biomarkers and/or pharmacological targets for treatment or prevention of diseases or disorders in a subject. In one embodiment, the disease or disorder is a disease or disorder associated with increased TET level (e.g., TET expression, enzymatic activity of TET, interaction between TET and their interacting partners, RNA level or protein level or activity level, etc.). In one embodiment, the disease or disorder is a disease or disorder associated with increased H19 level (e.g., RNA level or protein level or activity level, etc.). In one embodiment, the disease or disorder is a disease or disorder associated with increased level of TGF signaling (e.g., RNA level or protein level or activity level, etc.). In various embodiments, the disease or disorder is a disease or disorder associated with increased TET level (e.g., TET expression, enzymatic activity of TET, interaction between TET and their interacting partners, RNA level or protein level or activity level, etc.), increased H19 level (e.g., RNA level or protein level or activity level, etc.), increased level of TGF signaling (e.g., RNA level or protein level or activity level, etc.), or any combination thereof in a subject. In one embodiment, the method comprises administering a treatment to a subject in need thereof.

In one embodiment, the invention is a method of treatment or prevention of a disease or disorder associated with increased TET level (e.g., TET expression, enzymatic activity of TET, interaction between TET and their interacting partners, RNA level or protein level or activity level, etc.), increased H19 level (e.g., RNA level or protein level or activity level, etc.), increased level of TGF signaling (e.g., RNA level or protein level or activity level, etc.), or any combination thereof in the subject. In one embodiment, the invention is a biomarker for diagnosing a disease or disorder associated with increased TET level, increased H19 level, increased level of TGF signaling, or any combination thereof in the subject. In one embodiment, the invention provides a biomarker for assessing prognosis of a disease or disorder associated with increased TET level, increased H19 level, increased TGF level, increased level of TGF signaling, or any combination thereof in a subject. In one embodiment, the invention provides a biomarker for assessing the risk of developing a disease or disorder associated with increased TET level, increased H19 level, increased TGF level, or any combination thereof in the subject in a subject. In one embodiment, the invention a method of diagnosing subject as being at risk of developing a disease or disorder, comprising the detection of an increased TET level, an increased H19 level, an increased TGF level, an increased level of TGF signaling, or any combination thereof, and preventing the subject from developing the disease or disorder by administering an inhibitor as described elsewhere herein. In one embodiment, the invention is a method of detecting a disease or disorder in a subject, comprising the detection of an increased TET level, an increased H19 level, an increased TGF level, an increased level of TGF signaling, or any combination thereof. In one embodiment, the invention is a method of diagnosing subject as having a disease or disorder, comprising the detection of an increased TET level, an increased H19 level, an increased TGF level, an increased level of TGF signaling, or any combination thereof. In one embodiment, the invention is a method of diagnosing subject as having a disease or disorder and treating the subject for the disease or disorder, comprising the detection of an increased TET level, an increased H19 level, an increased TGF level, an increased level of TGF signaling, or any combination thereof, and administering an inhibitor as described elsewhere herein.

In one embodiment, the TET is a ten-eleven translocation protein 1 (TET1). In one embodiment, the TET is a ten-eleven translocation protein 2 (TET2). In one embodiment, the TET is a ten-eleven translocation protein 3 (TET3). In various embodiments, the TET is a TET1, TET2, TET3, or any combination thereof.

In one embodiment, the TGF signaling comprises a transforming growth factor alpha (TGF-α). In one embodiment, the TGF signaling comprises a TGF-α isoform. In one embodiment, the TGF signaling comprises a transforming growth factor beta (TGF-β). In one embodiment, the TGF signaling comprises a TGF-β isoform. In one embodiment, the TGF signaling comprises a TGF-β receptor 1 (TGFBR1). In one embodiment, the TGF signaling comprises a TGF-β receptor 2 (TGFBR2). In one embodiment, the TGF signaling comprises a Smad protein 2 (Smad2). In one embodiment, the TGF signaling comprises a Smad protein 3 (Smad3). In one embodiment, the TGF signaling comprises a Smad protein 4 (Smad4). In one embodiment, the TGF signaling comprises a phosphorylated SMAD2 (p-SMAD2). In one embodiment, the TGF signaling comprises a phosphorylated SMAD 3 (p-SMAD3). In one embodiment, the TGF signaling comprises a phosphorylated SMAD 4 (p-SMAD4). In various embodiments, the TGF signaling comprises at least one component selected from the group consisting of TGF-α, TGF-α isoform, TGF-β, TGF-β isoform, TGFBR1, TGFBR2, Smad2, Smad3, Smad4, p-SMAD2, p-SMAD3, p-SMAD4, and any combination thereof.

In one embodiment, the TGF-β is a transforming growth factor beta 1 (TGF-β1). In one embodiment, the TGF-β isoform is a TGF-β1 isoform. In one embodiment, the TGF-β is a transforming growth factor beta 2 (TGF-β2). In one embodiment, the TGF-β isoform is a TGF-β2 isoform. In one embodiment, the TGF-β is a transforming growth factor beta 3 (TGF-β3). In one embodiment, the TGF-β isoform is a TGF-β3 isoform. In one embodiment, the TGF-β is a transforming growth factor beta 4 (TGF-β4). In one embodiment, the TGF-β is a TGF-β4 isoform. In various embodiments, the TGF-β is TGF-β1, TGF-β2, TGF-β3, TGF-β4, or any combination thereof. In various embodiments, the TGF-β isoform is TGF-β1 isoform, TGF-β2 isoform, TGF-β3 isoform, TGF-β4 isoform, or any combination thereof.

In one embodiment, the treatment comprises a reduction of TET level in a subject in need thereof. In one embodiment, the treatment comprises a reduction of TET activity in a subject in need thereof. In one embodiment, the treatment comprises a reduction of H19 level in a subject in need thereof. In one embodiment, the treatment comprises a reduction of H19 activity in a subject in need thereof. In one embodiment, the treatment comprises a reduction of TGF signaling in a subject in need thereof. In one embodiment, the treatment comprises a reduction of TGF activity in a subject in need thereof. In one embodiment, the treatment comprises a reduction of hepatocyte nuclear factor (HNF) level in a subject in need thereof. In one embodiment, the treatment comprises a reduction of HNF activity in a subject in need thereof. In one embodiment, the treatment comprises a reduction of HNF isoform level in a subject in need thereof. In one embodiment, the treatment comprises a reduction of HNF isoform activity in a subject in need thereof. In various embodiments, the treatment comprises a reduction of TET level or activity, a reduction of H19 level or activity, a reduction of TGF signaling or activity, a reduction of HNF level or activity, a reduction of HNF isoform level or activity, or any combination thereof, in a subject in need thereof.

In one embodiment, the HNF is a hepatocyte nuclear factor 1 alpha (HNF1α). In one embodiment, the HNF is a hepatocyte nuclear factor 1 beta (HNF1β). In one embodiment, the HNF is a hepatocyte nuclear factor 3 alpha (HNF3α). In one embodiment, the HNF is a hepatocyte nuclear factor 3 beta (HNF3β). In one embodiment, the HNF is a hepatocyte nuclear factor 4 alpha (HNF4α). In one embodiment, the HNF is a hepatocyte nuclear factor 4 gamma (HNF4γ). In one embodiment, the HNF is a hepatocyte nuclear factor 6 alpha (HNF6α). In one embodiment, the HNF is a hepatocyte nuclear factor 6 beta (HNF6β). In various embodiments, the HNF is HNF1α, HNF1β, HNF3α, HNF3β, HNF4α, HNF4γ, HNF6α, HNF6β, or any combination thereof.

In one embodiment, the HNF isoform is a P1-derived hepatocyte nuclear factor 4 alpha (HNF4α P1) isoform. In one embodiment, the HNF4α P1 isoform is a P1-derived HNF4α1 isoform. In one embodiment, the HNF4α P1 isoform is a P1-derived HNF4α2 isoform. In one embodiment, the HNF4α P1 isoform is a P1-derived HNF4α3 isoform. In one embodiment, the HNF4α P1 isoform is a P1-derived HNF4α4 isoform. In one embodiment, the HNF4α P1 isoform is a P1-derived HNF4α5 isoform. In one embodiment, the HNF4α P1 isoform is a P1-derived HNF4α6 isoform. In various embodiments, the HNF4α P1 isoform is a P1-derived HNF4α1 isoform, P1-derived HNF4α2 isoform, P1-derived HNF4α3 isoform, P1-derived HNF4α4 isoform, P1-derived HNF4α5 isoform, P1-derived HNF4α6 isoform, or any combination thereof.

In one embodiment, the HNF isoform is a P2-derived hepatocyte nuclear factor 4 alpha (HNF4α P2) isoform. In one embodiment, the HNF4α P2 isoform is a P2-derived HNF4α7 isoform. In one embodiment, the HNF4α P2 isoform is a P2-derived HNF4α8 isoform. In one embodiment, the HNF4α P2 isoform is a P2-derived HNF4α9 isoform. In various embodiments, the HNF4α P2 isoform is a P2-derived HNF4α7 isoform, P2-derived HNF4α8 isoform, P2-derived HNF4α9 isoform, or any combination thereof. In various embodiments, the HNF isoform is a combination of HNF4α P1 isoform and HNF4α P2 isoform.

In various embodiments, the method comprises a decrease of TET level or activity, decreased of H19 level or activity, decrease of TGF signaling, decrease of HNF level or activity, decrease of PEPCK level or activity, decrease of estradiol level or activity, decrease of progesterone level or activity, decrease of glucagon level or activity, decrease of MED12 level or activity, decrease of GRAF1 level or activity, decrease of SPARC level or activity, decrease of VIM level or activity, decrease of COL3A1 level or activity, decrease of COL4A1 level or activity, decrease of COL5A2 level or activity, decrease of HMGA2 level or activity, decrease of SLUG level or activity, decrease of p-SMAD2 level or activity, decrease of p-SMAD3 level or activity, decrease of p-SMAD4 level or activity, decrease of SM22-α level or activity, decrease of NOTCH3 level or activity, decrease of collagen 1 level or activity, decrease of fibronectin level or activity, decrease of G6PC level or activity, decrease of PGC level or activity, decrease of PGC-1a level or activity, decrease of TSP level or activity, decrease of TSP1 level or activity, decrease of FN1 level or activity, decrease of COL1A1 level or activity, decrease of T1MP1 level or activity, decrease of α-SMA level or activity, decrease of SMAD2 level or activity, decrease of SMAD3 level or activity, decrease of SMAD4 level or activity, decrease of LIN28B level or activity, or any combination thereof in a subject in need thereof. In various aspects of the invention, the method of treatment comprises the inhibition of TET, inhibition of H19, inhibition of TGF, inhibition of HNF, inhibition of PECK, inhibition of estradiol, inhibition of progesterone, inhibition of glucagon, inhibition of MED12, inhibition of GRAF1, inhibition of SPARC, inhibition of VIM, inhibition of COL3A1, inhibition of COL4A1, inhibition of COL5A2, inhibition of HMGA2, inhibition of SLUG, inhibition of p-SMAD2, inhibition of p-SMAD3, inhibition of p-SMAD4, inhibition of SM22-α, inhibition of NOTCH3, inhibition of collagen 1, inhibition of fibronectin, inhibition of G6PC, inhibition of PGC, inhibition of PGC-1α, inhibition of TSP, inhibition of TSP1, inhibition of FN1, inhibition of COL1A1, inhibition of T1MP1, inhibition of α-SMA, inhibition of SMAD2, inhibition of SMAD3, inhibition of SMAD4, inhibition of LIN28B, or any combination thereof. In various embodiments, the method comprises administering a therapeutically effective amount of a TET inhibitor, a H19 inhibitor, a TGF inhibitor, a HNF inhibitor, a PEPCK inhibitor, an estradiol inhibitor, a progesterone inhibitor, a glucagon inhibitor, a MED12 inhibitor, a GRAF1 inhibitor, a SPARC inhibitor, a VIM inhibitor, a COL3A1 inhibitor, a COL4A1 inhibitor, a COL5A2 inhibitor, an HMGA2 inhibitor, a SLUG inhibitor, a p-SMAD2 inhibitor, a p-SMAD3 inhibitor, a p-SMAD4 inhibitor, an SM22-α inhibitor, a NOTCH3 inhibitor, a collagen 1 inhibitor, a fibronectin inhibitor, a G6PC inhibitor, a PGC inhibitor, a PGC-1a inhibitor, a TSP inhibitor, a TSP1 inhibitor, a FN1 inhibitor, a COL1A1 inhibitor, a T1MP1 inhibitor, a α-SMA inhibitor, a SMAD2 inhibitor, a SMAD3 inhibitor, a SMAD4 inhibitor, a LIN28B inhibitor, or any combination thereof to a subject in need thereof.

In one embodiment, the method of treatment comprises administering a therapeutically effective amount of a TET inhibitor. In one embodiment, the TET inhibitor is a TET1 inhibitor. In one embodiment, the TET inhibitor is a TET2 inhibitor. In one embodiment, the TET inhibitor is a TET3 inhibitor. In various embodiments, the TET inhibitor is a TET1 inhibitor, a TET2 inhibitor, a TET3 inhibitor, or any combination thereof.

In one embodiment, the TET inhibitor is a nucleic acid. In one embodiment, the TET inhibitor is a peptide. In one embodiment, the TET inhibitor is a small molecule chemical compound. In one embodiment, the TET inhibitor is an siRNA. In one embodiment, the TET inhibitor is a ribozyme. In one embodiment, the TET inhibitor is an antisense nucleic acid. In one embodiment, the TET inhibitor is an aptamer. In one embodiment, the TET inhibitor is a peptidomimetic. In one embodiment, the TET inhibitor is an antibody. In one embodiment, the TET inhibitor is an antibody fragment. In one embodiment, the TET inhibitor is an induced protein degradation. In various embodiments, the TET inhibitor is a nucleic acid, a peptide, a small molecule chemical compound, an siRNA, a ribozyme, an antisense nucleic acid, an aptamer, a peptidomimetic, an antibody, an antibody fragment, an induced protein degradation, or any combination thereof.

In one aspect of the invention, the reduction of TET level comprises an inhibition of TET. In another aspect of the invention, the reduction of TET activity comprises an inhibition of TET. In one embodiment, the reduction of TET level comprises a reduction of H19 level or activity. In one embodiment, the reduction of TET activity comprises a reduction of H19 level or activity. In one embodiment, the reduction of TET level comprises a reduction of at least one TET co-factor. In one embodiment, the reduction of TET activity comprises a reduction of at least one TET co-factor. In one embodiment, the TET co-factor is α-ketoglutarate. In one embodiment, the TET co-factor is vitamin C. In one embodiment, the TET co-factor is iron. In one embodiment, the TET co-factor is 2-oxoglutarate. In various embodiments, the TET co-factor is α-ketoglutarate, vitamin C, iron, 2-oxoglutarate, or any combination thereof.

In one aspect of the invention, the reduction of TET level comprises a siRNA knockdown of TET. In another aspect of the invention, the reduction of TET activity comprises a siRNA knockdown of TET. In one embodiment, the siRNA knockdown of TET is a viral-mediated siRNA knockdown of TET. In one embodiment, the viral-mediated siRNA knockdown of TET comprises at least one AAV vector. In one embodiment, the siRNA knockdown of TET reduces TGF signaling.

In one aspect of the invention, the reduction of TET level reduces TGF signaling. In another aspect of the invention, the reduction of TET activity reduces TGF signaling. In one embodiment, the reduction of TET level blocks TGF signaling. In one embodiment, the reduction of TET activity blocks TGF signaling.

In one embodiment, the H19 inhibitor is a nucleic acid. In one embodiment, the H19 inhibitor is a peptide. In one embodiment, the H19 inhibitor is a small molecule chemical compound. In one embodiment, the H19 inhibitor is an siRNA. In one embodiment, the H19 inhibitor is a ribozyme. In one embodiment, the H19 inhibitor is an antisense nucleic acid. In one embodiment, the H19 inhibitor is an aptamer. In one embodiment, the H19 inhibitor is a peptidomimetic. In one embodiment, the H19 inhibitor is an antibody. In one embodiment, the H19 inhibitor is an antibody fragment. In one embodiment, the H19 inhibitor is an induced protein degradation. In various embodiments, the H19 inhibitor is a nucleic acid, a peptide, a small molecule chemical compound, an siRNA, a ribozyme, an antisense nucleic acid, an aptamer, a peptidomimetic, an antibody, an antibody fragment, an induced protein degradation, or any combination thereof.

In one aspect of the invention, the reduction of H19 level reduces TET level or activity. In another aspect of the invention, the reduction of H19 activity reduces TET level or activity. In one embodiment, the reduction of H19 level reduces let-7 level or activity. In one embodiment, the reduction of H19 activity reduces let-7 level or activity. In one embodiment, the reduction of H19 level blocks let-7. In one embodiment, the reduction of H19 activity blocks let-7.

In one embodiment, the method of treatment comprises administering a therapeutically effective amount of a TGF signaling inhibitor. In one embodiment, the TGF inhibitor is a TGFα inhibitor. In one embodiment, the TGF inhibitor is a TGFβ1 inhibitor. In one embodiment, the TGF inhibitor is a TGFβ2 inhibitor. In one embodiment, the TGF inhibitor is a TGFβ3 inhibitor. In one embodiment, the TGF inhibitor is a TGFβ4 inhibitor. In one embodiment, the TGF inhibitor is a TGFBR1 inhibitor. In one embodiment, the TGF inhibitor is a TGFBR2 inhibitor. In one embodiment, the TGF inhibitor is a Smad2 inhibitor. In one embodiment, the TGF inhibitor is a Smad3 inhibitor. In one embodiment, the TGF inhibitor is a Smad4 inhibitor. In various embodiments, the TGF inhibitor is a TGFα inhibitor, a TGFβ1 inhibitor, a TGFβ2 inhibitor, a TGFβ3 inhibitor, a TGFβ4 inhibitor, a TGFBR1 inhibitor, a TGFBR2 inhibitor, a Smad2 inhibitor, a Smad3 inhibitor, a Smad4 inhibitor, or any combination thereof.

In one embodiment, the TGF inhibitor is a nucleic acid. In one embodiment, the TGF inhibitor is a peptide. In one embodiment, the TGF inhibitor is a small molecule chemical compound. In one embodiment, the TGF inhibitor is an siRNA. In one embodiment, the TGF inhibitor is a ribozyme. In one embodiment, the TGF inhibitor is an antisense nucleic acid. In one embodiment, the TGF inhibitor is an aptamer. In one embodiment, the TGF inhibitor is a peptidomimetic. In one embodiment, the TGF inhibitor is an antibody. In one embodiment, the TGF inhibitor is an antibody fragment. In one embodiment, the TGF inhibitor is an induced protein degradation. In various embodiments, the TGF inhibitor is a nucleic acid, a peptide, a small molecule chemical compound, an siRNA, a ribozyme, an antisense nucleic acid, an aptamer, a peptidomimetic, an antibody, an antibody fragment, an induced protein degradation, or any combination thereof.

In one embodiment, the method of treatment comprises administering a therapeutically effective amount of a HNF inhibitor. In one embodiment, the HNF inhibitor is a HNF1α inhibitor. In one embodiment, the HNF inhibitor is a HNF1β inhibitor. In one embodiment, the HNF inhibitor is a HNF3α inhibitor. In one embodiment, the HNF inhibitor is a HNF3β inhibitor. In one embodiment, the HNF inhibitor is a HNF4α inhibitor. In one embodiment, the HNF inhibitor is a HNF4γ inhibitor. In one embodiment, the HNF inhibitor is a HN6a inhibitor. In one embodiment, the HNF inhibitor is a HNF6β inhibitor. In various embodiments, the HNF inhibitor is a HNF1α inhibitor, a HNF1β inhibitor, a HNF3α inhibitor, a HNF3β inhibitor, a HNF4α inhibitor, a HNF4γ inhibitor, a HN6a inhibitor, a HNF6β inhibitor, or any combination thereof.

In one embodiment, the HNF inhibitor is a nucleic acid. In one embodiment, the HNF inhibitor is a peptide. In one embodiment, the HNF inhibitor is a small molecule chemical compound. In one embodiment, the HNF inhibitor is an siRNA. In one embodiment, the HNF inhibitor is a ribozyme. In one embodiment, the HNF inhibitor is an antisense nucleic acid. In one embodiment, the HNF inhibitor is an aptamer. In one embodiment, the HNF inhibitor is a peptidomimetic. In one embodiment, the HNF inhibitor is an antibody. In one embodiment, the HNF inhibitor is an antibody fragment. In one embodiment, the HNF inhibitor is an induced protein degradation. In various embodiments, the HNF inhibitor is a nucleic acid, a peptide, a small molecule chemical compound, an siRNA, a ribozyme, an antisense nucleic acid, an aptamer, a peptidomimetic, an antibody, an antibody fragment, an induced protein degradation, or any combination thereof.

In one embodiment, the method of treatment comprises administering a therapeutically effective amount of a FOXA2 inhibitor. In one embodiment, the FOXA2 inhibitor is a nucleic acid. In one embodiment, the FOXA2 inhibitor is a peptide. In one embodiment, the FOXA2 inhibitor is a small molecule chemical compound. In one embodiment, the FOXA2 inhibitor is an siRNA. In one embodiment, the FOXA2 inhibitor is a ribozyme. In one embodiment, the FOXA2 inhibitor is an antisense nucleic acid. In one embodiment, the FOXA2 inhibitor is an aptamer. In one embodiment, the FOXA2 inhibitor is a peptidomimetic. In one embodiment, the FOXA2 inhibitor is an antibody. In one embodiment, the FOXA2 inhibitor is an antibody fragment. In one embodiment, the FOXA2 inhibitor is an induced protein degradation. In various embodiments, the FOXA2 inhibitor is a nucleic acid, a peptide, a small molecule chemical compound, an siRNA, a ribozyme, an antisense nucleic acid, an aptamer, a peptidomimetic, an antibody, an antibody fragment, an induced protein degradation, or any combination thereof.

In one embodiment, the method of treatment comprises administering a therapeutically effective amount of at least one let-7 promoter. In one embodiment, the let-7 promoter is a nucleic acid. In one embodiment, the let-7 promoter is a peptide. In one embodiment, the let-7 promoter is a small molecule chemical compound. In one embodiment, the let-7 promoter is an siRNA. In one embodiment, the let-7 promoter is a ribozyme. In one embodiment, the let-7 promoter is an antisense nucleic acid. In one embodiment, the let-7 promoter is an aptamer. In one embodiment, the let-7 promoter is a peptidomimetic. In one embodiment, the let-7 promoter is an antibody. In one embodiment, the let-7 promoter is an antibody fragment. In various embodiments, the let-7 promoter is a nucleic acid, a peptide, a small molecule chemical compound, an siRNA, a ribozyme, an antisense nucleic acid, an aptamer, a peptidomimetic, an antibody, an antibody fragment, or any combination thereof. In various aspects of the invention, the treatment further comprises a reduction of at least one selected from the group consisting of estradiol, progesterone, glucagon, MED12, GRAF1, SPARC, PEPCK, G6PC, PGC, PGC-1α, TSP, TSP1, FN1, VIM, COL1A1, COL3A1, COL4A1, COL5A2, HMGA2, SLUG, T1MP1, α-SMA, SMAD2, SMAD3, SMAD4, p-SMAD2, p-SMAD3, p-SMAD4, SM22-α, LIN28B, NOTCH3, collagen 1, fibronectin, and any combination thereof, in a subject in need thereof. In one embodiment, the treatment further comprises a reduction of estradiol in a subject in need thereof. In one embodiment, the treatment further comprises a reduction of progesterone in a subject in need thereof. In one embodiment, the treatment further comprises a reduction of glucagon in a subject in need thereof. In one embodiment, the treatment further comprises a reduction of MED12 in a subject in need thereof. In one embodiment, the treatment further comprises a reduction of GRAF1 in a subject in need thereof. In one embodiment, the treatment further comprises a reduction of SPARC in a subject in need thereof. In one embodiment, the treatment further comprises a reduction of PEPCK in a subject in need thereof. In one embodiment, the treatment further comprises a reduction of G6PC in a subject in need thereof. In one embodiment, the treatment further comprises a reduction of PGC in a subject in need thereof. In one embodiment, the treatment further comprises a reduction of PGC-1a in a subject in need thereof. In one embodiment, the treatment further comprises a reduction of TSP in a subject in need thereof. In one embodiment, the treatment further comprises a reduction of TSP1 in a subject in need thereof. In one embodiment, the treatment further comprises a reduction of FN1 in a subject in need thereof. In one embodiment, the treatment further comprises a reduction of VIM in a subject in need thereof. In one embodiment, the treatment further comprises a reduction of COL1A1 in a subject in need thereof. In one embodiment, the treatment further comprises a reduction of COL3A1 in a subject in need thereof. In one embodiment, the treatment further comprises a reduction of COL4A1 in a subject in need thereof. In one embodiment, the treatment further comprises a reduction of COL5A2 in a subject in need thereof. In one embodiment, the treatment further comprises a reduction of HMGA2 in a subject in need thereof. In one embodiment, the treatment further comprises a reduction of SLUG in a subject in need thereof. In one embodiment, the treatment further comprises a reduction of TIMP1 in a subject in need thereof. In one embodiment, the treatment further comprises a reduction of α-SMA in a subject in need thereof. In one embodiment, the treatment further comprises a reduction of SMAD2 in a subject in need thereof. In one embodiment, the treatment further comprises a reduction of SMAD3 in a subject in need thereof. In one embodiment, the treatment further comprises a reduction of SMAD4 in a subject in need thereof. In one embodiment, the treatment further comprises a reduction of p-SMAD2 in a subject in need thereof. In one embodiment, the treatment further comprises a reduction of p-SMAD3 in a subject in need thereof. In one embodiment, the treatment further comprises a reduction of p-SMAD4 in a subject in need thereof. In one embodiment, the treatment further comprises a reduction of LIN28B in a subject in need thereof. In one embodiment, the treatment further comprises a reduction of NOTCH3 in a subject in need thereof. In one embodiment, the treatment further comprises a reduction of collagen 1 in a subject in need thereof. In one embodiment, the treatment further comprises a reduction of fibronectin in a subject in need thereof.

In various aspects of the invention, the treatment further comprises reducing an expression of at least one selected from the group consisting of Tet3, Hnf4α, Pck1, G6pc, and any combination thereof, in a subject in need thereof. In one embodiment, the treatment further comprises reducing an expression of Tet3. In one embodiment, the treatment further comprises reducing an expression of Hnf4α. In one embodiment, the treatment further comprises reducing an expression of Pck1. In one embodiment, the treatment further comprises reducing an expression of G6pc.

In one embodiment, the disease or disorder associated with increased TET level, increased H19 level, increased level of TGF signaling, or any combination thereof is a disease or disorder associated with gluconeogenesis regulation. In one embodiment, the disease or disorder associated with increased TET level, increased H19 level, increased level of TGF signaling, or any combination thereof is a cardiovascular disease. In one embodiment, the cardiovascular disease is a cardiovascular disease involving endothelial dysfunction. In one embodiment, the disease or disorder associated with increased TET level, increased H19 level, increased level of TGF signaling, or any combination thereof is a coronary artery disease. In some embodiments, the disease or disorder associated with increased TET level, increased H19 level, increased level of TGF signaling, or any combination thereof is a fibrosis. The following are non-limiting examples of fibrosis that can be treated by the disclosed methods and compositions: a liver fibrosis, hepatic cirrhosis, chronic liver disease, viral hepatitis, alcoholic fatty liver disease, obesity, nonalcoholic fatty liver disease (NAFLD), type 1 diabetes, type 2 diabetes, pulmonary fibrosis, renal fibrosis, cardiac fibrosis, dermal fibrosis, cystic fibrosis, uterine fibrosis, leiomyomas, and pancreatic fibrosis. In some embodiments, the disease or disorder associated with increased TET level, increased H19 level, increased level of TGF signaling, or any combination thereof is a cancer. The following are non-limiting examples of cancer that can be treated by the disclosed methods and compositions: acute lymphoblastic; acute myeloid leukemia; adrenocortical carcinoma; adrenocortical carcinoma, childhood; appendix cancer; basal cell carcinoma; bile duct cancer, extrahepatic; bladder cancer; bone cancer; osteosarcoma and malignant fibrous histiocytoma; liposarcoma and anaplastic liposarcoma; brain stem glioma, childhood; brain tumor, adult; brain tumor, brain stem glioma, childhood; brain tumor, central nervous system atypical teratoid/rhabdoid tumor, childhood; central nervous system embryonal tumors; cerebellar astrocytoma; cerebral astrocytotna/malignant glioma; craniopharyngioma; ependymoblastoma; ependymoma; medulloblastoma; medulloepithelioma; pineal parenchymal tumors of intermediate differentiation; supratentorial primitive neuroectodermal tumors and pineoblastoma; visual pathway and hypothalamic glioma; brain and spinal cord tumors; breast cancer; bronchial tumors; Burkitt lymphoma; carcinoid tumor; carcinoid tumor, gastrointestinal; central nervous system atypical teratoid/rhabdoid tumor; central nervous system embryonal tumors; central nervous system lymphoma; cerebellar astrocytoma cerebral astrocytoma/malignant glioma, childhood; cervical cancer; chordoma, childhood; chronic lymphocytic leukemia; chronic myelogenous leukemia; chronic myeloproliferative disorders; colon cancer; colorectal cancer; craniopharyngioma; cutaneous T-cell lymphoma; esophageal cancer; Ewing family of tumors; extragonadal germ cell tumor; extrahepatic bile duct cancer; eye cancer, intraocular melanoma; eye cancer, retinoblastoma; biliary track cancer, cholangiocarcinoma, anal cancer, neuroendocrine tumors, small bowel cancer, gallbladder cancer; gastric (stomach) cancer; gastrointestinal carcinoid tumor; gastrointestinal stromal tumor (gist); germ cell tumor, extracranial; germ cell tumor, extragonadal; germ cell tumor, ovarian; gestational trophoblastic tumor; glioma; glioma, childhood brain stem; glioma, childhood cerebral astrocytoma; glioma, childhood visual pathway and hypothalamic; hairy cell leukemia; head and neck cancer; hepatocellular (liver) cancer; histiocytosis, langerhans cell; Hodgkin lymphoma; hypopharyngeal cancer; hypothalamic and visual pathway glioma; intraocular melanoma; islet cell tumors; kidney (renal cell) cancer; Langerhans cell histiocytosis; laryngeal cancer; leukemia, acute lymphoblastic; leukemia, acute myeloid; leukemia, chronic lymphocytic; leukemia, chronic myelogenous; leukemia, hairy cell; lip and oral cavity cancer; liver cancer; lung cancer, non-small cell; lung cancer, small cell; lymphoma, aids-related; lymphoma, burkitt; lymphoma, cutaneous T-cell; lymphoma, non-Hodgkin lymphoma; lymphoma, primary central nervous system; macroglobulinemia, Waldenstrom; malignant fibrous histiocvtoma of bone and osteosarcoma; medulloblastoma; melanoma; melanoma, intraocular (eye); Merkel cell carcinoma; mesothelioma; metastatic squamous neck cancer with occult primary; mouth cancer; multiple endocrine neoplasia syndrome, (childhood); multiple myeloma/plasma cell neoplasm; mycosis; fungoides; myelodysplastic syndromes; myelodysplastic/myeloproliferative diseases; myelogenous leukemia, chronic; myeloid leukemia, adult acute; myeloid leukemia, childhood acute; myeloma, multiple; myeloproliferative disorders, chronic; nasal cavity and paranasal sinus cancer; nasopharyngeal cancer; neuroblastoma; non-small cell lung cancer; oral cancer; oral cavity cancer; oropharyngeal cancer; osteosarcoma and malignant fibrous histiocytoma of bone; ovarian cancer; ovarian epithelial cancer; ovarian germ cell tumor; ovarian low malignant potential tumor; pancreatic cancer, islet cell tumors; papillomatosis; parathyroid cancer; penile cancer; pharyngeal cancer; pheochromocytoma; pineal parenchymal tumors of intermediate differentiation; pineoblastoma and supratentorial primitive neuroectodermal tumors; pituitary tumor; plasma celt neoplasm/multiple myeloma; pleuropulmonary blastoma; primary central nervous system lymphoma; prostate cancer; rectal cancer; renal cell (kidney) cancer; renal pelvis and ureter, transitional cell cancer; respiratory tract carcinoma involving the nut gene on chromosome 15; retinoblastoma; rhabdomyosarcoma; salivary gland cancer; sarcoma, ewing family of tumors; sarcoma, Kaposi; sarcoma, soft tissue; sarcoma, uterine; sezary syndrome; skin cancer (nonmelanoma); skin cancer (melanoma); skin carcinoma, Merkel cell; small cell lung cancer; small intestine cancer; soft tissue sarcoma; squamous cell carcinoma, squamous neck cancer with occult primary, metastatic; stomach (gastric) cancer; supratentorial primitive neuroectodermal tumors; T-cell lymphoma, cutaneous; testicular cancer; throat cancer; thymoma and thymic carcinoma; thyroid cancer; transitional cell cancer of the renal pelvis and ureter; trophoblastic tumor, gestational; urethral cancer; uterine cancer, endometrial; uterine sarcoma; vaginal cancer; vulvar cancer; Waldenstrom macroglobulinemia; Wilms tumor, and any combination thereof.

In one embodiment, the treatment comprises an intravenous administration. In one embodiment, the treatment comprises an oral administration. In one embodiment, the treatment comprises an aerosol administration. In one embodiment, the treatment comprises a parenteral administration. In one embodiment, the treatment comprises an ophthalmic administration. In one embodiment, the treatment comprises a pulmonary administration. In one embodiment, the treatment comprises a topical administration. In various embodiments, the treatment comprises at least one selected from the group consisting of an intravenous, oral, aerosol, parenteral, ophthalmic, pulmonary, and topical administration.

In various embodiments, the method of treatment includes, but is not limited to pharmacotherapy, surgery, radiation, and chemotherapy. In one embodiment, the method further comprises administering adjuvant radiotherapy to the subject in need thereof.

The present invention also provides methods relating to the biomarkers and/or the pharmacological targets of the invention that can be used to establish and evaluate treatment plans for the diseases or disorders of the invention. In one aspect, the present invention further provides methods relating to the biomarkers of the invention that can be used to establish and evaluate treatment plans for a subject at risk of developing disease or disorder associated with increased TET level (e.g., TET expression, enzymatic activity of TET, interaction between TET and their interacting partners, RNA level or protein level or activity level, etc.), increased H19 level (e.g., RNA level or protein level or activity level, etc.), increased level of TGF signaling (e.g., RNA level or protein level or activity level, etc.), or any combination thereof. In another aspect, the present invention also provides methods relating to the biomarkers of the invention that can be used to establish and evaluate treatment plans for a subject not at risk of developing a disease or disorder associated with increased TET level (e.g., TET expression, enzymatic activity of TET, interaction between TET and their interacting partners, RNA level or protein level or activity level, etc.), increased H19 level (e.g., RNA level or protein level or activity level, etc.), increased level of TGF signaling (e.g., RNA level or protein level or activity level, etc.), or any combination thereof. In one aspect, the present invention also provides methods for identifying agents for treating a disease or disorder associated with increased TET level, increased H19 level, increased level of TGF signaling, or any combination thereof that are appropriate or otherwise customized for a specific subject.

In various embodiments, the method of treatment comprises the detection of a differential expression of one or more biomarkers that indicate a treatment of the subject is needed. In one embodiment, the method of treatment comprises effecting a therapy based on increased biomarker level or activity. In one embodiment, a test sample from a subject, exposed to a therapeutic agent or a drug, can be taken and the level of one or more biomarkers can be determined. In various embodiments, the level of one or more biomarkers can be compared to a sample derived from the subject before and after treatment, or can be compared to samples derived from one or more subjects who have shown improvements in risk factors as a result of such treatment or exposure.

In one aspect, the biomarkers are used to monitor subjects undergoing treatments and therapies for a disease or disorder associated with increased TET level, increased H19 level, increased level of TGF signaling, or any combination thereof, subjects who have had a disease or disorder associated with increased TET level, increased H19 level, increased level of TGF signaling, or any combination thereof, and subjects who are in remission of a previously diagnosed and treated disease or disorder associated with increased TET level, increased H19 level, increased level of TGF signaling, or any combination thereof. In one embodiment, the biomarkers are used to select or modify treatments in subjects having a disease or disorder associated with increased TET level, increased H19 level, increased level of TGF signaling, or any combination thereof, subjects who have had a disease or disorder associated with increased TET level, increased H19 level, increased level of TGF signaling, and subjects who are in remission of a previously diagnosed and treated disease or disorder associated with increased TET level, increased H19 level, increased level of TGF signaling, or any combination thereof.

In various embodiments, the biomarker is, MED12, GRAF1, SPARC, TET, TGF, HNF, PEPCK, G6PC, H19, PGC, PGC-1α, TSP, TSP1, FN1, VIM, COL1A1, COL3A1, COL4A1, COL5A2, HMGA2, SLUG, T1MP1, α-SMA, SMAD2, SMAD3, SMAD4, p-SMAD2, p-SMAD3, p-SMAD4, SM22-α, LIN28B, NOTCH3, collagen 1, fibronectin, Tet3, Hnf4α, Pck1, G6p, or any combination thereof. In various embodiments, the method of treatment comprises effecting a therapy based on increased MED12 level, increased GRAF1 level, increased SPARC level, increased TET level, increased H19 level, increased level of TGF signaling, increased HNF level, increased PEPCK level, increased G6PC level, increased H19 level, increased PGC level, increased PGC-1a level, increased TSP level, increased TSP1 level, increased FN1 level, increased VIM level, increased COL1A1 level, increased COL3A1 level, increased COL4A1 level, increased COL5A2 level, increased HMGA2 level, increased SLUG level, increased T1MP1 level, increased α-SMA level, increased SMAD2 level, increased SMAD3 level, increased SMAD4 level, increased p-SMAD2 level, increased p-SMAD3 level, increased p-SMAD4 level, increased LIN28B level, increased NOTCH3 level, increased collagen 1 level, increased fibronectin level, increased Tet3 expression, increased Hnf4a expression, increased Pck1 expression, increased G6p expression, or any combination thereof.

In various embodiments, the method of treatment comprises monitoring the biomarker levels (e.g., RNA level or protein level or activity level, etc.) during the course of treatment of a disease or disorder. In various embodiments, the method of treatment comprises an assessment of the effectiveness of the treatment regimen for a disease or disorder, such as cancer, fibrosis, diabetes, by detecting one or more biomarkers in an effective amount from samples obtained from a subject over time and comparing the amount of biomarker or biomarkers detected. In various embodiments, a first sample is obtained prior to the subject receiving treatment and one or more subsequent samples are taken after or during treatment of the subject. In various embodiments, changes in biomarker levels over time provide an indication of effectiveness of the therapy.

To identify therapeutics or drugs that are appropriate for a specific subject, a test sample from the subject can also be exposed to a therapeutic agent or a drug, and the level of one or more biomarkers can be determined. Biomarker levels can be compared to a sample derived from the subject before and after treatment or exposure to a therapeutic agent or a drug, or can be compared to samples derived from one or more subjects who have shown improvements relative to a disease as a result of such treatment or exposure. Thus, in one aspect, the invention provides a method of assessing the efficacy of a therapy with respect to a subject comprising taking a first measurement of a biomarker panel in a first sample from the subject; effecting the therapy with respect to the subject; taking a second measurement of the biomarker panel in a second sample from the subject and comparing the first and second measurements to assess the efficacy of the therapy.

In various embodiments of the methods of the invention, the level (e.g., RNA level or protein level or activity level, etc.) of TET is determined to be increased when the level of TET in the biological sample is increased by at least 5%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level (e.g., RNA level or protein level or activity level, etc.) of TET is determined to be increased when the level of TET in the biological sample is increased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In various embodiments, TET is TET1, TET2, TET3, or any combination thereof. For example, in one embodiment, a subject is identified as having a disease or disorder associated with increased TET level, increased H19 level, increased level of TGF signaling, or any combination thereof when the TET level is increased by at least 1 fold, at least 1.2 fold, at least 1.4 fold, at least 1.6 fold, at least 1.8 fold, at least 2 fold, at least 2.2 fold, or at least 2.4 fold.

In various embodiments of the methods of the invention, the level (e.g., RNA level or protein level or activity level, etc.) of H19 is determined to be increased when the level of H19 in the biological sample is increased by at least 5%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level (e.g., RNA level or protein level or activity level, etc.) of H19 is determined to be increased when the level of H19 in the biological sample is increased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

For example, in one embodiment, a subject is identified as having a disease or disorder associated with increased TET level, increased H19 level, increased level of TGF signaling, or any combination thereof when the H19 level is increased by at least 1 fold, at least 1.2 fold, at least 1.4 fold, at least 1.6 fold, at least 1.8 fold, at least 2 fold, at least 2.2 fold, or at least 2.4 fold.

In various embodiments of the methods of the invention, the level of signaling (e.g., RNA level or protein level or activity level, etc.) of TGF is determined to be increased when the level of signaling of TGF in the biological sample is increased by at least 5%, by at least 10%, by at least 20%, by at least 30%, by at least 40%, by at least 50%, by at least 60%, by at least 70%, by at least 80%, by at least 90%, by at least 100%, by at least 125%, by at least 150%, by at least 175%, by at least 200%, by at least 250%, by at least 300%, by at least 400%, by at least 500%, by at least 600%, by at least 700%, by at least 800%, by at least 900%, by at least 1000%, by at least 1500%, by at least 2000%, by at least 2500%, by at least 3000%, by at least 4000%, or by at least 5000%, when compared with a comparator.

In various embodiments of the methods of the invention, the level signaling (e.g., RNA level or protein level or activity level, etc.) of TGF is determined to be increased when the level signaling of TGF in the biological sample is determined to be increased by at least 1 fold, at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3 fold, at least 3.5 fold, at least 4 fold, at least 4.5 fold, at least 5 fold, at least 5.5 fold, at least 6 fold, at least 6.5 fold, at least 7 fold, at least 7.5 fold, at least 8 fold, at least 8.5 fold, at least 9 fold, at least 9.5 fold, at least 10 fold, at least 11 fold, at least 12 fold, at least 13 fold, at least 14 fold, at least 15 fold, at least 20 fold, at least 25 fold, at least 30 fold, at least 40 fold, at least 50 fold, at least 75 fold, at least 100 fold, at least 200 fold, at least 250 fold, at least 500 fold, or at least 1000 fold, or at least 10000 fold, when compared with a comparator.

In one embodiment, a subject is identified as having a disease or disorder associated with increased TET level, increased H19 level, increased level of TGF signaling, or any combination thereof when the TGF signaling is increased in the biological sample as compared to a comparator. For example, in one embodiment, a subject is identified as having an enhanced risk for developing a disease or disorder associated with increased TET level, increased H19 level, increased level of TGF signaling, or any combination thereof when the TGF signaling is increased by at least 1 fold, at least 1.1 fold, at least 1.2 fold, at least 1.3 fold, at least 1.4 fold, or at least 1.5 fold.

In various embodiments, the level of one or more of markers of the invention in the biological test sample of the subject is compared with the level of the biomarker in a comparator. Non-limiting examples of comparators include, but are not limited to, a negative control, a positive control, standard control, standard value, an expected normal background value of the subject, a historical normal background value of the subject, a reference standard, a reference level, an expected normal background value of a population that the subject is a member of, or a historical normal background value of a population that the subject is a member of. In one embodiment, the comparator is a level of the one or more biomarker in a sample obtained from a subject not having a disease or disorder, such as cancer, fibrosis, and diabetes. In one embodiment, the comparator is a level of the one or more biomarker in a sample obtained from a subject known not to have a disease or disorder, such as cancer, fibrosis, and diabetes.

Additionally, therapeutic agents suitable for administration to a particular subject can be identified by detecting one or more biomarkers in an effective amount from a sample obtained from a subject and exposing the subject-derived sample to a test compound that determines the amount of the biomarker(s) in the subject-derived sample. Accordingly, treatments or therapeutic regimens for use in subjects having a disease or disorder associated with increased TET level, increased H19 level, increased level of TGF signaling, or any combination thereof can be selected based on the amounts of biomarkers in samples obtained from the subjects and compared to a comparator value. Two or more treatments or therapeutic regimens can be evaluated in parallel to determine which treatment or therapeutic regimen would be the most efficacious for use in a subject to delay onset, or slow progression of a disease. In various embodiments, a recommendation is made on whether to initiate or continue treatment of a disease.

In various exemplary embodiments, effecting a therapy comprises administering a disease-modulating drug to the subject. The subject may be treated with one or more drugs until altered levels of the measured biomarkers return closer to the baseline value measured in a population not having a disease or disorder associated with increased TET level, increased H19 level, increased level of TGF signaling, or any combination thereof, not having recurrence of a disease or disorder associated with increased TET level, increased H19 level, increased level of TGF signaling, or any combination thereof, or showing improvements in disease biomarkers as a result of treatment with a drug. Additionally, improvements related to a changed level of a biomarker or clinical parameter may be the result of treatment with a disease-modulating drug.

In one embodiment, the method comprises using a multi-dimensional non-linear algorithm to determine if the level (e.g., RNA level or protein level or activity level, etc.) of a set of biomarkers in the biological sample is statistically different than the level in a comparator sample. In various embodiments, the algorithm is drawn from the group consisting essentially of: linear or nonlinear regression algorithms; linear or nonlinear classification algorithms; ANOVA; neural network algorithms; genetic algorithms; support vector machines algorithms; hierarchical analysis or clustering algorithms; hierarchical algorithms using decision trees; kernel based machine algorithms such as kernel partial least squares algorithms, kernel matching pursuit algorithms, kernel fisher discriminate analysis algorithms, or kernel principal components analysis algorithms; Bayesian probability function algorithms; Markov Blanket algorithms; a plurality of algorithms arranged in a committee network; and forward floating search or backward floating search algorithms.

In various embodiments of the invention, the methods comprise a) providing a biological sample from the subject; b) analyzing the biological sample with an assay that specifically detects at least one biomarker of the invention in the biological sample; c) comparing the level of the at least one biomarker in the sample with the level in a comparator sample, wherein a statistically significant difference between the level of the at least one biomarker in the sample with the level in a comparator sample or earlier obtained biological sample is indicative of a development of a disease or disorder associated with increased TET level, increased H19 level, increased level of TGF signaling, or any combination thereof in the subject. In various embodiments, the methods further comprise the step of d) effectuating a treatment regimen based thereon.

In some the methods of the invention, a biological sample from a subject is assessed for the level of one or more of the markers of the invention in the biological sample obtained from the patient. The level of one or more of the markers of the invention in the biological sample can be determined by assessing the amount of polypeptide of one or more of the biomarkers of the invention in the biological sample, the amount of mRNA of one or more of the biomarkers of the invention in the biological sample, the amount of enzymatic activity of one or more of the biomarkers of the invention in the biological sample, or any combination thereof.

In one embodiment, biomarker expression includes transcription into messenger RNA (mRNA) and translation into protein. In one embodiment, the biomarker types comprise mRNA biomarkers. In various embodiments, the mRNA is detected by at least one of mass spectroscopy, PCR microarray, thermal sequencing, capillary array sequencing, solid phase sequencing, and the like. In another embodiment, the biomarker types comprise polypeptide biomarkers. In various embodiments, the polypeptide is detected by at least one of ELISA, Western blot, flow cytometry, immunofluorescence, immunohistochemistry, mass spectroscopy, and the like.

In certain embodiments, the method comprises using surgical data in combination with the detection of the relevant biomarkers described herein to establish or evaluate treatment of a disease or disorder associated with increased TET level, increased H19 level, increased level of TGF signaling, or any combination thereof. For example, in certain embodiments, the method comprises assessing the extent of primary tumor (T category), spread of a disease or disorder, such as cancer, to the lymph node (N category), or spread of a disease or disorder, such as cancer, to other parts of the body (metastatic stage) (M category). In certain embodiments, the method comprises an assessment of the Gleason score of tumor.

In one embodiment, the method comprises evaluating a treatment of a disease or disorder associated with increased TET level, increased H19 level, increased level of TGF signaling, or any combination thereof by detecting differentially expressed biomarkers in biological tissue excised from the subject during biopsy.

In one aspect, the invention contemplates the detection of differentially expressed markers using tissue microarray. In another aspect, the invention further contemplates using methods known to those skilled in the art to detect and to measure the level of differentially expressed marker expression products, such as RNA and protein, to measure the level of one or more differentially expressed marker expression products.

In one embodiment, methods of detecting or measuring gene expression utilize methods that focus on cellular components (cellular examination), or methods that focus on examining extracellular components (fluid examination). In one embodiment, a cellular or fluid examination is used to detect or measure a variety of molecules including RNA, protein, and a number of molecules that are modified as a result of the protein's function. Exemplary diagnostic methods focusing on nucleic acids include but are not limited to amplification techniques, such as PCR and RT-PCR (including quantitative variants), and hybridization techniques, such as in situ hybridization, microarrays, and blots. Exemplary diagnostic methods focusing on proteins include but are not limited to binding techniques, such as ELISA, immunohistochemistry, microarray, and functional techniques, such as enzymatic assays.

The genes identified as being differentially expressed may be assessed in a variety of nucleic acid detection assays to detect or quantify the expression level of a gene or multiple genes in a given sample. For example, traditional Northern blotting, nuclease protection, RT-PCR, microarray, and differential display methods may be used for detecting gene expression levels. Methods for assaying for mRNA include Northern blots, slot blots, dot blots, and hybridization to an ordered array of oligonucleotides. Any method for specifically and quantitatively measuring a specific protein or mRNA or DNA product can be used. However, methods and assays are most efficiently designed with array or chip hybridization-based methods for detecting the expression of a large number of genes. Any hybridization assay format may be used, including solution-based and solid support-based assay formats.

The protein products of the genes identified herein can also be assayed to determine the amount of expression. Methods for assaying for a protein include Western blot, immunoprecipitation, and radioimmunoassay. The proteins analyzed may be localized intracellularly (most commonly an application of immunohistochemistry) or extracellularly (most commonly an application of immunoassays such as ELISA).

Biological samples may be of any biological tissue or fluid. Frequently the sample will be a “clinical sample” which is a sample derived from a patient. The biological sample may contain any biological material suitable for detecting the desired biomarkers, and may comprise cellular and/or non-cellular material obtained from the individual. A biological sample can be obtained by appropriate methods, such as, by way of examples, blood draw, fluid draw, biopsy, or surgical resection. Examples of such samples include but are not limited to blood, lymph, urine, gastrointestinal fluid, semen, and biopsies. Samples that are liquid in nature are referred to herein as “bodily fluids.” Body samples may be obtained from a patient by a variety of techniques including, for example, by scraping or swabbing an area or by using a needle to aspirate bodily fluids. Methods for collecting various body samples are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, saliva, tissue or fine needle biopsy samples, tissue sample obtained during surgical resection, and archival samples with known diagnosis, treatment and/or outcome history. In certain embodiments, the biological sample comprises gastrointestinal tissue. In certain embodiments, the biological sample comprises gastrointestinal tissue of a subject having gastrointestinal cancer.

Control group samples may either be from a normal subject, samples from subjects with a known diagnosis of a disease or disorder associated with increased TET level, increased H19 level, increased level of TGF signaling, or any combination thereof, or samples from subjects with no known diagnosis of a disease or disorder associated with increased TET level, increased H19 level, increased level of TGF signaling, or any combination thereof. As described below, comparison of the expression patterns of the sample to be tested with those of the comparators can be used to assess the risk of developing a disease or disorder associated with increased TET level, increased H19 level, increased level of TGF signaling, or any combination thereof in the subject. In some instances, the control groups are only for the purposes of establishing initial cutoffs or thresholds for the assays of the invention. Therefore, in some instances, the systems and methods of the invention can evaluate a treatment of a disease or disorder associated with increased TET level, increased H19 level, increased level of TGF signaling, or any combination thereof without the need to compare with a control group.

In various embodiments, the subject is a human subject, and may be of any race, sex and age.

Information obtained from the methods of the invention described herein can be used alone, or in combination with other information (e.g., age, family history, disease status, disease history, vital signs, blood chemistry, PSA level, Gleason score, primary tumor staging, lymph node staging, metastasis staging, expression of other gene signatures relevant to outcomes of a disease or disorder, such as cancer, fibrosis, and diabetes, associated with increased TET level, increased H19 level, increased level of TGF signaling, or any combination thereof, etc.) from the subject or from the biological sample obtained from the subject.

Any drug or any combination of drugs disclosed herein may be administered to a subject to treat a disease. The drugs herein can be formulated in any number of ways, often according to various known formulations in the art or as disclosed or referenced herein.

In various embodiments, any drug or any combination of drugs disclosed herein is not administered to a subject to treat a disease. In these embodiments, the practitioner may refrain from administering the drug or any combination of drugs, may recommend that the subject not be administered the drug or any combination of drugs or may prevent the subject from being administered the drug or any combination of drugs.

In various embodiments, one or more additional drugs may be optionally administered in addition to those that are recommended or have been administered. An additional drug will typically not be any drug that is not recommended or that should be avoided.

In one embodiment, the invention includes detecting one or more mRNA biomarkers, polypeptide biomarkers, or any combination thereof in a biological sample. Biomarkers generally can be measured and detected through a variety of assays, methods and detection systems known to one of skill in the art.

Various methods include but are not limited to immunoassays, microarray, PCR, RT-PCR, refractive index spectroscopy (RI), ultra-violet spectroscopy (UV), fluorescence analysis, electrochemical analysis, radiochemical analysis, near-infrared spectroscopy (near-IR), infrared (IR) spectroscopy, nuclear magnetic resonance spectroscopy (NMR), light scattering analysis (LS), mass spectrometry, pyrolysis mass spectrometry, nephelometry, dispersive Raman spectroscopy, gas chromatography, liquid chromatography, gas chromatography combined with mass spectrometry, liquid chromatography combined with mass spectrometry, matrix-assisted laser desorption ionization-time of flight (MALDI-TOF) combined with mass spectrometry, ion spray spectroscopy combined with mass spectrometry, capillary electrophoresis, colorimetry and surface plasmon resonance (such as according to systems provided by Biacore Life Sciences). See also PCT Publications WO/2004/056456 and WO/2004/088309. In this regard, biomarkers can be measured using the above-mentioned detection methods, or other methods known to the skilled artisan. Other biomarkers can be similarly detected using reagents that are specifically designed or tailored to detect them.

Different types of biomarkers and their measurements can be combined in the compositions and methods of the present invention. In various embodiments, the protein form of the biomarkers is measured. In various embodiments, the nucleic acid form of the biomarkers is measured. In exemplary embodiments, the nucleic acid form is mRNA. In various embodiments, measurements of protein biomarkers are used in conjunction with measurements of nucleic acid biomarkers.

In various embodiments of the invention, methods of measuring polypeptide levels in a biological sample obtained from a subject include, but are not limited to, an immunochromatography assay, an immunodot assay, a Luminex assay, an ELISA assay, an ELISPOT assay, a protein microarray assay, a ligand-receptor binding assay, displacement of a ligand from a receptor assay, displacement of a ligand from a shared receptor assay, an immunostaining assay, a Western blot assay, a mass spectrophotometry assay, a radioimmunoassay (MA), a radioimmunodiffusion assay, a liquid chromatography-tandem mass spectrometry assay, an ouchterlony immunodiffusion assay, reverse phase protein microarray, a rocket immunoelectrophoresis assay, an immunohistostaining assay, an immunoprecipitation assay, a complement fixation assay, FACS, an enzyme-substrate binding assay, an enzymatic assay, an enzymatic assay employing a detectable molecule, such as a chromophore, fluorophore, or radioactive substrate, a substrate binding assay employing such a substrate, a substrate displacement assay employing such a substrate, and a protein chip assay (see also, 2007, Van Emon, Immunoassay and Other Bioanalytical Techniques, CRC Press; 2005, Wild, Immunoassay Handbook, Gulf Professional Publishing; 1996, Diamandis and Christopoulos, Immunoassay, Academic Press; 2005, Joos, Microarrays in Clinical Diagnosis, Humana Press; 2005, Hamdan and Righetti, Proteomics Today, John Wiley and Sons; 2007).

Methods for detecting a nucleic acid (e.g., mRNA), such as RT-PCR, real time PCR, microarray, branch DNA, NASBA and others, are well known in the art. Using sequence information provided by the database entries for the biomarker sequences, expression of the biomarker sequences can be detected (if present) and measured using techniques well known to one of ordinary skill in the art. For example, sequences in sequence database entries or sequences disclosed herein can be used to construct probes for detecting biomarker RNA sequences in, e.g., Northern blot hybridization analyses or methods which specifically, and, preferably, quantitatively amplify specific nucleic acid sequences. As another example, the sequences can be used to construct primers for specifically amplifying the biomarker sequences in, e.g., amplification-based detection methods such as reverse-transcription based polymerase chain reaction (RT-PCR). When alterations in gene expression are associated with gene amplification, deletion, polymorphisms and mutations, sequence comparisons in test and comparator populations can be made by comparing relative amounts of the examined DNA sequences in the test and reference cell populations. In addition to Northern blot and RT-PCR, RNA can also be measured using, for example, other target amplification methods (e.g., TMA, SDA, NASBA), signal amplification methods (e.g., bDNA), nuclease protection assays, in situ hybridization and the like.

In various embodiments, quantitative hybridization methods, such as Southern analysis, Northern analysis, or in situ hybridizations, can be used (see Current Protocols in Molecular Biology, Ausubel, F. et al., eds., John Wiley & Sons, including all supplements). A “nucleic acid probe,” as used herein, can be a DNA probe or an RNA probe. The probe can be, for example, a gene, a gene fragment (e.g., one or more exons), a vector comprising the gene, a probe or primer, etc. For representative examples of use of nucleic acid probes, see, for example, U.S. Pat. Nos. 5,288,611 and 4,851,330. The nucleic acid probe can be, for example, a full-length nucleic acid molecule, or a portion thereof, such as an oligonucleotide of at least 15, 30, 50, 100, 250 or 500 nucleotides in length and sufficient to specifically hybridize under stringent conditions to appropriate target mRNA or cDNA. The hybridization sample is maintained under conditions which are sufficient to allow specific hybridization of the nucleic acid probe to mRNA or cDNA. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe having a mRNA or cDNA in the test sample, the level of the mRNA or cDNA in the sample can be assessed. More than one nucleic acid probe can also be used concurrently in this method. Specific hybridization of any one of the nucleic acid probes is indicative of the presence of the mRNA or cDNA of interest, as described herein.

Alternatively, a peptide nucleic acid (PNA) probe can be used instead of a nucleic acid probe in the quantitative hybridization methods described herein. PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, 1994, Nielsen et al., Bioconjugate Chemistry 5:1). The PNA probe can be designed to specifically hybridize to a target nucleic acid sequence. Hybridization of the PNA probe to a nucleic acid sequence is used to determine the level of the target nucleic acid in the biological sample.

In another embodiment, arrays of oligonucleotide probes that are complementary to target nucleic acid sequences in the biological sample obtained from a subject can be used to determine the level of one or more biomarkers in the biological sample obtained from a subject. The array of oligonucleotide probes can be used to determine the level of one or more biomarkers alone, or the level of the one or more biomarkers in relation to the level of one or more other nucleic acids in the biological sample. Oligonucleotide arrays typically comprise a plurality of different oligonucleotide probes that are coupled to a surface of a substrate in different known locations. These oligonucleotide arrays, also known as “Genechips,” have been generally described in the art, for example, U.S. Pat. No. 5,143,854 and PCT patent publication Nos. WO 90/15070 and 92/10092. These arrays can generally be produced using mechanical synthesis methods or light directed synthesis methods which incorporate a combination of photolithographic methods and solid phase oligonucleotide synthesis methods. See Fodor et al., Science, 251:767-777 (1991), Pirrung et al., U.S. Pat. No. 5,143,854 (see also PCT Application No. WO 90/15070) and Fodor et al., PCT Publication No. WO 92/10092 and U.S. Pat. No. 5,424,186. Techniques for the synthesis of these arrays using mechanical synthesis methods are described in, e.g., U.S. Pat. No. 5,384,261.

After an oligonucleotide array is prepared, a nucleic acid of interest is hybridized with the array and its level is quantified. Hybridization and quantification are generally carried out by methods described herein and also in, e.g., published PCT Application Nos. WO 92/10092 and WO 95/11995, and U.S. Pat. No. 5,424,186. In brief, a target nucleic acid sequence is amplified by well-known amplification techniques, e.g., PCR. Typically, this involves the use of primer sequences that are complementary to the target nucleic acid. Asymmetric PCR techniques may also be used. Amplified target, generally incorporating a label, is then hybridized with the array under appropriate conditions. Upon completion of hybridization and washing of the array, the array is scanned to determine the quantity of hybridized nucleic acid. The hybridization data obtained from the scan is typically in the form of fluorescence intensities as a function of quantity, or relative quantity, of the target nucleic acid in the biological sample. The target nucleic acid can be hybridized to the array in combination with one or more comparators (e.g., positive control, negative control, quantity control, etc.) to improve quantification of the target nucleic acid in the sample.

The probes and primers according to the invention can be labeled directly or indirectly with a radioactive or nonradioactive compound, by methods well known to those skilled in the art, in order to obtain a detectable and/or quantifiable signal; the labeling of the primers or of the probes according to the invention is carried out with radioactive elements or with nonradioactive molecules. Among the radioactive isotopes used, mention may be made of ³²P, ³³P, ³⁵S or ³H. The nonradioactive entities are selected from ligands such as biotin, avidin, streptavidin or digoxigenin, haptenes, dyes, and luminescent agents such as radioluminescent, chemoluminescent, bioluminescent, fluorescent or phosphorescent agents.

Nucleic acids can be obtained from the cells using known techniques. Nucleic acid herein refers to RNA, including mRNA, and DNA, including cDNA. The nucleic acid can be double-stranded or single-stranded (i.e., a sense or an antisense single strand) and can be complementary to a nucleic acid encoding a polypeptide. The nucleic acid content may also be an RNA or DNA extraction performed on a biological sample, including a biological fluid and fresh or fixed tissue sample.

There are many methods known in the art for the detection and quantification of specific nucleic acid sequences and new methods are continually reported. A great majority of the known specific nucleic acid detection and quantification methods utilize nucleic acid probes in specific hybridization reactions. Preferably, the detection of hybridization to the duplex form is a Southern blot technique. In the Southern blot technique, a nucleic acid sample is separated in an agarose gel based on size (molecular weight) and affixed to a membrane, denatured, and exposed to (admixed with) the labeled nucleic acid probe under hybridizing conditions. If the labeled nucleic acid probe forms a hybrid with the nucleic acid on the blot, the label is bound to the membrane.

In the Southern blot, the nucleic acid probe is preferably labeled with a tag. That tag can be a radioactive isotope, a fluorescent dye or the other well-known materials. Another type of process for the specific detection of nucleic acids in a biological sample known in the art are the hybridization methods as exemplified by U.S. Pat. Nos. 6,159,693 and 6,270,974, and related patents. To briefly summarize one of those methods, a nucleic acid probe of at least 10 nucleotides, preferably at least 15 nucleotides, more preferably at least 25 nucleotides, having a sequence complementary to a nucleic acid of interest is hybridized in a sample, subjected to depolymerizing conditions, and the sample is treated with an ATP/luciferase system, which will luminesce if the nucleic sequence is present. In quantitative Southern blotting, the level of the nucleic acid of interest can be compared with the level of a second nucleic acid of interest, and/or to one or more comparators nucleic acids (e.g., positive control, negative control, quantity control, etc.).

Many methods useful for the detection and quantification of nucleic acid takes advantage of the polymerase chain reaction (PCR). The PCR process is well known in the art (U.S. Pat. Nos. 4,683,195, 4,683,202, and 4,800,159). To briefly summarize PCR, nucleic acid primers, complementary to opposite strands of a nucleic acid amplification target sequence, are permitted to anneal to the denatured sample. A DNA polymerase (typically heat stable) extends the DNA duplex from the hybridized primer. The process is repeated to amplify the nucleic acid target. If the nucleic acid primers do not hybridize to the sample, then there is no corresponding amplified PCR product. In this case, the PCR primer acts as a hybridization probe.

In PCR, the nucleic acid probe can be labeled with a tag as discussed elsewhere herein. Most preferably the detection of the duplex is done using at least one primer directed to the nucleic acid of interest. In yet another embodiment of PCR, the detection of the hybridized duplex comprises electrophoretic gel separation followed by dye-based visualization.

Typical hybridization and washing stringency conditions depend in part on the size (i.e., number of nucleotides in length) of the oligonucleotide probe, the base composition and monovalent and divalent cation concentrations (Ausubel et al., 1994, eds Current Protocols in Molecular Biology).

In one embodiment, the process for determining the quantitative and qualitative profile of the nucleic acid of interest according to the present invention is characterized in that the amplifications are real-time amplifications performed using a labeled probe, preferably a labeled hydrolysis-probe, capable of specifically hybridizing in stringent conditions with a segment of the nucleic acid of interest. The labeled probe is capable of emitting a detectable signal every time each amplification cycle occurs, allowing the signal obtained for each cycle to be measured.

The real-time amplification, such as real-time PCR, is well known in the art, and the various known techniques will be employed in the best way for the implementation of the present process. These techniques are performed using various categories of probes, such as hydrolysis probes, hybridization adjacent probes, or molecular beacons. The techniques employing hydrolysis probes or molecular beacons are based on the use of a fluorescence quencher/reporter system, and the hybridization adjacent probes are based on the use of fluorescence acceptor/donor molecules.

Hydrolysis probes with a fluorescence quencher/reporter system are available in the market, and are for example commercialized by the Applied Biosystems group (USA). Many fluorescent dyes may be employed, such as FAM dyes (6-carboxy-fluorescein), or any other dye phosphoramidite reagents.

Among the stringent conditions applied for any one of the hydrolysis-probes of the present invention is the Tm, which is in the range of about 65° C. to 75° C. Preferably, the Tm for any one of the hydrolysis-probes of the present invention is in the range of about 67° C. to about 70° C. Most preferably, the Tm applied for any one of the hydrolysis-probes of the present invention is about 67° C.

In one aspect, the invention includes a primer that is complementary to a nucleic acid of interest, and more particularly the primer includes 12 or more contiguous nucleotides substantially complementary to the nucleic acid of interest. Preferably, a primer featured in the invention includes a nucleotide sequence sufficiently complementary to hybridize to a nucleic acid sequence of about 12 to 25 nucleotides. More preferably, the primer differs by no more than 1, 2, or 3 nucleotides from the target flanking nucleotide sequence. In another aspect, the length of the primer can vary in length, preferably about 15 to 28 nucleotides in length (e.g., 16, 17, 18, 19, 20, 21, 22, 23, 24, 25, 26, or 27 nucleotides in length).

The concentration of the biomarker in a sample may be determined by any suitable assay. A suitable assay may include one or more of the following methods, an enzyme assay, an immunoassay, mass spectrometry, chromatography, electrophoresis or an antibody microarray, or any combination thereof. Thus, as would be understood by one skilled in the art, the system and methods of the invention may include any method known in the art to detect a biomarker in a sample.

The invention described herein also relates to methods for a multiplex analysis platform. In one embodiment, the method comprises an analytical method for multiplexing analytical measurements of markers.

In one embodiment, the method comprises administering adjuvant radiotherapy to the subject determined to be at risk for developing a disease or disorder associated with TET level, increased H19 level, increased level of TGF signaling, or any combination thereof.

### Kits

The present invention also pertains to kits useful in the methods of the invention. Such kits comprise various combinations of components useful in any of the methods described elsewhere herein, including for example, materials for quantitatively analyzing a biomarker of the invention (e.g., polypeptide and/or nucleic acid), materials for assessing the activity of a biomarker of the invention (e.g., polypeptide and/or nucleic acid), and instructional material. For example, in one embodiment, the kit comprises components useful for the quantification of a desired nucleic acid in a biological sample. In another embodiment, the kit comprises components useful for the quantification of a desired polypeptide in a biological sample. In a further embodiment, the kit comprises components useful for the assessment of the activity (e.g., enzymatic activity, substrate binding activity, etc.) of a desired polypeptide in a biological sample.

In a further embodiment, the kit comprises the components of an assay for monitoring the effectiveness of a treatment administered to a subject in need thereof, containing instructional material and the components for determining whether the level of a biomarker of the invention in a biological sample obtained from the subject is modulated during or after administration of the treatment. In various embodiments, to determine whether the level of a biomarker of the invention is modulated in a biological sample obtained from the subject, the level of the biomarker is compared with the level of at least one comparator contained in the kit, such as a positive control, a negative control, a historical control, a historical norm, or the level of another reference molecule in the biological sample. In certain embodiments, the ratio of the biomarker and a reference molecule is determined to aid in the monitoring of the treatment.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore, specifically point out the preferred embodiments of the present invention, and are not to be construed as limiting in any way the remainder of the disclosure.

### Example 1: Hepatic TET3 Contributes to Type-2 Diabetes by Inducing the HNF4α Fetal Isoform

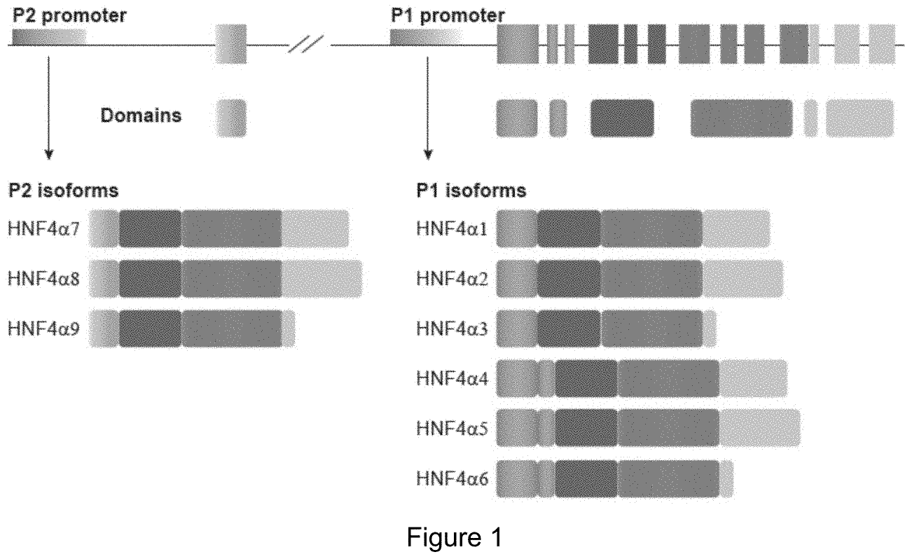

The evolutionarily conserved, liver-enriched transcription factor HNF4α is essential for hepatic development and function (Walesky C et al., 2015, Gene Expres 16:101-108). While studies have focused on the P2-to-P1 promoter switch during hepatic differentiation (Torres-Padilla M E et al., 2001, Mech Dev 109:183-193; Briancon N. et al., 2004, J Biol Chem 279:33398-33408; Ancey P B et al., 2017, Stem Cell Reports 9:264-278), none has yet documented P2 promoter reactivation in adult liver under physiological/pathological conditions. Mutations in HNF4α that impair pancreatic β-cell function have been implicated in maturity-onset diabetes of the young 1 (MODY1, a rare, inherited form of diabetes) (Ryffel G U, 2001, J Mol Endocrinol 27:11-29). Notably, single nucleotide polymorphisms (SNPs) in the P2 promoter have been associated with increased risk of T2D but via unclear mechanisms (Muller Y L et al., 2005, Diabetes 54:3035-3039; Mohlke K L et al., 2005, Curr Diab Rep, 5:149-156). Multiple HNF4α isoforms (α1-α9) are expressed by alternative splicing in a development- and tissue-specific manner (FIG. 1)(Torres-Padilla M E et al., 2001, Mech Dev 109:183-193; Briancon N. et al., 2004, J Biol Chem 279:33398-33408). The P2-derived isoforms (P2 isoform herein) differ from P1-derived isoforms (P1 isoform herein) only in their N-terminal regions. The α7/α8 isoform is predominantly expressed in the fetal liver, whereas the α1/α2 isoform predominates in the adult liver (Torres-Padilla M E et al., 2001, Mech Dev 109:183-193; Briancon N. et al., 2004, J Biol Chem 279:33398-33408; Ancey P B et al., 2017, Stem Cell Reports 9:264-278).

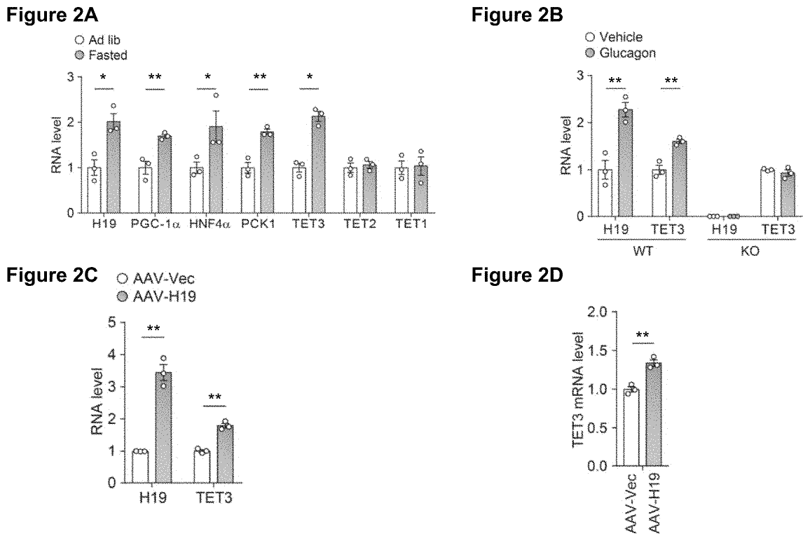

During fasting, hepatic expression of HNF4α and its transcriptional coactivator PGC-1a both increase and transcriptionally activate the rate-limiting enzymes PEPCK and G6PC (encoded by Pck1 and G6pc, respectively), leading to gluconeogenesis (Rhee J et al, 2003, Proc Natl Acad Sci USA 100:4012-4017; Yoon J C et al., 2001, Nature 413:131-138; Sharabi K et al., 2017, Cell 169:148-160). The present invention, in part, demonstrates that fasting upregulates H19 long noncoding RNA in the liver contributing to increased expression of HNF4α (Zhang N et al., 2018, JCI Insight 3:e120304). While studying the role of H19 in HGP, fasting increased levels of H19 were observed as well as mRNAs for PGC-1α, HNF4α, and PEPCK. Curiously, fasting also increased mRNA expression of TET3, but not of its family members TET2 and TET1 (FIG. 2A). TETs belong to a new class of DNA demethylases that oxidize 5-methylcytosine (5mC) to generate 5-hydroxymethylcytosine (5hmC), which is subsequently converted to unmethylated cytosine (Rasmussen K D et al., 2016, Genes Dev 30:733-750; An J et al., 2017, Exp Mol Med 49:e323; Liu R et al., 2013, Circulation 128:2047-2057). The enzymatic activity of TETs is regulated by co-factors including α-ketoglutarate (α-KG) generated through the TCA cycle and vitamin C, and by post-translational modifications (Etchegaray J P et al., 2016, Mol Cell 62:695-711). While much is known about the role of TETs in DNA methylation, development, stem cells, and cancer (Rasmussen K D et al., 2016, Genes Dev 30:733-750; An J et al., 2017, Exp Mol Med 49:e323; Liu R et al., 2013, Circulation 128:2047-2057), little is known about their role in glucose metabolism.

Glucagon induces H19 expression in hepatic cells (Zhang N et al., 2018, JCI Insight 3:e120304) and H19 positively regulates TET3 expression (Cao T et al., 2019, Oncogene In press). Primary hepatocytes from wild-type (WT) and H19 knockout (KO) mice (Zhang N et al., 2018, JCI Insight 3:e120304; Geng T et al., 2018, Diabetes 67:2183-2198) were stimulated with glucagon. In WT hepatocytes, H19 expression was readily induced by glucagon, as was Tet3; however, in KO hepatocytes, glucagon no longer stimulated Tet3 expression (FIG. 2B). Next, H19 was expressed in WT primary hepatocytes with an adeno associated virus-based vector (AAV-H19, Zhang N et al., 2018, JCI Insight 3:e120304) in absence of glucagon. Exogenous H19 expression increased TET3 mRNA levels (FIG. 2C). Consistently, livers from ad libitum fed mice injected with AAV-H19 showed increased Tet3 mRNA (FIG. 2D). Thus, H19 promotes glucagon-induced TET3 upregulation in isolated hepatocytes.

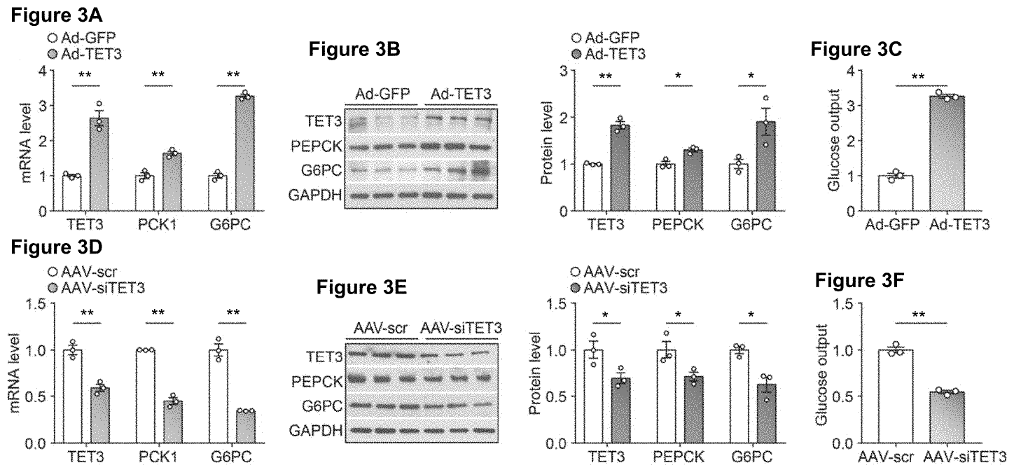

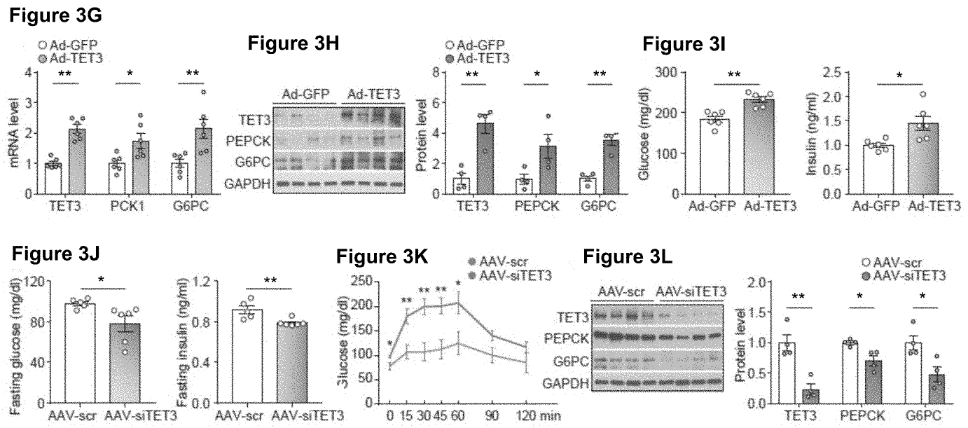

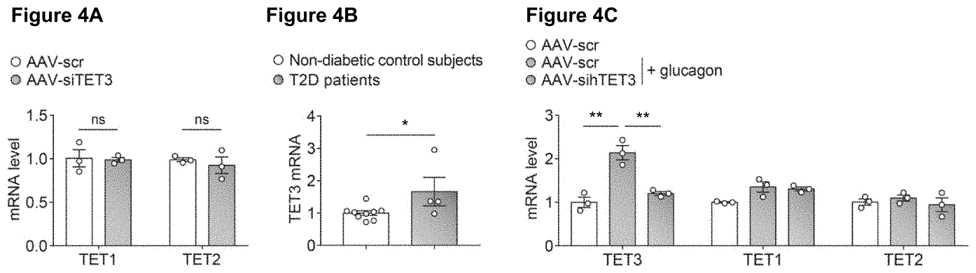

To determine whether Tet3 expression in the absence of upstream stimulators (glucagon and H19) is sufficient to enhance glucose production, TET3 was expressed in primary hepatocytes from H19 KO mice. Hepatocytes were infected with viruses containing a cDNA encoding TET3 (Ad-TET3) or green fluorescent protein (Ad-GFP). When TET3 was overexpressed, increased expression of Pck1 and G6pc was evident (FIG. 3A and FIG. 3B). TET3 overexpression also increased glucose production (FIG. 3C). In contrast, when WT hepatocytes were infected with AAV-siTET3 (specific against mouse TET3, or a non-targeting siRNA control AAV-scr) in the presence of glucagon stimulation, it led to decreased expression of Pck1 and G6pc (FIG. 3D and FIG. 3E) and glucose production (FIG. 3F). TET3 knockdown did not affect the expression of TET2 and TET1 (FIG. 4A), confirming the specificity of the TET3 siRNA. Thus, TET3 augments glucose production, at least partially by increasing expression of key gluconeogenic genes in isolated hepatocytes.

To demonstrate that TET3 regulates HGP in vivo, Ad-TET3 (or Ad-GFP) viruses were infused through the tail vein into H19 KO mice. Systemic administration of recombinant adenoviruses into rodents resulted in expression of transgenes in the liver, with no detectable expression in other peripheral tissues and brain; nor was hepatotoxicity detected (Yoon J M et al., 2001, Nature 413:131-138; Trinh K Y et al., 1998, J Biol Chem 273:31615-31620; O'Doherty R M et al., 1999, Diabetes 48:2022-2027). Following 10 days of viral administration, mice fed ad libitum were sacrificed and liver and blood samples were harvested. Mice infused with Ad-TET3 had a significant increase in hepatic TET3 expression relative to mice infused with Ad-GFP, which was accompanied by increased expression of Pck1 and G6pc (FIG. 3G and FIG. 3H); there was also an increase in blood glucose and insulin levels (FIG. 3I).

To determine whether upregulation of TET3 is necessary for HGP during fasting, AAV-siTET3 or AAV-scr were injected via tail vein into WT mice followed by fasting 10 days later. Systemic infusion of recombinant AAVs into mice leads to liver-specific expression of transgenes (Zhang N et al., 2018, JCI Insight 3:e120304). Mice infused with AAV-siTET3 showed a significant decrease in fasting blood glucose and fasting insulin, as compared to AAV-scr infused animals (FIG. 3J). Pyruvate tolerance tests (PTT, a readout for HGP) showed lower glucose levels following pyruvate injection (FIG. 3K). Protein analyses revealed decreased levels of TET3, PEPCK and G6PC in livers of AAV96 siTET3 relative to AAV-scr injected animals (FIG. 3L).

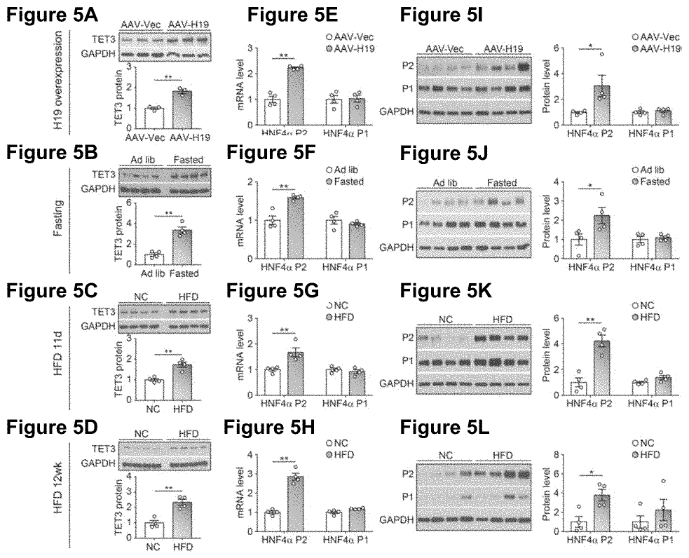

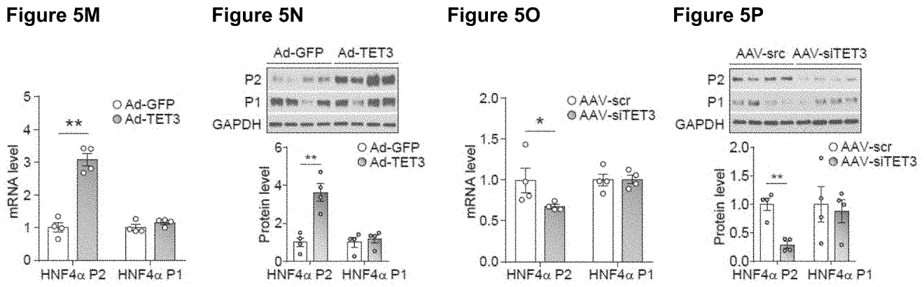

Increased H19 expression was detected in the liver during fasting and in livers of human and mouse with T2D (Zhang N et al., 2018, JCI Insight 3:e120304; Nilsson E et al., 2015, J Clin Endocrinol Metab 100:E1491-1501) conditions known to have physiological and pathological increase in gluconeogenesis, respectively. As H19 positively regulates Tet3 expression, elevated TET3 was evident in all conditions (FIG. 5A through FIG. 5D) where H19 expression was increased (Zhang N et al., 2018, JCI Insight 3:e120304). Importantly, mining of the human diabetic liver database (Pihlajamaki J et al., 2009, J Clin Endocrinol Metab 94:3521-3529) revealed a significant increase in expression of TET3 in the liver of T2D patients as compared to non-diabetic controls (FIG. 4B). Together, these results suggest that H19/TET3-mediated regulation of HGP is likely conserved between human and mouse.

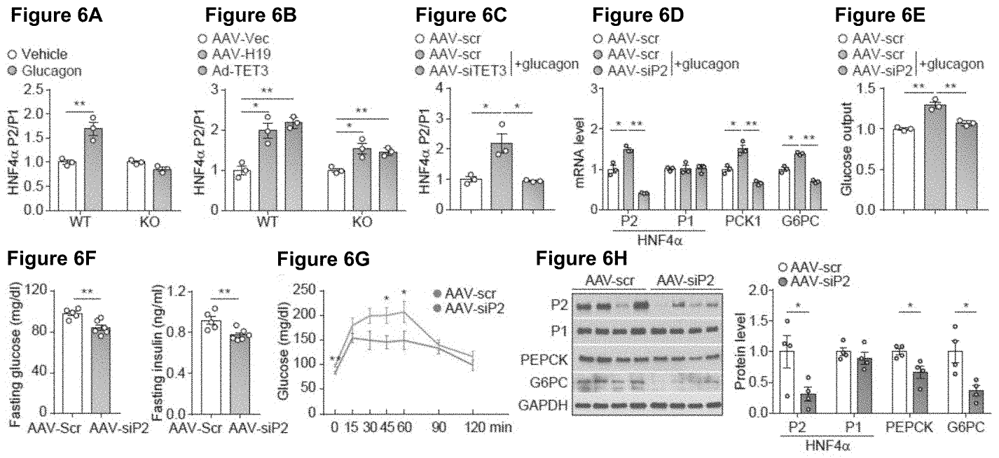

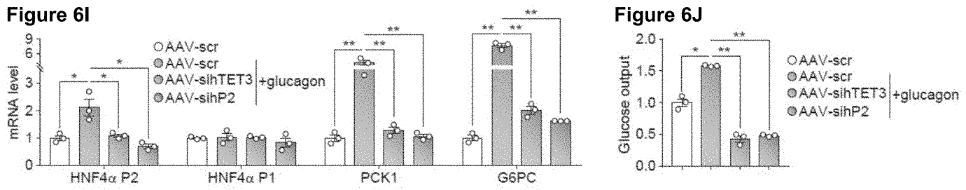

Remarkably, increased Tet3 expression also led to an increase in expression of P2- (but not P1) specific HNF4α isoform at levels of both mRNA (FIG. 5E through FIG. 5H) and protein (FIG. 5I through FIG. 5L). Although not bound by any particular theory, these data suggest the hypothesis that TET3-mediated reactivation of HNF4α P2 promoter and the derived isoform may reflect a previously unexpected mechanism of gluconeogenesis activation in adult liver. TET3 overexpression (FIG. 3G and FIG. 3H) increased P2 isoform at both the mRNA (FIG. 5M) and protein (FIG. 5N) levels without affecting P1 isoform levels. In contrast, TET3 knockdown (FIG. 3L) selectively decreased the P2 isoform (FIG. 5O and FIG. 5P). Next, primary mouse hepatocytes were used to test effects of glucagon and H19 on P2 and P1 promoter usage. While glucagon increased P2 usage in WT hepatocytes, it failed to do so in H19 KO hepatocytes (FIG. 6A). On the other hand, without glucagon exogeneous expression of either H19 or TET3 alone was sufficient to increase P2 usage in both WT and KO hepatocytes (FIG. 6B). Further, when TET3 was downregulated by siRNA, glucagon no longer induced P2 (FIG. 6C). Together, these results show that glucagon selectively induces the P2 isoform and that TET3 is required for this induction to occur in hepatocytes.

To address whether an increase in the P2 isoform is necessary for enhanced glucose production, effects of P2-specific siRNA (AAV-siP2) knockdown on gluconeogenic gene expression were tested. As seen in FIG. 6D, glucagon expectedly increased the P2 (but not P1) isoform in hepatocytes; it also increased expression of Pck1 and G6pc. Infection with AAV-siP2 downregulated the P2 isoform without affecting the P1 isoform (FIG. 6D). Importantly, knockdown of the P2 isoform not only decreased Pck1 and G6pc expression (FIG. 6D), but also abolished glucagon-induced glucose production (FIG. 6E). Thus, the P2 isoform is critical for glucagon induced glucose production in isolated hepatocytes. To determine whether this regulation also occurs in vivo, mice were injected with AAV-scr or AAV-siP2 viruses. Ten days later, mice injected with AAV-siP2 showed decreased fasting glucose and fasting insulin (FIG. 6F) and decreased PTT (FIG. 6G), as compared to AAV-scr injected animals. Protein analysis confirmed selective decrease in the P2 isoform, with a concomitant decrease in PEPCK and G6PC (FIG. 6H).

Next, primary human hepatocytes were infected with AAV-scr, AAV-sihTET3 (siRNA against human TET3), or AAV-sihP2 (siRNA specific for human HNF4α P2) and treated with vehicle or glucagon. Glucagon stimulated expression from P2 (but not P1) and also increased expression of PCK1 and G6PC; knockdown of either TET3 or the P2 isoform was sufficient to reduce PCK1 and G6PC mRNAs to the control levels (FIG. 6I). Knockdown of TET3 or the HNF4α P2 isoform also led to decreased glucose production (FIG. 6J). The lower than basal level of glucose production in the TET3 or P2 isoform knockdown group reflected persistent siRNA effects. Neither TET1 nor TET2 expression was affected by AAV-sihTET3 (FIG. 4C) and the P1 isoform level was not altered by AAV-sihP2 (FIG. 6I). Based on these data the mechanism of TET3-mediated HNF4α P2 isoform induction in HGP is likely conserved between human and mouse.

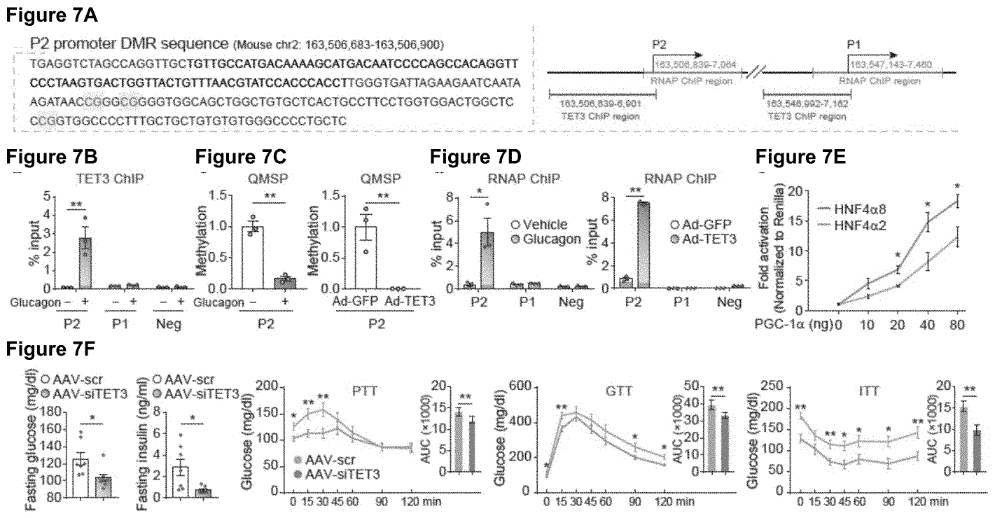

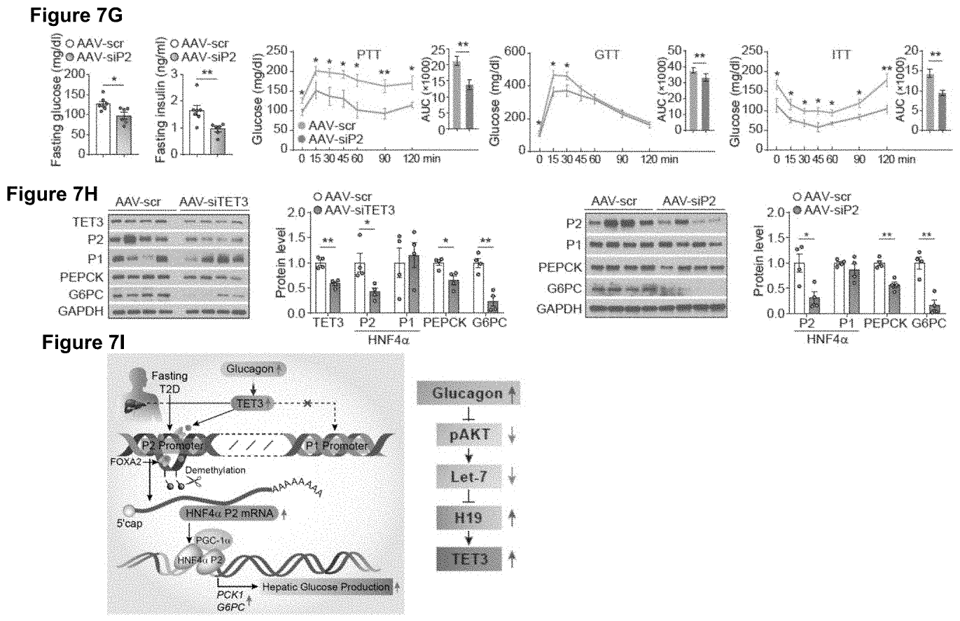

Previous studies showed a positive correlation between increased H19 expression and Hnf4a promoter hypomethylation in human hepatoma cells and in livers of multiple mouse models, including fasting, high fat diet (HFD)-induced T2D, and liver-specific H19 overexpression 13. The differentially methylated region (DMR) mapped to the Hnf4a P2 promoter known to be highly conserved between human and mouse (FIG. 7A, left) (Zhang N et al., 2018, JCI Insight 3:e120304). Although bot bound by any particular theory, as Tet3 expression was also increased under these conditions (FIG. 5A through FIG. 5D), it was hypothesized that TET3 may activate P2-specific transcription by binding and demethylating P2 promoter. TETs bind and demethylate DNA leading to transcription activation (An J et al., 2017, Exp Mol Med 49:e323; Liu R et al., 2013, Circulation 128:2047-2057; Cao T et al., 2019, Oncogene In press; Wu H et al., 2011, Genes Dev 25:2436-2452). First, it was demonstrated that glucagon promotes TET3 binding to P2 promoter. Mouse hepatocytes were treated with glucagon or vehicle, followed by chromatin immunoprecipitation coupled with qPCR (ChIP-qPCR). A TET3-specific antibody (Cao T et al., 2019, Oncogene In press) was used to immunoprecipitate protein-DNA complexes from hepatocytes and qPCR amplified the P2 and P1 promoters (FIG. 7A, right). Glucagon stimulation dramatically increased binding of TET3 to P2 but not P1 or a negative control region (Zhou J et al., 2015, Nat Commun 6:10221) (FIG. 7B). To address whether increased binding leads to P2 promoter hypomethylation, quantitative methylation-specific PCR (QMSP) was performed using the previously described methods (Cao T et al., 2019, Oncogene In press; Zhou J et al., 2015, Nat Commun 6:10221; Zhong T et al., 2016, Oncogene 36:2345-2354). The QMSP primers were designed based on the differentially methylated cytosine residues within the DMR of P2 (FIG. 7A, left). FIG. 7C shows that treatment of hepatocytes with glucagon significantly decreased methylation of P2. Exogenous TET3 expression in H19 KO hepatocytes in the absence of glucagon also led to hypomethylation of P2. To determine whether P2 promoter demethylation contributes to increased transcription, ChIP assays were performed using anti-Ser-5(P)-RNP antibody that specifically recognizes activated RNA polymerase (RNAP)(Zhou J et al., 2015, Nat Commun 6:10221). The binding of activated RNAP to Hnf4a between glucagon and vehicle treated hepatocytes was compared to assess relative transcription activity of both P2 and P1 promoters (FIG. 7A, right). An approximately 5-fold enrichment in RNAP at the P2 promoter in glucagon-stimulated versus vehicle treated hepatocytes and no change in RNAP association with P1 was observed (FIG. 7D). Similar results were obtained when TET3 was overexpressed in H19 KO hepatocytes in the absence of glucagon (FIG. 7D).

As the P2 isoform is specifically induced during fasting, it was hypothesized that this isoform is more potent than the P1 isoform in transactivation of gluconeogenic genes. Transcription activation of Pck1 and G6pc by HNF4α requires PGC-1a which by itself does not bind DNA (Rhee J et al., 2003, Proc Natl Acad Sci USA, 100:4012-4017; Sharabi K et al., 2017, Cell 169:148-160). A luciferase reporter (gAF1, Sharabi K et al., 2017, Cell 169:148-160) driven by a fragment of the Pck1 promoter harboring a HNF4α binding site was used. Plasmids expressing human HNF4α8 (representing a P2-derived isoform) (Erdmann S et al., 2007, Biol Chem 388:91-106) or HNF4α2 (representing a P1-derived isoform) (Thomas H et al., 2004, Nucleic Acids Res 32:e150) were transfected into U-2 OS cells, together with gAF1, a Renilla luciferase reporter for transfection normalization (Sharabi K et al., 2017, Cell 169:148-160), and increasing amounts of a PGC-1a expression vector (Lerin C et al., 2006, Cell Metab 3:429-438). The transcriptional activity of HNF4α8 was significantly higher than that of HNF4α2 in increasing concentrations of PGC-1a (FIG. 7E), although HNF4α8 and HNF4α2 were expressed at equivalent levels. Thus, the P2 isoform, when co-activated by PGC-1α, is a more potent transactivator of gluconeogenic genes.

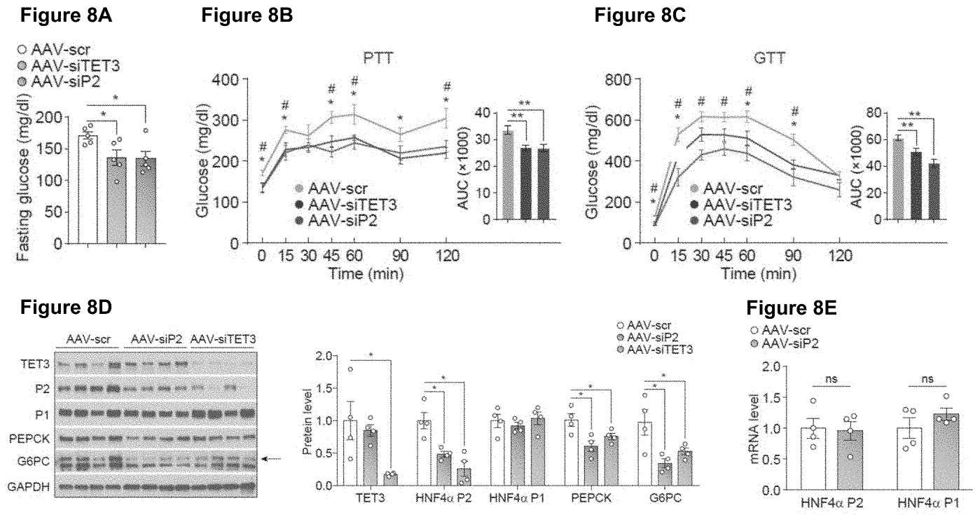

Excessive HGP in T2D is primarily a result of dysregulated gluconeogenesis, a major contributor to impaired glucose homeostasis (Magnusso I et al., 1992, J Clin Invest 90:1323-1327). Mice with T2D exhibit increased hepatic expression of TET3 and HNF4α P2 isoform (FIG. 5). Human T2D patients showed increased TET3 expression in the liver (FIG. 4B). Experiments were then conducted to assess whether inhibition of TET3 or the P2 isoform would reduce HGP thereby improving glucose homeostasis, using both HFD and genetic (Lep^{ob/ob}) mouse models of T2D (Sharabi K et al., 2017, Cell 169:148-160). The diabetic mice were infused with AAV-scr, AAV-siTET3, or AAV-siP2, and the effects of gene knockdown on glucose metabolism were assessed 10 days later. In the HFD animals, knockdown of TET3 significantly decreased fasting blood glucose, fasting insulin, and PTT (FIG. 7F), suggesting decreased hepatic gluconeogenesis, which was further supported by decreased protein levels of HNF4α P2, PEPCK and G6PC in the liver (FIG. 7H). Glucose tolerance tests (GTT) and insulin tolerance tests (ITT) showed significantly enhanced glucose tolerance and insulin sensitivity in TET3 knockdown as compared to control animals (FIG. 7F). Effects of HNF4α P2 knockdown mirrored those of TET3 knockdown (FIG. 7G and FIG. 7H). Similar observations were made using Lep^{ob/ob} mice (FIG. 8A through FIG. 8D). While in the adult pancreas both P2 and P1 promoters of HNF4α were reported to be active and important for pancreas β-cell function (Eeckhoute J et al., 2003, Endocrinology 144:1686-1694), the unchanged expression of P2 and P1 isoforms in mice infected with AAV190 siP2 (FIG. 8E) ruled out the possibility that the decreased fasting blood insulin was caused by altered HNF4α in pancreas.

In summary (FIG. 7I), this is the first report of HNF4α P2 promoter reactivation in adult liver and its critical role in control of HGP under both physiological and pathological conditions. The regulation involves an epigenetic mechanism mediated by a member of the TET family proteins not previously known to have a role in glucose homeostasis. Further, this TET3-mediated P2 promoter reactivation in HGP appears to be conserved between human and mouse. Finally, the inhibition of TET3 or only the P2-specific isoform was shown to alleviate T2D in mouse models. The present invention provides in part a mechanistic link between the P2 promoter SNPs and increased risk of T2D (Muller Y L et al., 2005, Diabetes 54:3035-3039; Mohlke K L et al., 2005, Curr Diab Rep 5:149-156) and suggests that targeting TET3, the P2-specific isoform, or both, have therapeutic potential for T2D.

### Example 2: TET3-Mediated DNA Demethylation Regulates TGF-β Signaling Linked to Fibrosis

This invention focuses in part on the use of cell culture and mouse models to demonstrate that TET3 is a novel activator of TGF-β signaling that simultaneously increases expression of multiple key TGF-β pathway genes at multiple levels (the ligand, the receptor and the activator) via an epigenetic mechanism. The present invention also shows that TGF-β1 stimulates TET3 expression. This invention in part also described a double-positive feedback mechanism involving TET3 and TGF-β1 in liver fibrosis, which may be pertinent to fibrotic diseases in other organs.

### TET3 Expression Increases in Fibrotic Liver

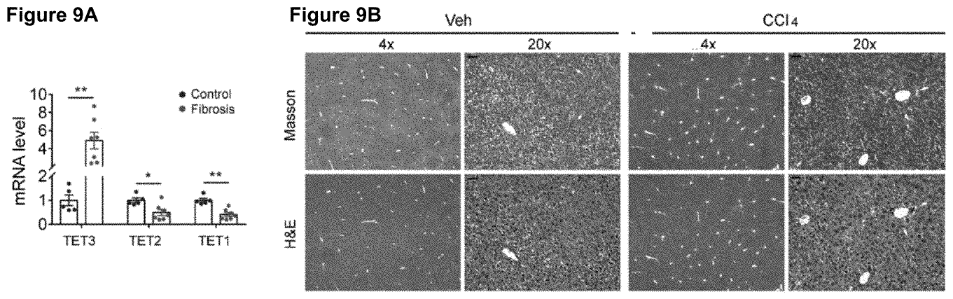

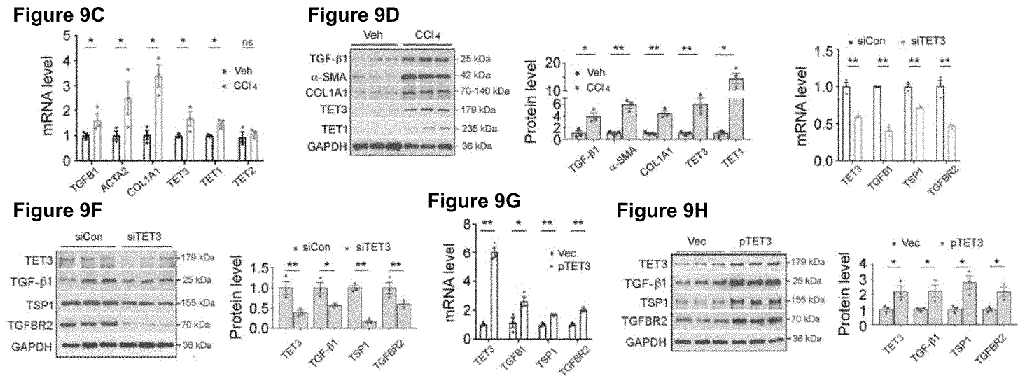

TET expression in fibrotic and non-fibrotic liver tissues was analyzed. First, liver tissues were obtained from 12 patients who underwent liver cancer resection. All patients had a history of chronic hepatitis B viral infection. Among the 12 patients, 7 were diagnosed with liver cancer with concurrent liver fibrosis (fibrosis group) and the other 5 were diagnosed with liver cancer without liver fibrosis (control group). Fibrotic liver tissues were collected from cancer-free background livers in the fibrosis group; non-fibrotic liver tissues were collected from cancer-free background livers in the control group. While the mRNA expression of TET3 was significantly increased in the fibrotic versus the non-fibrotic livers, that of TET2 and TET1 was decreased (FIG. 9A). Next, mice were repetitively exposed to carbon tetrachloride (CCl4) to induce liver fibrosis, a well-established experimental model primarily involving the activation of HSCs (Montosi G et al., 1998, Am J Pathol 152:1319-1326). Hematoxylin and eosin (H&E) and Masson's trichrome staining assays confirmed fibrotic changes in the CCl4-treated livers (FIG. 9B). Reverse transcription and quantitative PCR (qPCR) (FIG. 9C) and immunoblotting (IB) (FIG. 9D) analyses of liver tissues revealed increased expression of fibrotic markers TGF-β1 (TGFB1), α-SMA (ACTA2), and COL1A1 (COL1A1) in CCl4-treated as compared to vehicle-treated livers. The expression of TET3 and TET1 (but not TET2) also increased in the fibrotic livers (FIG. 9C and FIG. 9D). As TET3 expression is consistently increased both in human and mouse fibrotic livers, following studies focused on TET3.

TET3 Promotes Expression of Key TGF-β Signaling Genes

Experiments were also conducted to assess whether TET3 regulates expression of key TGF-β pathway genes TGFB1, TSP1, and TGFBR2 in liver cells. Thus, siRNA-mediated TET3 knockdown experiments were performed in human hepatic stellate cell line LX-2 and effects on expression of TGFB1, TSP1, and TGFBR2 were analyzed. Downregulation of TET3 decreased expression of TGFB1, TSP1 and TGFBR2 at levels of both mRNA (FIG. 9E) and protein (FIG. 9F). Conversely, when TET3 was overexpressed from a human TET3 expression vector pTET3 (Ko Metal., 2013, Nature 497:122-126), the expression of TGFB1, TSP1, and TGFBR2 increased (FIG. 9G and FIG. 9H). These results suggest that TET3 positively regulates expression of TGFB1, TSP1, and TGFBR2 in HSCs.

TET3 Epigenetically Regulates Target Gene Expression

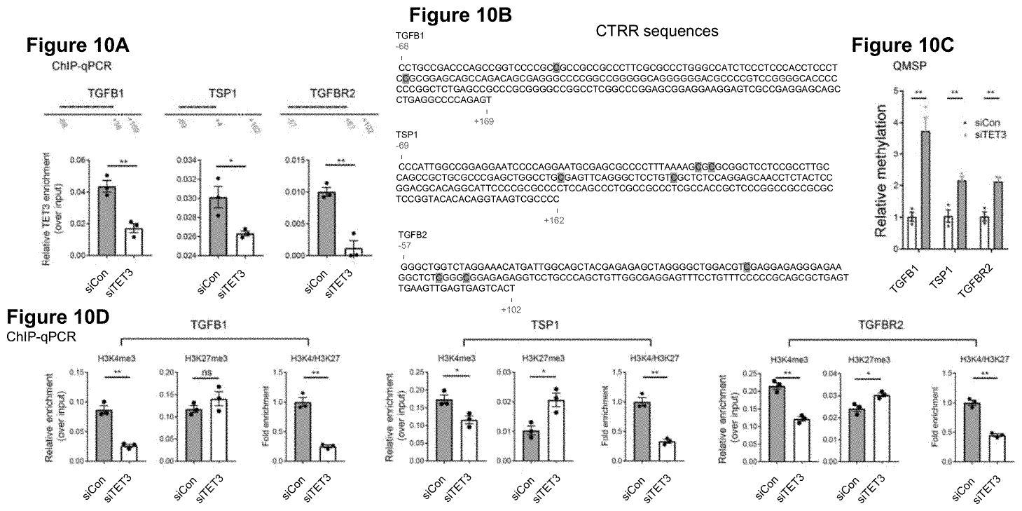

As DNA-binding proteins, TETs activate gene transcription by promoting DNA demethylation (Liu R et al., 2013, Circulation 128:2047-2057; An J et al., 2017, Exp Mol Med 49:e323; Wu H et al., 2011, Genes Dev 25:2436-2352). First, tests were conducted to assess whether there are direct interactions between TET3 and its target genes. Thus, chromatin immunoprecipitation coupled with qPCR (ChIP-qPCR) experiments were carried out. A TET3-specific antibody was used to immunoprecipitate protein-DNA complexes from LX-2 cells transfected with a TET3 siRNA (siTET3) or a non-targeting control siRNA (siCon) and qPCR amplified the critical transcriptional regulatory regions (CTRRs) of the TGFB1 (Kim S J et al., 1989, J Biol Chem 264:402-408), TSP1 (Stenina-Adognravi O, 2014, Matrix Biol 37:69-82) and TGFBR2 (Yamashita S et al., 2008, Cancer Res 68:2112-2121) promoters. In TET3 knockdown cells, binding of TET3 to the respective promoters was significantly reduced as compared to control cells, consistent with physical interactions of TET3 with these promoters (FIG. 10A).

Next, studies were conducted to determine whether TET3 knockdown alters promoter methylation of TGFB1, TSP1 and TGFBR2. The genome-wide single-nucleotide resolution DNA methylation studies from human uterine cells following TET3 knockdown showed increased methylation in the CTRRs of the TGFB1, TSP1 and TGFBR2 promoters (FIG. 10B). To determine whether TET3 knockdown in LX-2 cells leads to increased methylation of the CTRRs of TGFB1, TSP1 and TGFBR2, quantitative methylation-specific PCR (QMSP) was performed using the previously described methods (Zhou J et al., 2015, Nat Commun 6:10221; Zhong T et al., 2016, Oncogene 36:2345-2354). The QMSP primers were designed based on the differentially methylated cytosine residues within the CTRRs (FIG. 10B, green highlighted cytosines). As seen in FIG. 10C, cells treated with siTET3 had an increase in methylation in the CTRRs as compared with cells treated with siCon. These results suggest that binding of TET3 to TGFB1, TSP1 and TGFBR2 induces promoter demethylation.

To test whether TET3 knockdown affects chromatin states of the genes, LX-2 cells were transfected with siCon or siTET3, followed by ChIP-qPCR, immunoprecipitating with antibodies specific for the H3K4me3 (active) or H3K27me3 (inactive) marks and amplifying the CTRRs of TGFB1, TSP1 and TGFBR2. ChIP analysis showed that TET3 knockdown significantly decreased H3K4me3 (FIG. 10D, left columns) and increased H3K27me3 (middle columns) association with all three promoters, such that the ratios of H3K4me3/H3K27me3 decreased significantly (right columns). These results imply that TET3 knockdown promotes a heterochromatin conformation, diminishing chromatin accessibility at the TGFB1, TSP1 and TGFBR2 promoters.

TGF-β1 Upregulates TET3 and Promotes TGF-β Signaling in a TET3-Dependent Manner

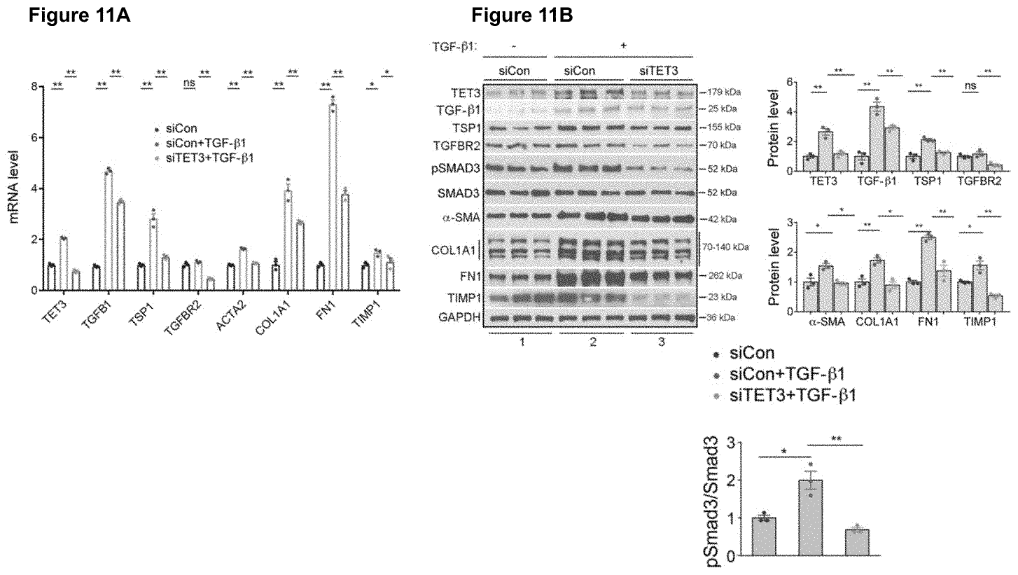

Given that TET3 promotes expression of key TGF-β signaling genes TGFB1, TSP1 and TGFBR2 (FIG. 9), further studies were conducted to address whether increased TGF-β signaling is mediated by TET3. Thus, LX-2 cells were stimulated with TGF-β with or without TET3 siRNA knockdown, followed by assessment of TGF-β signaling and profibrotic gene expression. TGF-β stimulation upregulated TET3 at levels of both mRNA (FIG. 11A) and protein (FIG. 11B). The expression of TGF-β1, TSP1, and TGFBR2 also increased, with concomitant increase in SMAD3 phosphorylation (marker for TGF-β signaling activation) and expression of α-SMA, COL1A1, FN1 and TIMP1 (markers for fibrotic activation) (FIG. 11A and FIG. 11B). However, when cells were stimulated with TGF-β1 while TET3 was downregulated by siRNA, the effects were no longer observed (FIG. 11A and FIG. 11B). Taken together with results demonstrating a positive regulation of TGFB1 expression by TET3 (FIG. 9), these data suggest a double-positive feedback loop between TGF-β1 and TET3 in regulation of TGF-β signaling and profibrotic gene expression.

LIN28B/Let-7 Axis Contributes to TGF-β-Induced Upregulation of TET3

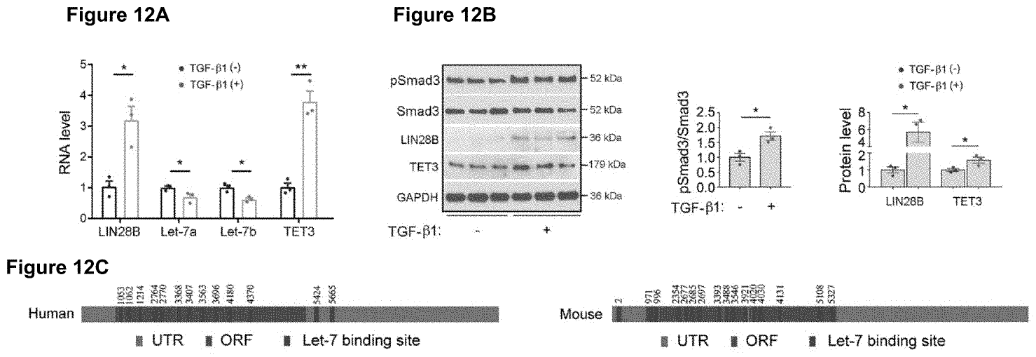

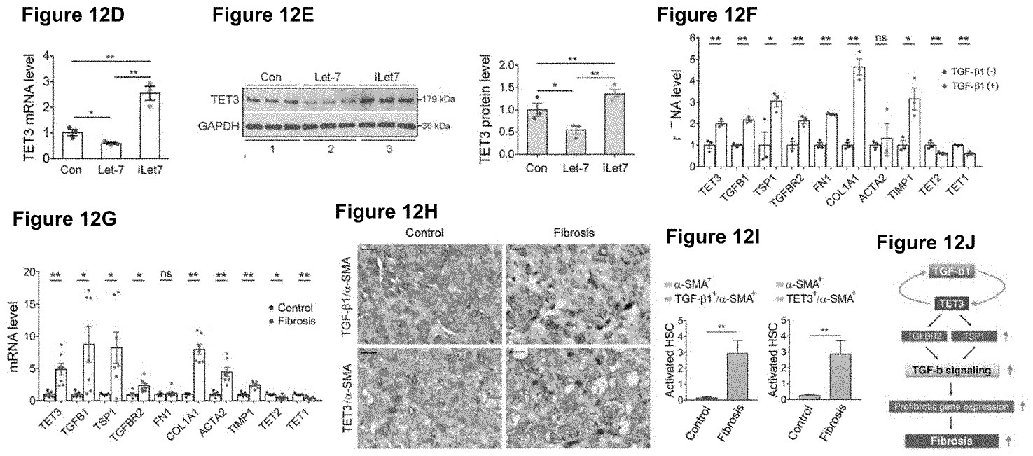

Previous studies have shown that in mouse mesangial cells TGF-β1 transcriptionally stimulates LIN28B expression by inducing Smad2/3 binding to a Smad-responsive element within the LIN28B promoter (Park J T et al., 2014, Am J Physiol Renal Physiol 307:F1390-1403). Increased LIN28B expression led to decreased production of microRNA let-7 (Park J T et al., 2014, Am J Physiol Renal Physiol 307:F1390-1403), as LIN28B is known to inhibit let-7 biogenesis by blocking processing from let-7 precursors (Huang Y et al., 2012, Wiley Interdiscip Rev RNA). Notably, Lin28b deletion mice exhibited decreased hepatic stellate cell activation and liver fibrosis following alcoholic liver injury (McDaniel K et al., 2017, J Biol Chem 292:11336-11347), suggesting a link between the LIN28B/let-7 axis, HSC activation, and liver fibrosis. To determine whether LIN28B, in conjunction with let-7, mediates TGF-β1-induced TET3 upregulation, LX-2 cells were treated with TGF-β1. TGF-β1 stimulated expression of LIN28B, which was accompanied by decreased expression of let-7a and let-7b (FIG. 12A) and increased expression of TET3. At the protein level, TGF-β1 increased Smad3 phosphorylation (indication for activation of TGF-β signaling) as well as expression of LIN28B and TET3 (FIG. 12B). Collectively, these results suggest that LIN28B positively regulates TET3 expression by decreasing let-7 production.

MicroRNAs inhibit gene expression by binding to complementary sequences in target mRNAs, inducing mRNA degradation and translational repression (Fabian M R et al., 2012, Nat Struct Mol Biol 19:586-593). Bioinformatics analysis was performed and predicted multiple let-7-binding sites in both human and mouse TET3 mRNAs, with binding sites concentrated in the open reading frame (ORF) (FIG. 12C). To test whether TET3 is a target of let-7-mediated inhibition, let-7 or a let-7-specific inhibitor (iLet7) (Ghazal S et al., 2015, Mol Med 7:996-1003; Zuckerwise L et al., 2016 Oncotarget 7:38398-38407) were transfected into LX-2 cells and TET3 expression was examined. It was expected that increasing intracellular concentration of let-7 by transfecting exogeneous let-7 would decrease expression of let-7 target genes, as compared to control miRNA (Con) transfected cells. iLet7 are chemically modified, single-stranded nucleic acids that bind to let-7 specifically and block its activity. Thus, transfecting iLet7 would sequester endogenous let-7, whereby relieving repression of target genes by let-7. As seen in FIG. 12D, TET3 mRNA was downregulated in let-7 transfected cells (middle bar) and upregulated in iLet7 transfected cells (right bar), as compared to control miRNA transfected cells (left bar). Similar results were obtained when proteins were analyzed (FIG. 12E). These data suggest that TET3 is likely a target of let-7-mediated posttranscriptional regulation and that the LIN28B/let-7 pathway likely contributes to TET3 upregulation by TGF-β1 in HSCs.

TGF-β1 and TET3 Form a Double-Positive Feedback Loop Promoting Profibrotic Gene Expression

The newly identified signal transduction pathway was tested in primary human HSCs and human liver tissue samples. RNAs were extracted from primary human HSCs treated with TGF-β1 or vehicle, followed by qPCR analysis of key pathway genes. In line with findings from LX-2 cells, increased expression of TET3, TGFB1, TSP1, TGFBR2, FN1, and COL1A1 in TGF-β1- versus vehicle-treated cells (FIG. 12F). Notably, TGF-β1 stimulation decreased expression of TET2 and TET1 (FIG. 12F), consistent with observations from human fibrotic livers showing increased expression of TET3 and decreased expression of TET2 and TET1 (FIG. 12G). Strikingly, when the same set of human liver tissue samples were analyzed, the results (FIG. 12G) essentially mirrored those seen in the primary human HSCs (FIG. 12F). Furthermore, immunohistochemistry (IHC) analysis of human fibrotic liver tissues revealed strong colocalizations of TGF-β1 and TET3 proteins with activated HSCs (FIG. 12H and FIG. 12I). Based on data from LX-2, primary human HSCs, and human fibrotic liver tissues, the present invention, in part, suggests a signal transduction pathway highlighting the TGF-β1/TET3 double-positive feedback loop in regulation of profibrotic gene expression (FIG. 12J).

Inhibition of TET3 Attenuates Hepatic Fibrosis

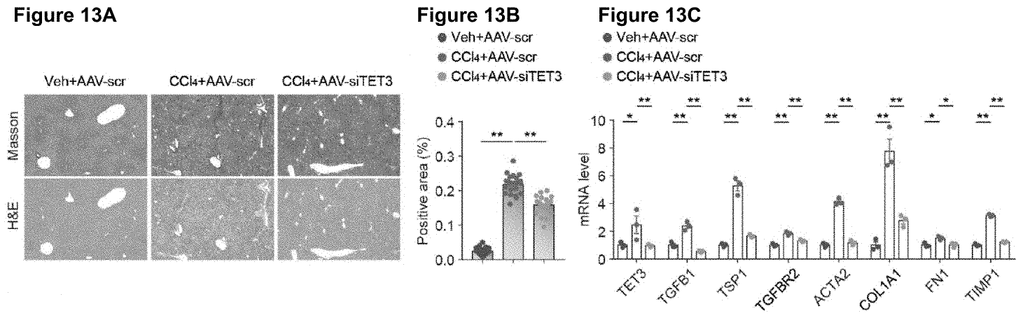

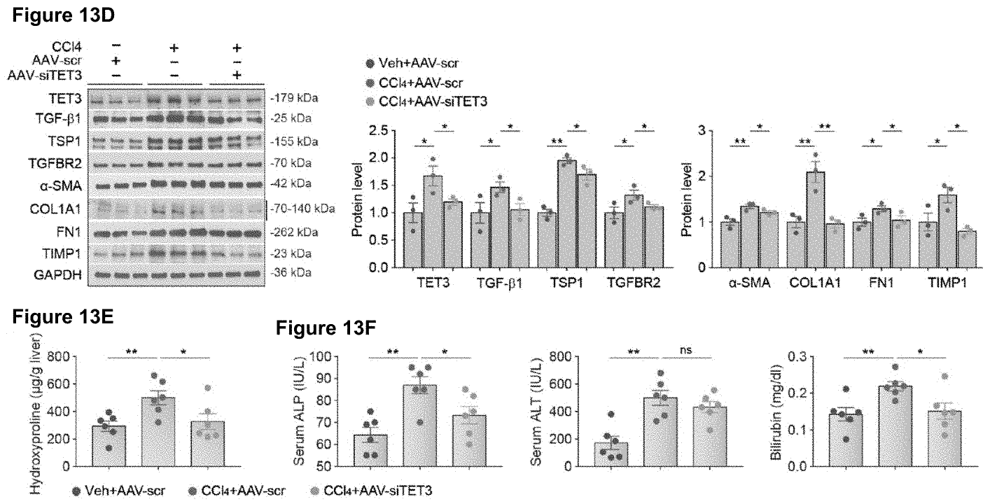

As proof of principle, the pathway disclosed in FIG. 12J was tested using the CCl4 mouse model of liver fibrosis. Mice were administrated with vehicle plus AAV-scr (group 1), CCl4 plus AAV-scr (group 2), or CCl4 plus AAV-siTET3 (group 3). The AAV-siTET3 virus expresses siRNAs targeting mouse TET3 from an adeno-associated virus (serotype 8)-based vector. The AAV-scr virus expresses non-targeting scrambled siRNAs. Vehicle (mineral oil) or CCl4 was injected i.p. twice a week; AAV-scr or AAV-siTET3 was injected via tail vein once a week. Five weeks following the initial injection, mice were sacrificed and blood and tissue samples were collected. As shown in FIG. 13A and FIG. 13B, group 1 mice did not develop liver fibrosis, group 2 mice developed liver fibrosis, and group 3 mice developed liver fibrosis but significantly less than group 2 mice. Gene expression analysis revealed significantly increased expression of key pathway genes TET3, TGFB1, TSP1, TGFBR2, ACTA2, COL1A1, FN1, and TIMP1 in liver tissues from group 2 mice as compared to group 1 mice at levels of both mRNA and protein (FIG. 13C and FIG. 13D). The increase in expression of key pathway genes was significantly attenuated in liver tissues from group 3 mice as compared to group 2 mice (FIG. 13C and FIG. 13D). Liver tissue hydroxyproline, which is a unique amino acid in collagen molecules and an important biomarker of liver fibrosis, was also examined (Lee H S et al., 2005, J Gastroenterol Hepatol 20:1109-1114). The hydroxyproline content was significantly increased in group 2 mice as compared to group 1 mice, but the increase was abolished in group 3 mice (FIG. 13E). Blood chemistry results showed elevated alkaline phosphatase (ALP), alanine transaminase (ALT), and bilirubin in group 2 mice as compared to group 1 mice, suggesting impaired liver function (FIG. 13F). However, these markers were reduced in group 3 mice (FIG. 13F). Taken together, these data provide strong in vivo evidence supporting TET3 as a novel regulator of TGF-β signaling and suggest that the TET3/TGF-β double-positive feedback loop may be a promising therapeutic target for liver fibrosis.

Pathological increase in TGF-β signaling is a principal driver of fibrosis in multiple organs including liver, lung, kidney, skin, heart, and uterus (Meng X M et al., 2016, Nat Rev Nephrol 12:325-338; Murphy-Ullrich J E et al., 2018, Matrix Biol 68-69:28-43; Chegini N, 2010, Semin Reprod Med 28:180-203). Multiple strategies aimed at inhibiting individual TGF-β components including TGF-β, its receptors and activators have been proposed and tested in preclinical studies. For the first time, TET3 was identified as a potent activator and also a downstream effector of TGF-β signaling using liver fibrosis as a model. It was also shown that TET3 activates TGF-β signaling by simultaneously increasing expression of multiple key TGF-β pathway genes at multiple levels (the ligand, the receptor and the activator) via an epigenetic mechanism. As such, the present invention, in part, highlights the clinical significance of this newly identified TET3/TGF-β1 double-positive feedback mechanism in human fibrotic liver tissues and in a mouse model of liver fibrosis.

Although not bound by any particular theory, it is expected that TET3 regulates more genes than just the three (TGFB1, TSP1, and TGFBR2) tested here and that TGF-β signaling is only among the many pathways that TET3 may impact to regulate fibrosis. The molecular and functional interaction between TET3 and the TGF-β signaling pathway characterized in this invention represents a novel and critical mechanism in liver fibrosis. Curiously, a consistent decrease in expression of both TET1 and TET2 in TGF-β1 stimulated primary human HSCs as well as in human fibrotic liver tissues was observed (FIG. 13F and FIG. 13G). This is consistent with a recent study showing decreased TET2 expression in human fibrotic livers with alcoholic liver disease (Page A et al., 2016, J Hepatol 64:661-673). However, the same study reported unaltered TET3 expression (Page A et al., 2016, J Hepatol 64:661-673). Although not bound by any particular theory, the apparent discrepancy on TET3 expression in fibrotic human livers could be due to different underlying liver diseases (alcoholic liver disease versus viral liver disease). Importantly, the present invention also describes the first demonstration of a clear mechanistic connection between TET3, TGF-β signaling, and hepatic fibrosis.

Recently, humanized monoclonal antibodies neutralizing TGF-β isoforms have been tested in human patients with systemic sclerosis and kidney diseases. These short-term and small-scale clinical trials have been associated with severe side effects. This likely was at least in part due to the pleiotropic roles of TGF-β in broad physiological/biological processes. As TET3 is pathologically induced by TGF-β1 during liver fibrosis (this study), targeting TET3 may produce more localized and specific effects. Given that TET3 is an enzyme, it is likely more druggable than TGF-β. Finally, as aberrant TGF-β activation is a universal mechanism of fibrosis, the discovery of the TET3/TGF-β double-positive feedback mechanism may have broad and far-reaching implications in fibrotic disease control.

### Example 3: H19 lncRNA Regulates Uterine Leiomyomas Driver Genes

H19 Expression is Aberrantly Elevated in Fibroids

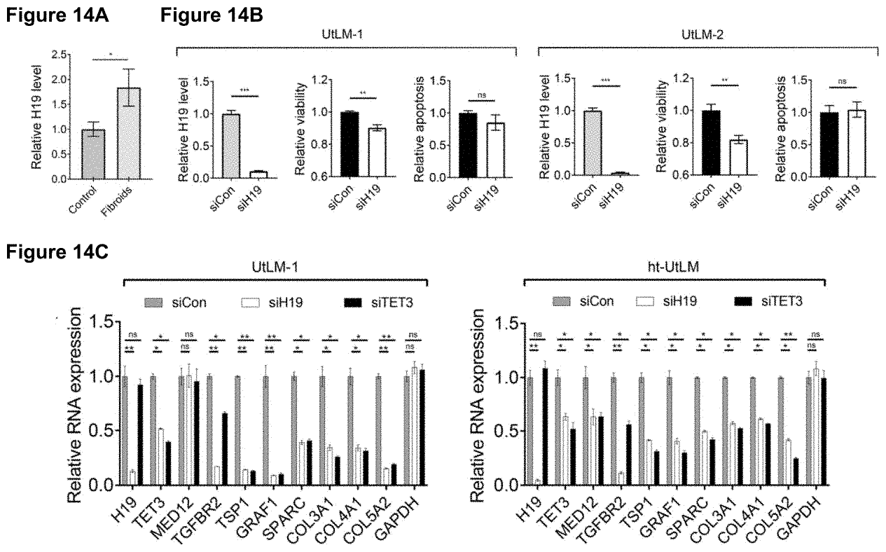

Uterine fibroids are a fibrotic disorder characterized by excessive ECM deposition (Stewart E A et al., 2016, Nat Rev Dis Primers 2:16043; Chegini N et al., 2010, Semin Reprod Med 28:180-203). As H19 has been implicated in the fibrosis of liver, lung and kidney (Song Y et al., 2017, Hepatology 66:1183-1196; Lu Q et al., 2018, Inflammation 2018; Xie H et al., 2016, Oncotarget 7:51473-51481), tests, described herein, were performed to assess whether H19 expression is altered in uterine fibroids. Thus, paired fibroids (Fibroids) and myometrial tissues (Control) were collected from women at the proliferative stage of the menstrual cycle who underwent a hysterectomy or myomectomy for fibroids. RNAs were extracted from the tissue samples and H19 expression levels were determined by reverse transcription and quantitative real-time PCR (RT-qPCR) analysis. A statistically significant increase in H19 expression in fibroids versus matched myometrium was observed (FIG. 14A), suggesting a role of H19 in uterine fibroids.

H19 Promotes Proliferation of Leiomyoma Cells

To assess the role of H19 in cell growth, H19 knockdown experiments were performed on primary human leiomyoma cells (UtLM-1 and UtLM-2) derived from the fibroid tumors of two patients. Thus, H19-specific siRNA (siH19; Ghazal S et al., 2015, EMBO Mol Med 7:996-1003) or control siRNA (siCon) were transfected into cells, followed by analyses of cell viability (as a readout for cell proliferation) and caspase 3/7 activity (as a readout for cell apoptosis) at 48 h posttransfection. When H19 was downregulated (FIG. 14B, left panels), cell viability decreased (middle panels) without affecting apoptosis (right panels), suggesting that H19 positively affects leiomyoma cell proliferation.

H19 Regulates Expression of Fibroid-Promoting Genes

To begin to elucidate the mechanism of H19-mediated regulation, H19 knockdown studies were carried out in normal human primary uterine smooth muscle cells (UtSMC). RNAs were extracted at 48 h post-transfection and subjected to high throughput deep sequencing (RNA-seq). Results showed that among the numerous genes whose expressions were downregulated in siH19-transfected cells were those involved in 5hmC epigenetic regulation, TGF-β signaling, ECM remodeling, and cell growth (GEO accession number GSE110557). Decreased expression of genes (TET3, MED12, TGFBR2, TSP1, GRAF1, SPARC, COL3A1, COL4A1 and COL5A2) previously implicated in fibroids was also observed following H19 knockdown in primary human leiomyoma cells (FIG. 14C, left panel, compare white bars to gray bars across all columns) as well as in ht-UtLM, an immortalized human leiomyoma cell line (Carney S A et al., 2002, Lab Invest 82:719-728) (FIG. 14C, right panel). Remarkably, when TET3 was downregulated using siRNA (siTET3) (FIG. 14C, second columns from left, compare black bars to gray bars), results similar to those of H19 knockdown were obtained (FIG. 14C, compare black bars to gray bars across all columns). The lack of effects of H19 or TET3 knockdown on MED12 expression in ht-UtLM cells was likely due to a much lower endogenous level of H19 as compared to UtLM cells. To determine whether decreased mRNA expression leads to decreased protein levels, Western blot analysis was conducted using UtLM cells. Results showed that when H19 (FIG. 14C, first column from left, compare white bar to gray bar) or TET3 (FIG. 14C, second column from left, compare black bar to gray bar) was downregulated, the protein levels of MED12, TGFBR2, TSP1, GRAF1 and SPARC, COL5A2, COL4A1, and COL3A1 all decreased, consistent with mRNA results (FIG. 14C). Collectively, these results suggested that H19 positively regulates expression of a subset of fibroid promoting genes including TET3, which appears to be also an important downstream mediator of H19.

H19 Regulates HMGA2 and TET3 Expression Via the H19/Let-7 Axis

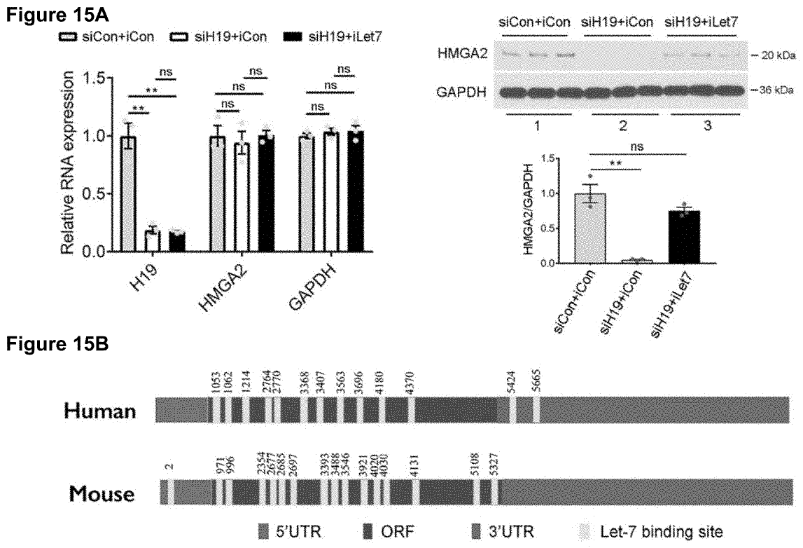

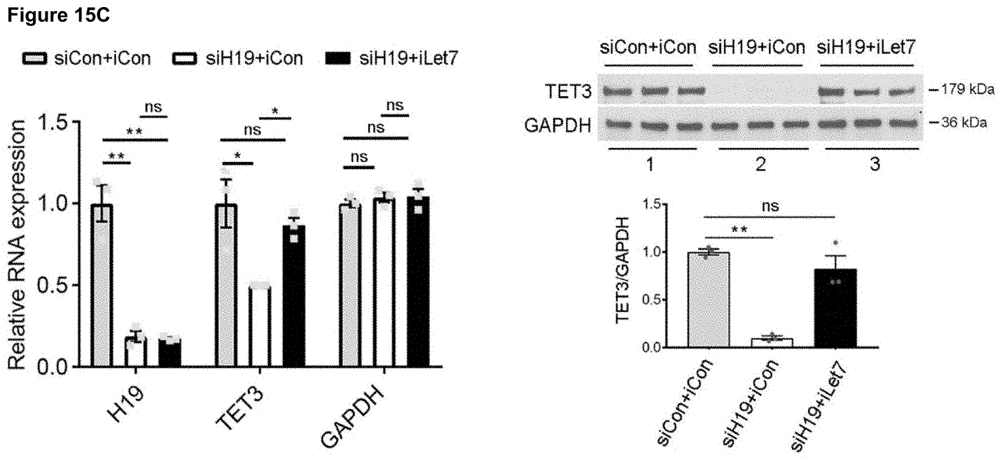

Previous work documented the H19/let-7 axis where H19 acts as a “molecular sponge” to reduce the bioavailability of let-7: H19 contains multiple let-7-binding sites that sequester let-7 and prevent it from binding to target mRNAs (Kallen A N et al., 2013, Mol Cell 52:101-112). Binding of let-7 to complementary sequences in target mRNAs results in translational repression and/or mRNA degradation. Therefore, let-7 action can lead to decreased protein levels with or without altering mRNA levels (Fabian M R et al., 2012, Nat Struct Mol Biol 19:586-593). HMGA2 contains let-7-binding sites in its 3′UTR and is a validated target of let-7 (Lee Y S et al., 2007, Genes Dev 21:1025-1030; Mayr C et al., 2007, Science 315:1576-1579). In the previous studies of human endometrial cancer and ovarian cancer cells, H19 was shown to positively regulate HMGA2 expression via the H19/let-7 axis (Yan L et al., 2015, Oncogene 34:3076-3084). As HMGA2 is among the key driver genes in leiomyomas (Mehine M et al., 2016, Proc Natl Acad Sci USA 113:1315-1320), tests were performed to assess whether H19 regulates its expression in leiomyoma cells. Thus, H19 knockdown experiments in combination with a let-7-specific inhibitor (iLet7) (Ghazal S et al., 2015, EMBO Mol Med 7:996-1003; Zuckerwise L et al., 2016, Oncotarget 7:38398-38407) were carried out in UtLM cells, followed by analysis of HMGA2 expression. iLet7 are chemically modified, single-stranded nucleic acids that bind to let-7 specifically and block its activity. The effect of H19 knockdown (i.e., downregulation of HMGA2) would be abolished in the presence of iLet7 which acts to neutralize let-7 released from H19 sequestration. Indeed, when H19 was knocked down in the absence (FIG. 15A, left panel, left column, compare white bar to gray bar) or presence (compare black bar to gray bar) of iLet7, there was no change in HMGA2 mRNA levels (middle column). However, when H19 was knocked down in absence of iLet7, HMGA2 protein level was significantly decreased (FIG. 15A, right panel, top blots, compare lane 2 to lane 1; bottom graph, compare middle bar to left bar), which was restored to the control level when iLet7 was present (top blots, compare lane 3 to lane 1; bottom graph, compare right bar to left bar). This suggested that H19 deficiency led to enhanced let-7 action, enabling it to repress expression of HMGA2 at the translational level without altering HMGA2 mRNA levels. Thus, although not bound by any particular theory, the data demonstrated that in primary leiomyoma cells, H19 promotes HMGA2 expression at the translational level by reducing the bioavailability of let-7.

To determine how H19 regulates TET3 expression, bioinformatics analysis was performed and predicted multiple let-7-binding sites in the coding regions of both human and mouse TET3 mRNAs (FIG. 15B). This suggested that H19 may promote TET3 expression by sequestering let-7. Thus, H19 knockdown experiments in the absence and presence of iLet7 were performed in UtLM cells, followed by analysis of TET3 expression. H19 knockdown led to decreased TET3 expression at both mRNA (FIG. 15C, left panel) and protein (right panel) levels, but the expression was restored to control levels in the presence of iLet7. These results suggested that TET3 is a novel target of let-7 and that H19 regulates TET3 expression via the H19/let-7 axis.

TET3 Binds to Target Gene Promoters and Regulates DNA Methylation and Histone Modifications

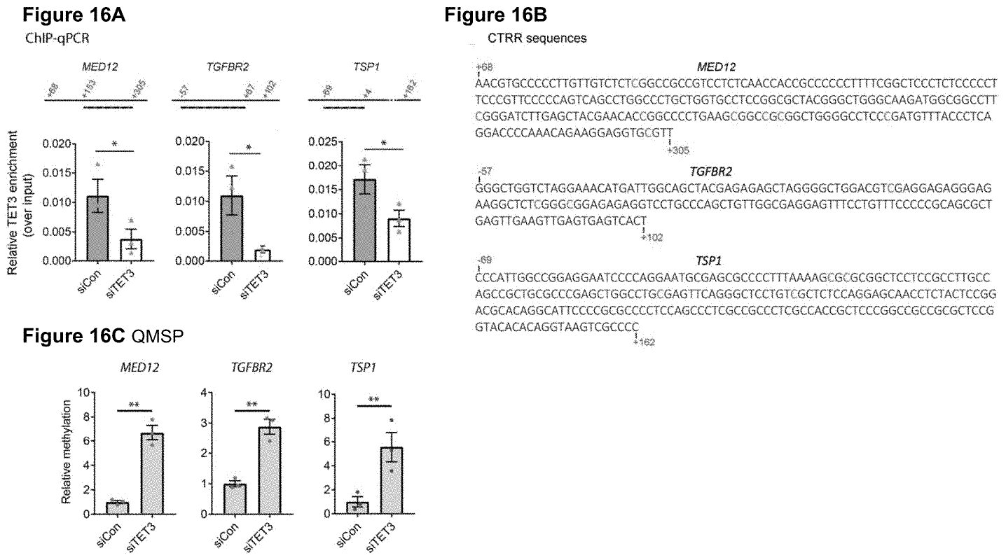

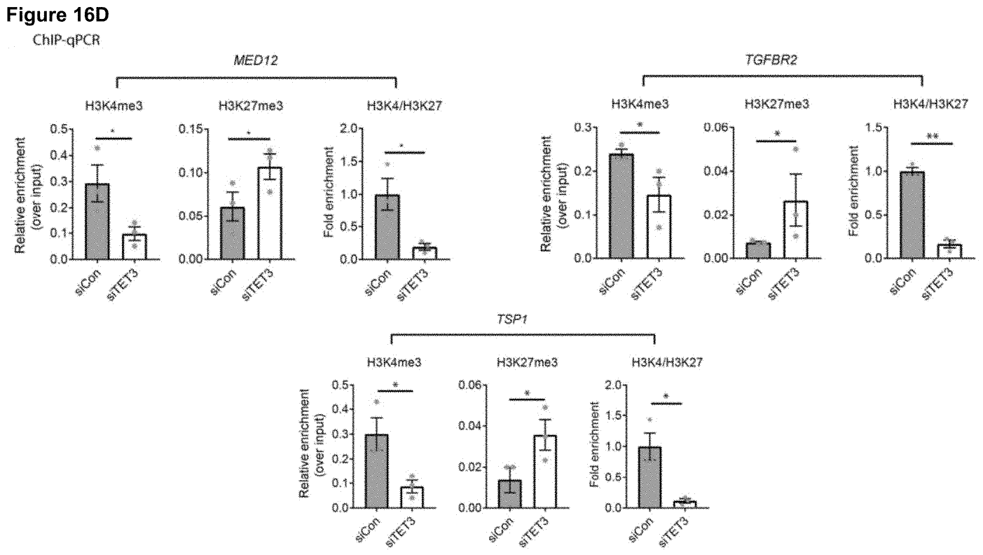

As TET3 positively regulates expression of fibroid-promoting genes, such as MED12, TGFBR2, TSP1, etc. (FIG. 14C), tests were performed to assess the possibility of a direct interaction between TET3 and target genes. Thus, chromatin immunoprecipitation coupled with qPCR (ChIP-qPCR) experiments were performed using a TET3-specific antibody to immunoprecipitate protein-DNA complexes from UtLM cells transfected with siCon or siTET3 for 48 h and qPCR amplified the critical transcriptional regulatory regions (CTRR) of the MED12 (Philibert R A et al., 1999, Hum Genet 105:174-178), TGFBR2 (Yamashita S et al., 2008, Cancer Res 68:2112-2121), and TSP1 (Stenina-Adognravi 0, 2014, Matrix Biol 37:69-82) promoters. In TET3 knockdown cells, binding of TET3 to the respective promoters was significantly reduced as compared to control cells, consistent with physical interactions of TET3 with these promoters (FIG. 16A).

It is well established that TET proteins promote DNA demethylation leading to alteration of chromatin states. First, TET3-induced DNA methylation changes were assessed using ht-UtLM cells. Cells were transfected with siTET3 (or siCon as a control), and genomic DNA was extracted 48 h later and subjected to single-nucleotide resolution genome-wide DNA methylation profiling. As expected, following TET3 knockdown, extensive DNA methylation changes relative to siCon-treated cells were observed, with some genes showing increased methylation, others showing decreased methylation, and a third group with no significant change (GEO accession number GSE117190). Remarkably, increased methylation of the CpGs within the CTRRs of the MED12, TGFBR2 and TSP1 promoters was observed (FIG. 16B, differentially methylated cytosine residues highlighted in red). This was further confirmed in UtLM cells using quantitative methylation-specific PCR (QMSP) analysis (FIG. 16C), the previously established method (Zhou J et al., 2015, Nat Commun 6:10221; Zhong T et al., 2016, Oncogene 36:2345-2354). The QMSP primers were designed based on the differentially methylated cytosine residues (FIG. 16B). Collectively, these results suggested that binding of TET3 to MED12, TGFBR2 and TSP1 induces promoter demethylation.

Next, tests were performed to assess whether TET3 knockdown affects chromatin states. UtLM cells were transfected with siCon or siTET3, followed by ChIP-qPCR, immunoprecipitating with antibodies specific for the H3K4me3 (active) or H3K27me3 (inactive) marks and amplifying the CTRRs of MED12, TGFBR2 and TSP1. ChIP analysis showed that TET3 knockdown significantly decreased H3K4me3 (FIG. 16D, top left columns) and increased H3K27me3 (middle columns) association with all three promoters, such that the ratios of H3K4me3/H3K27me3 decreased by 5-8 fold (right columns). These results suggested that TET3 knockdown promotes a heterochromatin conformation and diminishes chromatin accessibility at the MED12, TGFBR2 and TSP promoter regions.

H19 and TET3 Expression Positively Correlates with Expression of Key Fibroid-Promoting Genes In Vivo

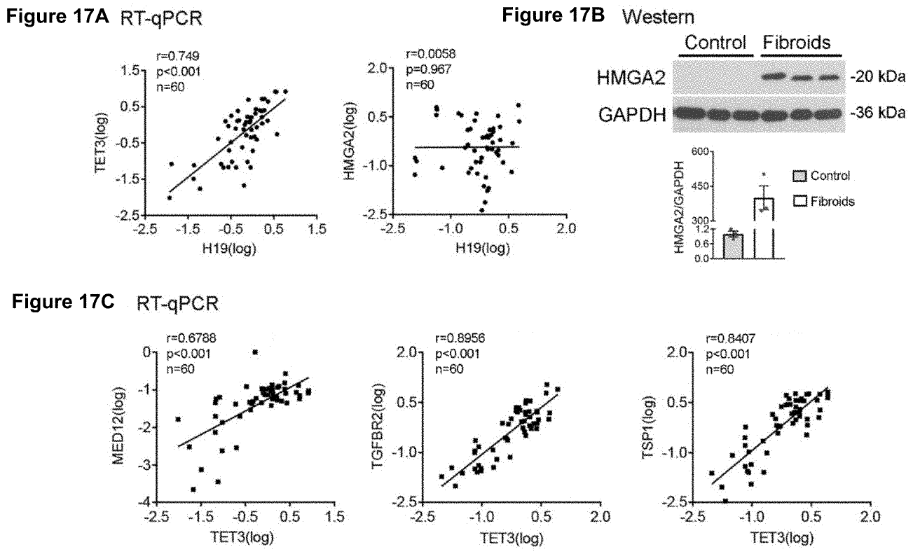

To provide evidence supporting the in vivo roles of H19 and TET3 in fibroids, RT-qPCR analysis was performed on RNA samples derived from fibroids and matched myometrium. There was a positive correlation in expression between H19 and TET3, and the trend was statistically significant (FIG. 17A, left panel), consistent with the in vitro data showing that H19 positively regulates TET3 expression (FIG. 15C). No correlation between H19 and HMGA2 at the RNA level was detected (FIG. 17A, right panel). However, Western blot analysis showed a clear increase at the HMGA2 protein level in fibroids versus normal myometrium (FIG. 17B), consistent with the in vitro observation that H19 promotes HMGA2 expression at the translational level (FIG. 15A). Next, expression of TET3 and its target genes MED12, TGFBR2, and TSP1 in fibroids and matched myometrium was analyzed. As seen in FIG. 17C, TET3 expression was positively correlated with expression of all three target genes. These results corroborated the in vitro findings that TET3 knockdown in primary leiomyoma cells decreased expression of MED12, TGFBR2, and TSP1 (FIG. 14C, left panel) and that TET3 regulates their expression at the epigenetic level (FIG. 16). Taken together, the results strongly support the in vivo roles of H19 and TET3 in regulation of expression of key fibroid-promoting genes.

Steroid Hormones Upregulate Fibroid-Promoting Genes in a H19-Dependent Manner

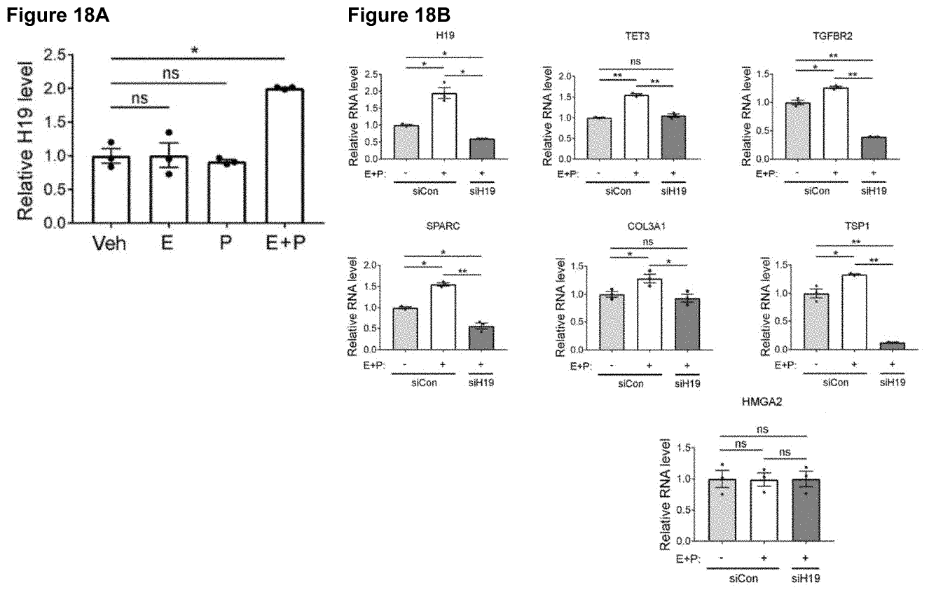

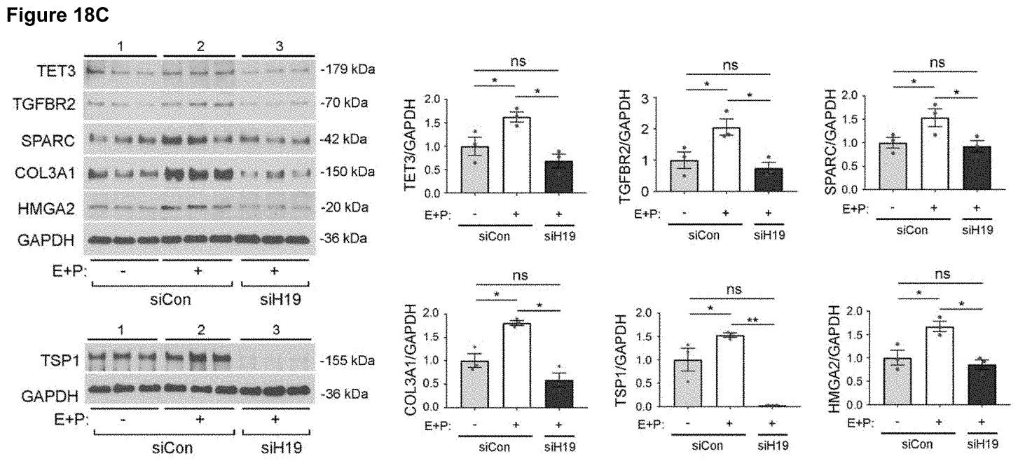

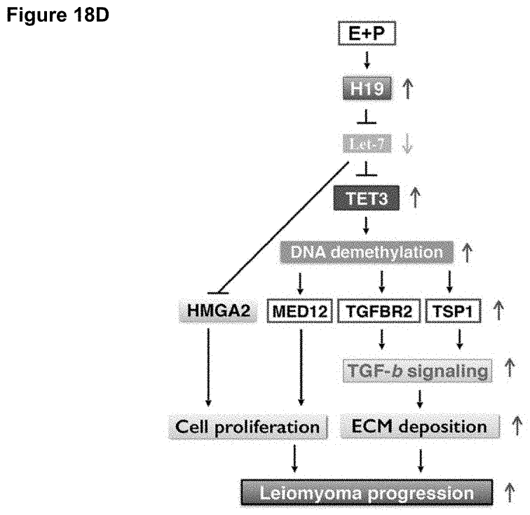

ht-UtLM cells which express a low level of endogenous H19 were treated with estradiol (E), progesterone (P), or E+P, and assessed H19 expression 24 h later. While exposing the cells to E or P alone did not affect H19 expression, a combined treatment with both E and P significantly upregulated H19 (FIG. 18A). To determine whether E+P regulate fibroid-promoting genes and whether the effects are influenced by H19, ht-UtLM cells were treated with E+P in combination with H19 knockdown and a subset of H19-regulated genes were tested. E+P increased the expression of H19, TET3, TGFBR2, SPARC, COL3A1, and TSP1 both at the levels of mRNA (FIG. 18B, compare middle bars to left bars) and protein (FIG. 18C, left panels, compare lanes 2 to lanes 1; right panels, compare middle bars to left bars), and this effect was abolished when H19 was downregulated (FIG. 18B, compare right bars to middle and left bars; C, left panels, compare lanes 3 to lanes 2 and 1; right panels, compare right bars to middle and left bars). While E+P and H19 knockdown did not affect HMGA2 at the mRNA level (FIG. 18B), they did at the protein level (FIG. 18C), further supporting the notion that H19 regulates HMGA2 expression at the translational level (FIG. 15A). The fact that H19 knockdown to below basal level (FIG. 18B, compare right bar to left bar) abrogated both E+P- and basal H19-induced effects (compare right bars to left bars; C, left panels, compare lane 3 to lane 1; right panels, compare right bars to left bars) strongly suggests that the E+P-induced expression of fibroid-promoting genes is H19-dependent.

UFs are highly heterogeneous in terms of symptoms, histopathology, treatment requirements, and clinical outcomes. Emerging evidence suggests that genetic alterations and subtype-specific gene expression changes underlie the pathogenesis and heterogeneity of UFs. MED12 is mutated in 70% of fibroids (Makinen N et al., 2011, Science 334:252-255) and a gain-of-function mutation of MED12 induces fibroid formation and genomic instability in mice (Mittal P et al., 2015, J Clin Invest 125:3280-3284). Furthermore, overexpression of wild-type MED12 promotes proliferation of leiomyoma cells (Al-Hendy A et al., 2017, Endocrinology 158:592-603). Together, these findings strongly point to a causal role of MED12 in fibroids. Another gene that has garnered much attention is HMGA2, a well-studied oncogene (Lee Y S et al., 2007, Genes Dev 21:1025-1030), due to its frequent rearrangements and overexpression in fibroids (Mehine M et al., 2014, Fertil Steril 102:621-629). Notably, genome-wide analyses of fibroids harboring different genetic alterations have revealed fibroid subtypes with distinct driver pathway genes, and MED12 and HMGA2 have been found to be the most common driver genes that together account for nearly 90% of all fibroids (Mehine M et al., 2016, Proc Natl Acad Sci USA 113:1315-1320; Mehine Metal., 2014, Fertil Steril 102:621-629). Remarkably, the results demonstrated that H19 acts as an upstream regulator of both MED12 and HMGA2. Furthermore, H19 was shown to regulate their expression via distinct mechanisms: it promotes HMGA2 expression post-transcriptionally by reducing the bioavailability of let-7 (FIG. 15), whereas enhancing MED12 expression epigenetically via the action of the DNA demethylase TET3, which was identified as a novel target of let-7-mediated regulation (FIG. 18D).

The most striking characteristic of UFs is the deposition of excessive amounts of ECM, whose crucial role in the pathogenesis of UFs has increasingly been recognized. Excessive ECM accumulation contributes significantly to the growth and stiffness of fibroid tumors. In addition to providing structure to the fibroid, the ECM actively participates in various biochemical signaling processes responsible for cell adhesion, proliferation, survival, differentiation and migration. The ECM also serves as a reservoir for growth hormones and soluble profibrotic factors that promote tumor growth. Thus, ECM is a potential therapeutic target for UFs (Islam M S et al., 2018, Hum Reprod Update 24:59-85). Many signal transduction pathways affect ECM production and remodeling, with TGF-β being the key driver (Meng X M et al., 2016, Nat Rev Nephrol 12:325-338; Murphy-Ullrich J E et al., 2018, Matrix Biol 68-69:28-43). H19 was shown to promote TGF-β signaling by upregulating expression of TGFBR2 and TSP1 via the TET3-mediated epigenetic mechanism (FIG. 18D). The decreased expression of ECM component genes GRAF1, SPARC, COL3A1, COL4A1, and COL5A2 in addition to TGF-β pathway genes TGFBR2 and TSP1 (FIG. 14) following H19 or TET3 knockdown further supports the notion that H19 acts through TET3 to promote TGF-β signaling and ECM production (FIG. 18D). The critical role of altered H19 expression, TGF-β signaling, and ECM deposition in the pathogenesis of UFs is further underscored by findings from genome-wide association studies (GWAS) revealing SNPs in H19, TGFBR2, and GRAF1 that affect both the risk and tumor size of UFs (Aissani B et al., 2015, Fertil Steril 103:528-534 e513; Aissani B et al., 2015, Int J Mol Epidemiol Genet 6:9-19).

Despite the prevalence and enormous medical and economic impact of UFs, there are few options for the treatment of UFs and their associated symptoms. Hysterectomy, which prematurely ends a women's reproductive life, remains a predominant method for treating UFs. The currently available non-surgical interventions include GnRH analogues which cause prolonged suppression of pituitary gonadotropins but are not FDA approved for long-term use and cause significant side effects including menopausal symptoms, and progesterone receptor modulators which temporally relieve symptoms but are not available in the United States and have been associated with liver failure in Europe (Stewart E A et al. 2016, Nat Rev Dis Primers 2:16043; Styer A K et al., 2016, Best Pract Res Clin Obstet Gynaecol 34:3-12). The existence of multiple fibroid subtypes driven by distinct pathway genes has further complicated treatment decisions and effectiveness. Herein described identification of H19 as a master regulator of key driver genes including MED12, HMGA2, and ECM remodeling genes (FIG. 18D) suggests a potentially unifying mechanism mediated by H19. Finally, progesterone in combination with estradiol (but not alone) was shown to induce fibroid-promoting gene expression in a H19-dependent manner (FIG. 18). This is intriguing because in a leiomyoma xenograft mouse model, a combined treatment of estradiol and progesterone induced tumor growth but estradiol alone did not (Ishikawa H et al., 2010, Endocrinology 151:2433-2442). The same study also showed that estradiol induced expression of progesterone receptors in leiomyoma cells (Ishikawa H et al., 2010, Endocrinology 151:2433-2442). It will be important and fascinating to test in the future whether targeting H19 leads to inhibition of uterine fibroids without inducing a menopause-like state.

In conclusion, this report shows that H19 expression is significantly increased in fibroids as compared to normal myometrium and that H19 functions to promote leiomyoma cell proliferation and expression of MED12, HMGA2, TET3 and ECM remodeling genes. Mechanistically, H19 was demonstrated to regulate gene expression via both posttranscriptional and TET3-dependent epigenetic mechanisms. Taken together with the notion that a single nucleotide polymorphism (SNP) of H19 is linked to increased risk and tumor size of fibroids (Aissani B et al., 2015, Fertil Steril 103:528-534 e513; Aissani B et al., 2015, Int J Mol Epidemiol Genet 6:9-19), the results suggest a critical role of H19 in the pathogenesis of uterine fibroids.

### Example 4: H19/TET1 Axis Promotes TGF-β Signaling Linked to EndMT and Cardiovascular Disease

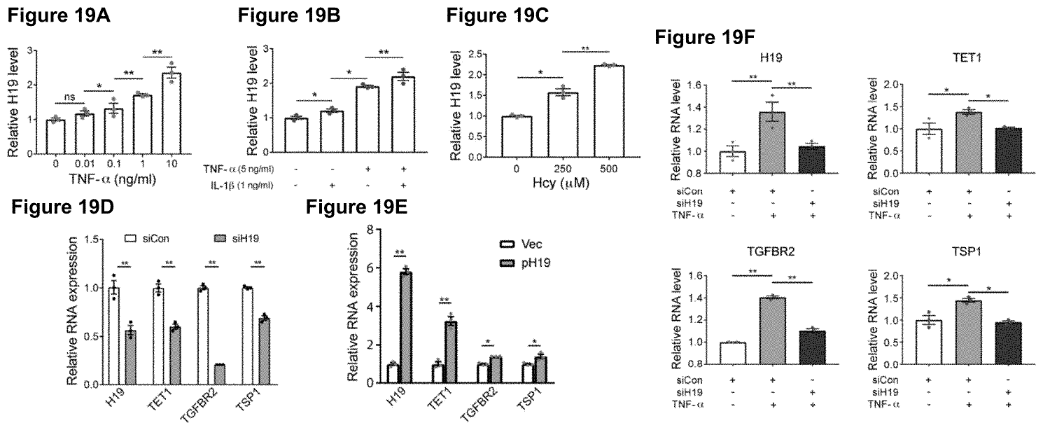

H19 Expression is Induced by Inflammatory Cytokines and Homocysteine in Endothelial Cells H19 has been associated with CVD (Gao et al, 2015, Mutat Res 772:15-22; Greco et al, 2016, J Transl Med 14:183; Han et al, 1996, J Clin Invest 97:1276-1285; Li et al, 2018, Circulation 138:1551-1568) and increased H19 has been detected in plasma from patients with CAD (Zhang Z et al, 2017, Sci Rep 7:7491). Although not bound by any particular theory, it was hypothesized that H19 may play a role in vascular endothelial cells and that altered endothelial H19 expression may contribute to CVD. Inflammation and hyperhomocysteinemia (HHcy, circulating levels of Hcy>15 μM) promote endothelial dysfunction and are well-established risk factors for various CVD (Clarke et al, 1991, N Engl J Med 324:1149-1155; Khambhati et al, 2018, Atherosclerosis 276:1-9; Li et al, 2016, Circulation 134:1752-1765; Li et al, 2018, Nat Commun 9:11; McCully, 1969, Am J Pathol 56:111-128; Toole et al, 2004, JAMA 291:565-575). Treatment of human umbilical vein endothelial cells (HUVECs) with inflammatory cytokines TNF-α and IL-1β induced endothelial inflammation and EndMT (Chen et al, 2015, J Clin Invest 125:4514-4528; Chen et al, 2012, Cell Rep 2:1684-1696). It was found that in HUVECs TNF-α stimulates H19 expression in a dose-dependent manner (FIG. 19A) with an additive effect when combined with IL-1β (FIG. 19B). Likewise, Hcy dose-dependently upregulates H19 (FIG. 19C). These results raised the possibility that altered H19 expression may contribute to endothelial dysfunction.

H19 Regulates Expression of TET1 and TGF-β Pathway Genes

To explore the potential role of H19 in endothelial regulation, H19 siRNA knockdown experiments were performed in HUVECs and the expression of DNA demethylase TET1 and TGF-β pathway genes TGFBR2 and TSP1 was analyzed using RT-qPCR. The rationale for focusing on these genes was that the RNA-seq results from primary human uterine smooth muscle cells showed that these were among the most significantly downregulated genes following H19 knockdown. H19 knockdown in HUVECs reduced expression of all three genes (FIG. 19D). In contrast, H19 overexpression by transfection of a human H19-expression vector pH19 (Deng et al, 2017, Cell Death Dis 8:e3175; Ghazal et al, 2015, EMBO Mol Med 7:996-1003) increased their expression (FIG. 19E). Further, TNF-α upregulated expression of TET1, TGFBR2 and TSP1 (FIG. 19F, compare middle bars to left bars), which was abrogated when H19 was downregulated (FIG. 19F, compare right bars to middle bars). These results suggested that in endothelial cells H19 positively regulates expression of TET1, TGFBR2, and TSP1 and that TNF-α upregulates these genes in a H19-dependent manner.

H19 Regulates TET1 Expression Via the H19/Let-7 Axis

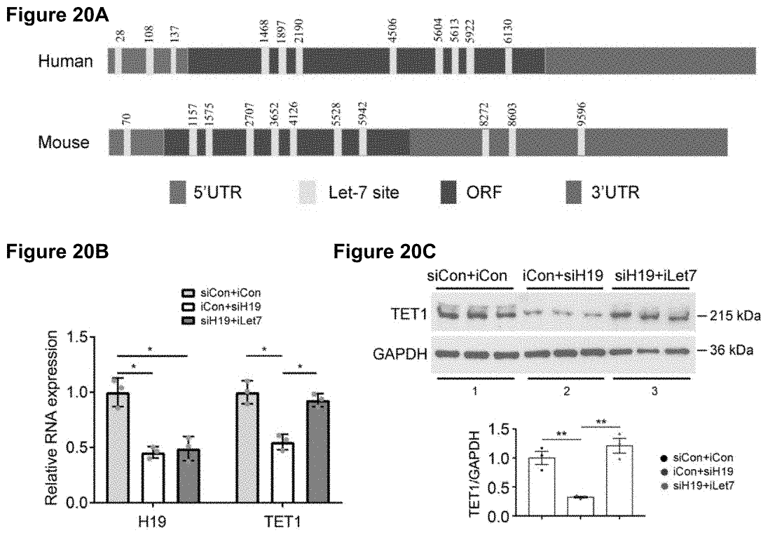

It was previously documented the H19/let-7 axis where H19 acts as a molecular sponge for microRNA let-7, thereby reducing its bioavailability and preventing it from inhibiting target gene expression at the posttranscriptional level (Gao et al, 2014, Nucleic Acids Res 42:13799-13811; Geng et al, 2018, Diabetes 67:2183-2198; Ghazal et al, 2015, EMBO Mol Med 7:996-1003; Kallen A N et al., 2013, Mol Cell 52:101-112; Yan L et al., 2015, Oncogene 34:3076-3084; Zuckerwise L et al., 2016 Oncotarget 7:38398-38407). Binding of let-7 to complementary sequences in target mRNAs results in translational repression and/or mRNA degradation (Fabian et al., 2012, Nat Struct Mol Biol 19:586-593). Thus, it is the relative expression levels of H19 and let-7 (rather than their absolute expression levels) that determine the outcome of let-7 target gene expression. First, analysis was performed to determine how H19 regulates TET1. Bioinformatics analysis was performed and predicted multiple let-7-binding sites in both human and mouse TET1 mRNAs, with the binding sites concentrated in the open reading frame (FIG. 20A). This suggested that H19 may promote TET1 expression by sequestering let-7. To test this possibility, a previously reported methodology to perform H19 knockdown experiments in combination with a let-7-specific inhibitor (iLet7) was used (Gao et al, 2014, Nucleic Acids Res 42:13799-13811; Geng et al, 2018, Diabetes 67:2183-2198; Ghazal et al, 2015, EMBO Mol Med 7:996-1003; Yan L et al., 2015, Oncogene 34:3076-30845; Zuckerwise L et al., 2016 Oncotarget 7:38398-38407) in HUVECs cells. iLet7 are chemically modified, single-stranded nucleic acids that specifically bind to let-7 and block its activity. The effect of H19 knockdown (i.e., downregulation of TET1) was expected to be abrogated in the presence of iLet7 which acts to neutralize let-7 released from H19 sequestration (Gao et al, 2014, Nucleic Acids Res 42:13799-13811; Geng et al, 2018, Diabetes 67:2183-2198; Ghazal et al, 2015, EMBO Mol Med 7:996-1003; Yan L et al., 2015, Oncogene 34:3076-3084; Zuckerwise L et al., 2016 Oncotarget 7:38398-38407). Indeed, H19 knockdown resulted in decreased TET1 expression at both the mRNA (FIG. 20B) and protein (FIG. 20C) levels and the expression was restored to control levels in the presence of iLet7. These results suggested that TET1 is likely a target of let-7 and that H19 regulates TET1 expression at the post-transcriptional level via the H19/let-7 axis.

TET1 Regulates Expression of TGFBR2 and TSP1 at the Epigenetic Level

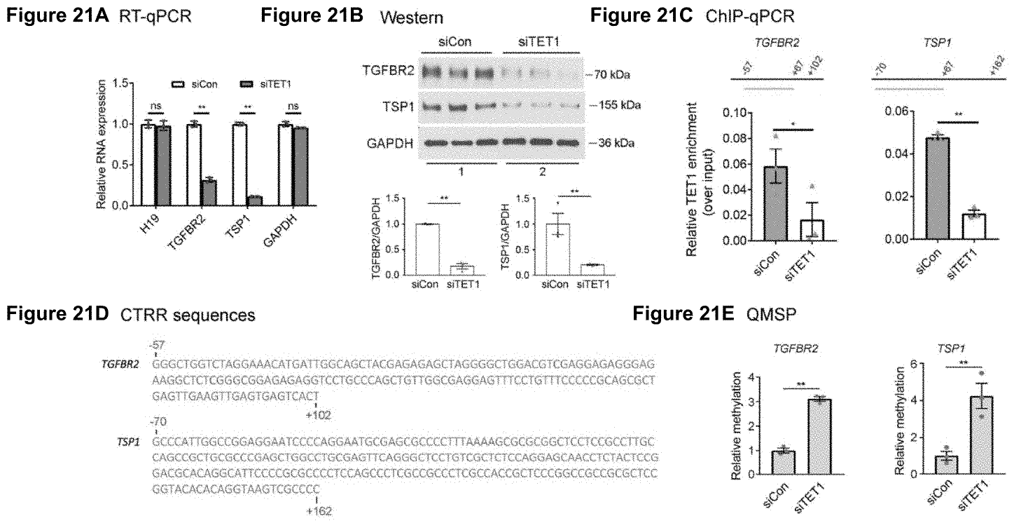

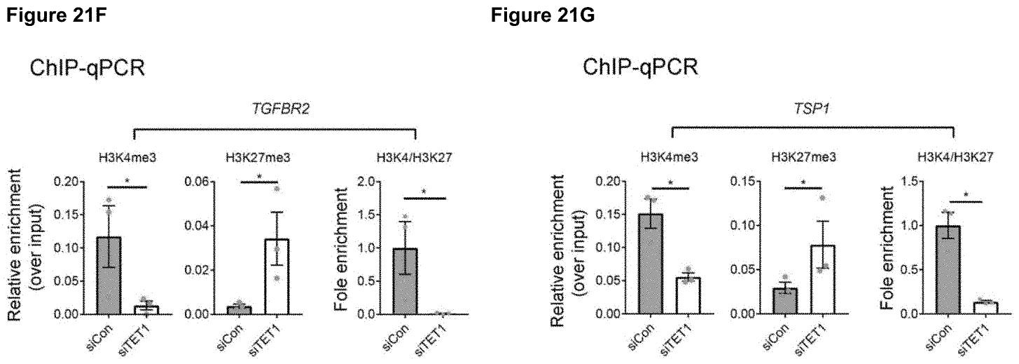

TET2, which normally expresses in mature SMCs, acts to promote key pro-contractile gene expression at the epigenetic level. Mechanistically, TET2 binds to target gene promoters and modifies histones at these promoters, leading to transcription activation (Liu et al, 2013, Circulation 128:2047-2057). In light of these previous findings, further studies focused on whether TET1 regulates gene expression in a similar manner. Thus, knocked down TET1 in HUVECs was generated and decreased expression of TGFBR2 and TSP1 at both mRNA (FIG. 21A) and protein (FIG. 21B) levels was observed without affecting H19 expression, suggesting that TET1 positively regulates expression of TGFBR2 and TSP1. Although TET1 most likely also targets other genes, in the present example, focus was given to TGFBR2 and TSP1 as they are among the most critical upstream genes of TGF-β signaling. Thus, the possibility of a direct interaction between TET1 and these genes was tested. Chromatin immunoprecipitation coupled with qPCR (ChIP-qPCR) experiments were performed using a TET1-specific antibody to immunoprecipitate protein-DNA complexes from HUVECs transfected with siCon or siTET1 and qPCR amplified the critical transcriptional regulatory regions (CTRRs) of the TGFBR2 (Yamashita S et al., 2008, Cancer Res 68:2112-2121) and TSP1 (Stenina-Adognravi 0, 2014, Matrix Biol 37:69-82) promoters. In TET1 knockdown cells, binding of TET1 to the respective promoters was significantly reduced as compared to control cells, consistent with physical interactions of TET1 with the promoters (FIG. 21C).

It is well established that TET proteins promote DNA demethylation leading to alteration of chromatin states. First, tests were performed to assess whether TET1 knockdown alters promoter methylation of TGFBR2 and TSP1. The genome-wide single-nucleotide resolution DNA methylation studies from human uterine cells following TET3 knockdown showed increased methylation in the CTRRs of the TGFBR2 and TSP1 promoters (FIG. 21D). Although not bound by any particular theory, it was hypothesized that TET1 knockdown in HUVECs may induce similar methylation changes in the CTRRs of TGFBR2 and TSP1. Thus, quantitative methylation-specific PCR (QMSP) was performed on DNA isolated from HUVECs using the previously described methods (Zhong T et al., 2016, Oncogene 36:2345-2354; Zhou J et al., 2015, Nat Commun 6:10221). The QMSP primers were designed based on the differentially methylated cytosine residues within the CTRRs (FIG. 21D, red highlighted cytosines). As seen in FIG. 21E, HUVECs treated with siTET1 had an increase in methylation in the CTRRs compared with cells treated with siCon. These results suggested that binding of TET1 to TGFBR2 and TSP1 induces promoter demethylation in HUVECs.

Next, tests were conducted to assess whether TET1 knockdown affects chromatin states of target genes. HUVECs were transfected with siCon or siTET1, followed by ChIP-qPCR, immunoprecipitating with antibodies specific for the H3K4me3 (active) or H3K27me3 (inactive) marks and amplifying the CTRRs of TGFBR2 and TSP1. ChIP analysis showed that TET1 knockdown significantly decreased H3K4me3 (FIG. 21F and FIG. 21G, left panels) and increased H3K27me3 (middle panels) association with both gene promoters, such that the ratios of H3K4me3/H3K27me3 decreased significantly (right panels). These results suggest that TET1 knockdown likely promotes a heterochromatin conformation, diminishing chromatin accessibility at the TGFBR2 and TSP1 promoter regions.

The H19/TET1 Axis Regulates Expression of TGFBR2 and TSP1

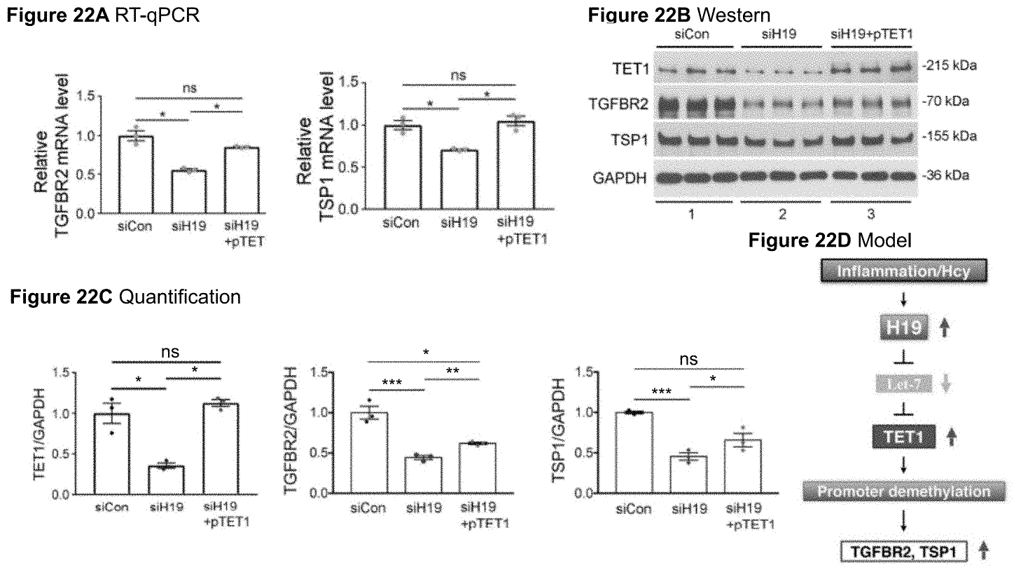

To test whether H19 regulates TGFBR2 and TSP1 via TET1, H19 siRNA knockdown in combination with TET1 overexpression experiments were conducted in HUVECs. Thus, HUVECs were transfected with siCon, siH19 or siH19 together with a human TET1 expression vector pTET1 (Tahiliani et al, 2009, Science 324:930-935). RNA and proteins were analyzed at 48 h and 72 h post-transfection, respectively. H19 knockdown expectedly led to decreased mRNA levels of both TGFBR2 and TSP1 (FIG. 22A, compare middle bars to left bars). However, when H19 was knocked down together with TET1 overexpression, the mRNA levels of TGFBR2 and TSP1 were no longer decreased (FIG. 22A, compare right bars to left bars). The ability of TET1 to rescue the effect of H19 knockdown on TGFBR2 and TSP1 expression was further confirmed at the protein level (FIG. 22B and FIG. 22C). The partial rescue of TGFBR2 by TET1 at the protein level (FIG. 22C, bottom left, compare right bar to left bar) suggested additional translational/posttranslational regulatory mechanisms which warrant future investigation. Based on results shown in FIG. 19 through FIG. 22, a propose pathway was generated as illustrated in FIG. 22D.

H19 and TET1 Upregulate TGF-β Signaling and EndMT Marker Expression In Vitro

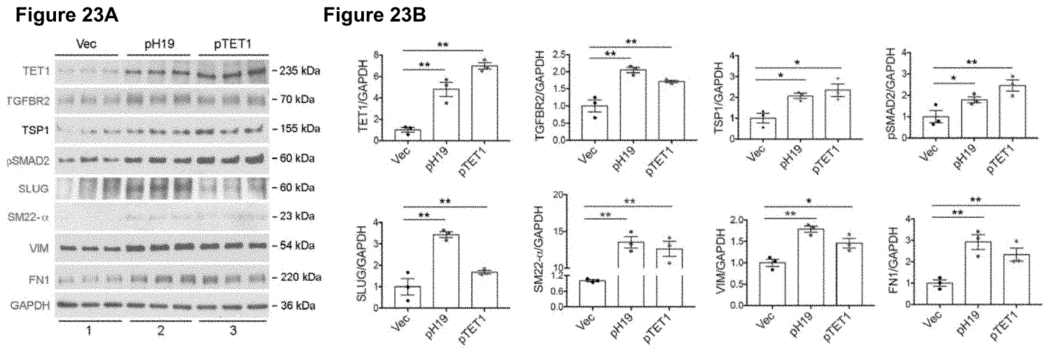

As H19 positively regulates expression of key TGF-β signaling genes TGFBR2 and TSP1 via TET1 (FIG. 22D), further studies were performed to address whether overexpression of H19 or TET1 increases TGF-β signaling and EndMT marker expression. Thus, HUVECs were transfected with pH19 or pTET1 (empty vector as a control) and proteins were extracted 48 h later, followed by Western blot analysis. When H19 or TET1 was overexpressed, the expression of TET1, TGFBR2 and TSP1 was expectedly increased (FIG. 23A, top three blots; FIG. 23B, top three panels on the left). Importantly, there was also an increase in phosphorylation of TGF-β signaling intermediary SMAD2 (p-SMAD2) as well as upregulation of EndMT markers SLUG, smooth muscle 22 alpha (SM22-α), vimentin (VIM) and fibronectin 1 (FN1) (FIG. 23A and FIG. 23B). These results suggested that H19 and TET1 promote TGF-β signaling, contributing to EndMT marker induction in vitro.

H19 Expression Positively Correlates with TGF-β Signaling and EndMT Marker Expression In Vivo

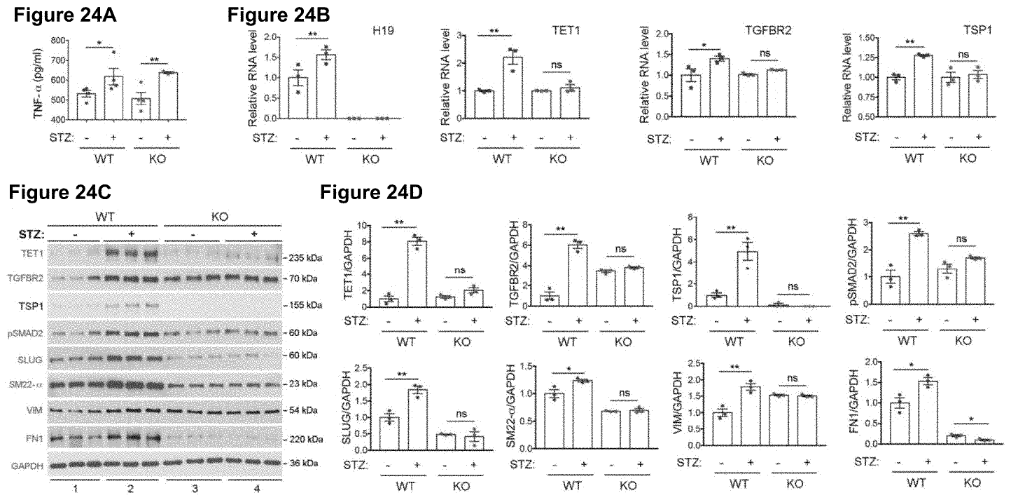

As a first step to address the in vivo significance of H19-mediated activation of TGF-β signaling and possibly EndMT, tests were conducted to assess whether this regulation occurs in the absence of H19 expression using a streptozotocin (STZ)-induced type 1 diabetes mouse model. This model has been used by various groups to study vascular and lung endothelial dysfunction in response to hyperglycemia/oxidative stress-induced inflammation (Li et al, 2016; Popov D et al., 2001, Ital J Anat Embryol 106:405-412; Uyy E et al, 2010, Microvasc Res 79:154-159). Importantly, inflammatory cytokines including TNF-α are known to promote TGF-β signaling and EndMT (Chen et al, 2015, J Clin Invest 125:4514-4528; Chen et al, 2012, Cell Rep 2:1684-1696). Thus, wild-type (WT) and global H19 knockout (KO) (Martinet et al, 2016, Development 143: 962-971; Ripoche et al, 1997, Genes Dev 11:1596-1604; Zhang N et al., 2018, JCI Insight 3:e120304) mice were repeatedly injected with STZ (or vehicle as a control) to induce persistent hyperglycemia (a random plasma glucose level greater than 250 mg/dl that lasted for longer than 2 weeks). At 4 weeks after the initial injection, lung endothelial cells were isolated by affinity purification, followed by gene expression analyses. There was a significant increase in circulating TNF-α levels in STZ- versus vehicle-treated animals in both WT and KO groups (FIG. 24A), suggesting a hyperglycemia/oxidative stress-induced inflammatory milieu. Importantly, H19 expression in ECs from STZ-treated WT animals also increased (FIG. 24B, compare red bar to black bar), consistent with TNF-α-stimulated upregulation of H19 observed in vitro (FIG. 19A). In line with H19-mediated upregulation of TET1 and its downstream target genes, the expression of TET1, TGFBR2 and TSP1 increased in ECs of STZ-treated as compared to vehicle-treated WT animals (FIG. 24C, top three blots, compare lane 2 to lane 1; FIG. 24D, top left three panels). This was accompanied by increased TGF-β signaling (as indicated by increased SMAD2 phosphorylation and SLUG expression) and increased EndMT marker expression (as indicated by increased expression of SM22-α, vimentin, and fibronectin) (FIG. 24B and FIG. 24C). In striking contrast, such changes in expression of TET1, TGF-β signaling genes, and EndMT markers were not observed between STZ- and vehicle-treated groups in the KO animals (FIG. 24C and FIG. 24D). These results strongly suggest an in vivo role of H19 in pathological activation of TGF-β signaling and possibly EndMT.

Endothelial Expression of H19 and TET1 Associates with Human Atherosclerosis

It was previously documented that the extent of CAD in patients strongly correlated with endothelial activation of TGF-β signaling and the extent of EndMT (Chen et al, 2015, J Clin Invest 125:4514-4528). In this previous report immunofluorescence studies on human left main coronary arteries with normal coronaries (no neointima), mild CAD (intima-to-media [I/M] ratio <0.2, grade I plaque), moderate CAD (I/M ratio 0.2-1.0, grade II plaque), or severe CAD (I/M ratio >1.0, grade III and IV plaque) were conducted. Significantly increased expressions of p-SMAD2 (a marker of activated TGF-β signaling) and NOTCH3, SM22-α, collagen 1, and fibronectin 1 (markers of EMT and EndMT) in luminal coronary ECs were detected in CAD, with a strong positive linear relationship between the I/M ratio and number of ECs expressing the markers (Chen et al, 2015, J Clin Invest 125:4514-4528).

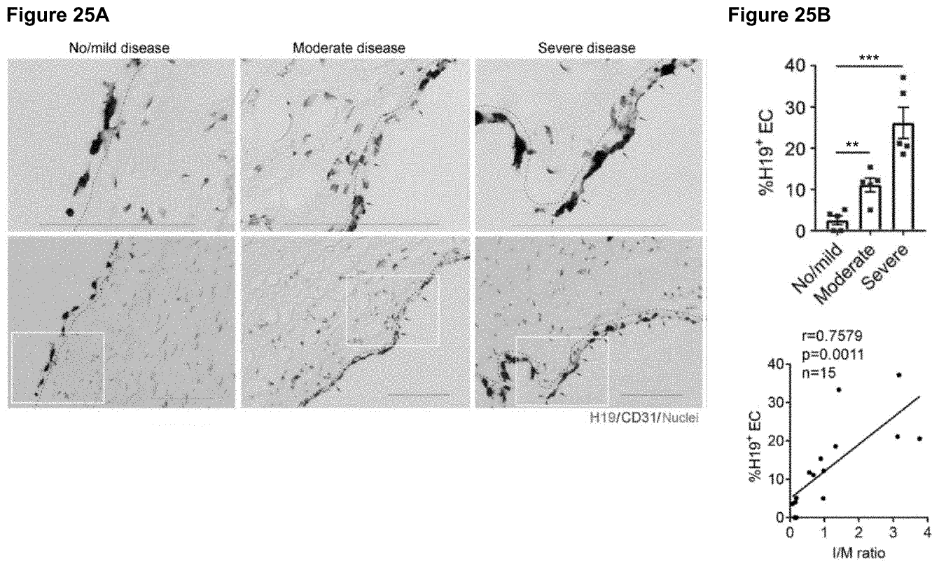

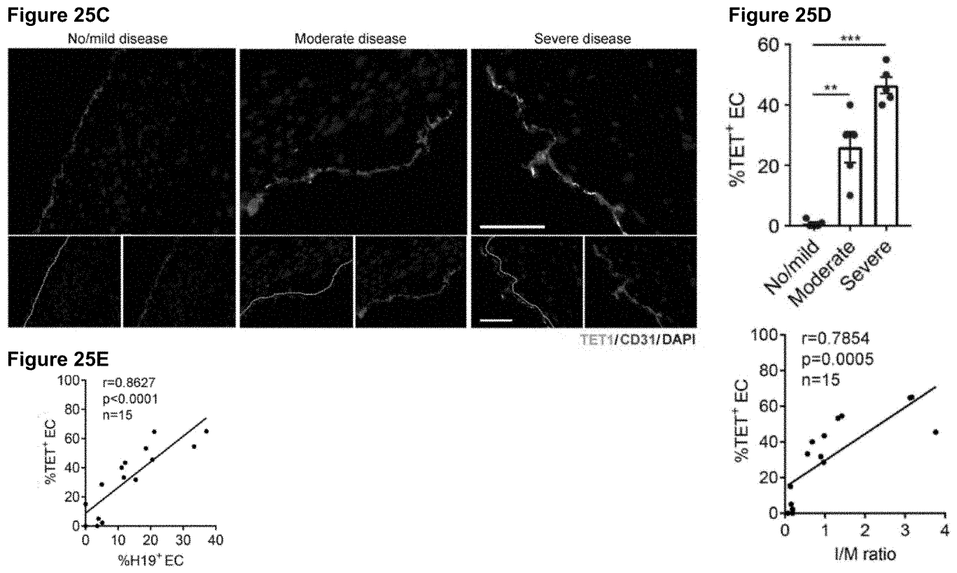

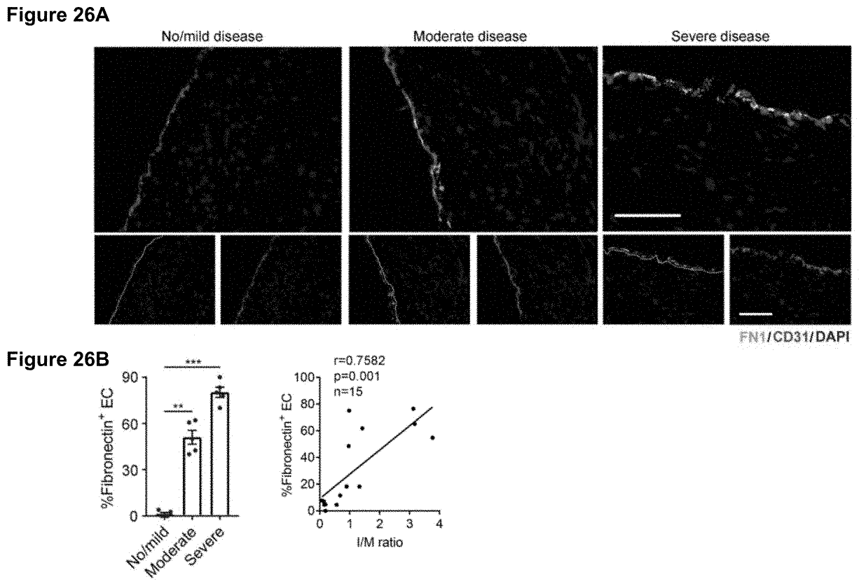

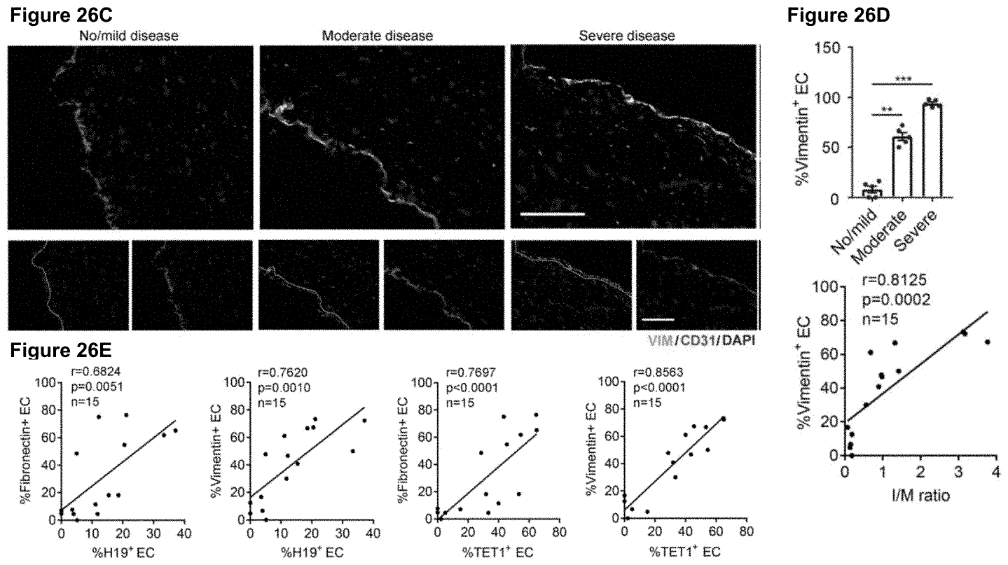

To assess the clinical relevance of H19 and TET1 expression in CAD, RNA in situ hybridization and immunofluorescence studies were performed on left main coronary arteries derived from the same patient cohort (Chen et al, 2015, J Clin Invest 125:4514-4528). While essentially no luminal coronary ECs in patients with no/mild CAD expressed H19, this was increased to 12% of the luminal endothelium in patients with moderate CAD and 26% in patients with severe CAD (FIG. 25A and FIG. 25B). There was a strong positive correlation between the I/M ratio and number of ECs expressing H19 (r=0.75, p=0.001, FIG. 25B). In the same samples, no TET1 expression was detected in patients with no/mild CAD, but TET1 express increased to 25% and 46% of the luminal endothelium in patients with moderate and severe CAD, respectively (FIG. 25C and FIG. 25D). Further, there was a strong linear relationship between the I/M ratio and number of ECs expressing TET1 (r=0.78, p=0.0005, FIG. 25D) as well as expression between H19 and TET1 (r=0.86, p<0.0001, FIG. 25E). Next, the EndMT markers VIM and FN1 were examined in this patient population. Consistent with the previous findings (Chen et al, 2015, J Clin Invest 125:4514-4528), their expression by the luminal ECs strongly correlated with increasing I/M ratio and disease severity (FIG. 26A through FIG. 26D). Moreover, there was a strong positive linear correlation between luminal EC expression of H19 and FN1, H19 and VIM, TET1 and FN1, and TET1 and VIM (FIG. 26E). Taken together with previous findings from the same patient cohort demonstrating a strong association between the extend of coronary atherosclerosis and activation of TGF-β signaling and EndMT (Chen et al, 2015, J Clin Invest 125:4514-4528), these results suggest the endothelial H19/TET1 axis as a possible upstream activator of the TGF-β/EndMT pathway in atherosclerosis.

Pathological activation of TGF-β signaling in endothelial cells promotes EndMT, contributing significantly to CVD (Chen et al, 2015, J Clin Invest 125:4514-4528; Chen et al, 2012, Cell Rep 2:1684-1696; Cooley et al, 2014, Sci Transl Med 6:227ra234; Evrard et al, 2016, Nat Commun 7:11853; Jimenez et al., 2016, Matrix Biol 51:26-36). Yet, the processes leading to endothelial activation of TGF-β signaling and EndMT remain ill-defined. Previous studies have demonstrated that biomechanical and soluble inflammatory stimuli reduce endothelial FGFR1 expression and signaling, leading to increased TGF-β signaling and EndMT (Chen et al, 2012, Cell Rep 2:1684-1696). As such, the present work identifies H19 lncRNA as a novel activator of TGF-β signaling and possibly EndMT. Furthermore, endothelial H19 expression is reported to be induced by inflammatory cytokines and homocysteine in vitro and under diabetic conditions in vivo.

Mechanistically, DNA demethylase TET1 was identified as being a critical downstream effector of H19. It was also demonstrated that TET1 is regulated by let-7 and that H19 enhances TET1 expression post-transcriptionally by reducing the bioavailability of let-7. Herein described results provide evidence that TET1 positively regulates expression of TGFBR2 and TSP1, two key TGF-β signaling pathway genes, at the epigenetic level: it binds to the promoters of TGFBR2 and TSP1, induces DNA demethylation and possibly promotes an euchromatin conformation, thereby increasing gene expression. As such, increased expression of H19 and TET1 was shown to stimulate TGF-β signaling and EndMT marker expression in vitro and that there is a strong correlation between loss of endothelial H19 and lack of activation of TGF-β signaling and EndMT in vivo. Most notably, a striking correlation between increased endothelial expression of H19 and TET1, the extent of EndMT, and severity of atherosclerosis in human coronary arteries was demonstrated. Thus, endothelial activation of the newly identified H19/TET1 axis may play an important role in promoting TGF-β signaling, EndMT, and CVD, although a firm establishment of the link between H19/TET1 and EndMT requires more vigorous investigations including testing with lineage-tracing animal models.

Previous studies have shown that let-7 inhibits expression of TGFBR1 and that decreased endothelial FGFR1 signaling in response to soluble inflammatory stimuli reduces let-7 levels and increases TGFBR1 expression, leading to enhanced TGF-β signaling and EndMT (Chen et al, 2012, Cell Rep 2:1684-1696). In the present study let-7 was shown to inhibit TET1 expression and TET1 was shown to promote expression of TGFBR2 and TSP1 and TGF-β signaling. It was also shown that inflammatory cytokines and homocysteine up-regulate endothelial expression of H19 which acts to reduce the bioavailability of let-7. It remains to be determined whether the FGFR1/TGFBR1 and H19/TET1 pathways operate in a synergistic way in ECs to promote TGF-β signaling and EndMT.

Most studies of TET proteins have focused on their roles in tumorigenesis and the proliferation and differentiation of stem cells. Understanding of the impact of TET proteins on the cardiovascular system is still in its infancy. Recent studies have identified TET2 as a critical modulator of vascular smooth muscle cell phenotype and plasticity (Liu et al, 2013, Circulation 128:2047-2057) as well as having a role in regulation of endothelial cell autophagy (Peng et al, 2016, Oncotarget 7:76423-76436; Yang Q et al, 2016, Ann Biomed Eng 44:2218-2227). The present report is the first to show that TET1 is an important regulator of endothelial cells and possibly EndMT. It is interesting to note that increased TET1 expression is associated with atherosclerosis and severity of the disease (FIG. 25 and FIG. 26) while decreased TET2 expression is associated with atherosclerosis progression (Liu et al, 2013, Circulation 128:2047-2057; Peng et al, 2016, Oncotarget 7:76423-76436; Yang Q et al, 2016, Ann Biomed Eng 44:2218-2227). Future studies will determine whether the apparently opposite effects of TET1 and TET2 are due, in part, to cell type-specific functions (i.e., ECs versus SMCs).

In recent years, many lncRNAs have been identified that play important roles in the cardiovascular system (Uchida S et al., 2015, Circ Res 116:737-750). Only a few studies have characterized the expression and function of lncRNAs in endothelial cells. For example, the hypoxia-inducible, nuclear-localized MALAT1 promotes endothelial cell proliferation and its depletion inhibits vessel growth (Michalik et al, 2014, Circ Res 114:1389-1397). Likewise, the nuclear-localized MANTIS regulates endothelial angiogenic function by modulating the activity of BRG1, the catalytic subunit of a chromatin-remodeling complex (Leisegang et al, 2017, Circulation 136:65-79). Interestingly, a recent paper reported decreased endothelial H19 expression with aging in mice and in human atherosclerotic plaques as compared to healthy arteries (Hofmann et al, 2018, Cardiovasc Res). The authors showed one set of images with localization of H19 by in situ hybridization and CD31 by immunohistochemistry on serial sections (as opposed to co-staining on the same sections) of human carotid artery tissue samples. Whether their observations of H19 expression in human carotid artery samples were actually statistically significant remain to be determined. In the present examples, co-staining of H19 and CD31 was performed by RNA in situ hybridization on the same tissue sections from multiple patients. A statistically significant increase in expression of H19 in the luminal endothelium of coronary atherosclerotic plaques as compared to non-diseased arteries was shown and the expression strongly correlates with the extent of EndMT and severity of the disease (FIG. 25 and FIG. 26). These findings are the first to suggest a link of H19 to endothelial TGF-βsignaling, EndMT, and CVD.

This example shows that H19 expression is induced in ECs by inflammatory cytokines and homocysteine (Hcy), known risk factors for CVD. TET1 expression was also shown to be post-transcriptionally regulated by H19 and that H19 promotes TGF-β signaling and EndMT marker expression via a TET1-dependent epigenetic mechanism. Finally, the H19-mediated regulation is recapitulated in a mouse model of type I diabetes and in human atherosclerosis disease. In summary, a novel regulatory pathway mediated by the H19/TET1 axis that functions to modulate endothelial TGF-β signaling and the likelihood of EndMT that may have a broad implication in cardiovascular disease was identified.

The materials and methods are now described.

### Methods

Materials

Antibodies for TET3 (GeneTex, GTX121453; used at a dilution of 1/500), TGFBR2 (Abcam, ab184948; used at a dilution of 1/1000), TSP1 (Abcam, ab85762; used at a dilution of 1/500), MED12 (Novus Biological, NB100-2357; used at a dilution of 1/500), HMGA2 (Proteintech, 20795-1-AP; used at a dilution of 1/500), GRAF1 (Cell Signaling, 8802; used at a dilution of 1/500), SPARC (Cell Signaling, 8725; used at a dilution of 1/500), COL3A1 (LS-Bio, LS-C159386; used at a dilution of 1/1000), COL4A1 (LS-Bio, LS-C100552; used at a dilution of 1/500), COL5A2 (Origene, TA809611; used at a dilution of 1/500) and GAPDH (Abcam, ab128915; used at a dilution of 1/10000) were purchased. Human H19 siRNA (siH19) and control siRNA (siCon) were previously described (Ghazal S et al., 2015, EMBO Mol Med 7:996-1003). siTET3 (Ambion, 4392420/s47239), estradiol (E8875, Sigma), and progesterone (P0130, Sigma) were purchased. Estradiol and progesterone were dissolved in DMSO and used at a final concentration of 10⁻⁸M.

Antibodies for TET1 (GeneTex, GTX124207; used at a dilution of 1/500 in Western blot and 1:50 in immunofluorescence), TGFBR2 (Abcam, ab184948; used at a dilution of 1/1000 in WB), TSP1 (Abcam, ab85762; used at a dilution of 1/500 in WB), pSMAD2 (Cell Signaling, 3101; used at a dilution of 1/500 in WB), SLUG (Cell Signaling, C19G7; used at a dilution of 1/500 in WB), SM22a (Novus Biological, NBP1-33003; used at a dilution of 1/50 in WB), Vimentin (Abcam, ab92547; used at a dilution of 1/000 in WB and 1:100 in IF), Fibronectin (Abcam, ab194395; used at a dilution of 1/1000 in WB), CD31 (M0823, Dako; used at dilution of 1:100 in IF) and GAPDH (Abcam, ab128915; used at a dilution of 1/10000) were purchased. Human H19 expression vector (pH19), H19 siRNA (siH19), and control siRNA (siCon) were previously described (Ghazal et al, 2015, EMBO Mol Med 7:996-1003). siTET1 (Ambion, 4390771/n368213) were purchased. pTET1 (FH-TET-pEF) (Tahiliani et al, 2009, Science 324:930-935) was a gift from Anjana Rao (Addgene plasmid #49792). Recombinant human TNF-α (R&D Systems, 210-TA), IL-1β (R&D Systems, 201-LB), and L-Homocysteine (Hcy, Sigma, 69453) were purchased. TNF-α and IL-1β were reconstituted in 0.1% bovine serum albumin (BSA)/phosphate-buffered saline (PBS), while Hcy was dissolved in water.

Animals

All animal work was approved by the Yale University Institutional Animal Care and Use Committee. All mice used in this report were male. Mice were housed at 22° C.-24° C. with a 12 h light/12 h dark cycle with normal chow (NC) (Harlan Teklad no. 2018, 18% calories from fat) or high-fat diets (HFD) (Research Diets, D12451, 45% calories from fat) and water provided ad libitum. The H19 KO and WT mice on a background of C57BL/6J have been previously described (Zhang N et al., 2018, JCI Insight 3:e120304; Geng T et al., 2018, Diabetes 67:2183-2198). To induce insulin resistance (FIG. 5C and FIG. 5D), WT C57BL/6J mice were exposed to HFD for 11 days or 12 weeks as previously described (Geng T et al., 2018, Diabetes 67:2183-2198). The diet induced obese and diabetic mice (19-20-week old, fed HFD starting at age of 6-weeks) and Lep^{ob/ob} mice (8-9-week old) used in experiments (FIG. 7F through FIG. 7H, FIG. 8) were purchased from the Jackson Laboratories. Before experiments, mice were allowed to acclimate for at least 7 days in the animal facility.

Animal Model

To induce liver fibrosis, mice at 8 weeks of age were intraperitoneally (i.p.) injected with vehicle (mineral oil, Sigma, M5310) or 10% CCl4 (Sigma, 319961) dissolved in mineral oil at 0.7 mL/kg twice a week for 4 weeks. For siRNA treatment studies, 8 wk-old mice were randomly divided into three groups: vehicle plus AAV-scr (group 1), CCl4 plus AAV-scr (group 2), and CCl4 plus AAV-siTET3 (group 3). AAV-siTET3 (Applied Biological Materials Inc., iAAV04929608) allows the expression of siRNAs specific for mouse TET3 from adeno-associated virus (serotype 8)-based vectors. AAV-scr (Applied Biological Materials Inc., iAAV01508) expresses non-targeting scrambled siRNAs as a negative control. Both viruses were resuspended in PBS/0.5% sorbital buffer. Vehicle or CCl4 was injected i.p. twice a week; AAV-scr or AAV-siTET3 at 2×10¹⁰ gc/mouse was injected via tail vein once a week. Five weeks following the initial injection, mice were sacrificed for blood and tissue collection.

STZ Model

The animal studies were approved by the Yale University Institutional Animal Care and Use Committee. All mice used in this study were male. The WT and H19 knockout (KO) mice on a background of C57BL/6J were gifts from Dr. Luisa Dandolo (Institut Cochin, Paris, France). Mice were housed at 22-24° C. with a 12 h light/12 h dark cycle with regular chow (Harlan Teklad no. 2018, 18% calories from fat) and water provided ad libitum. Type 1 diabetes was induced in 13-week old mice by intraperitoneal injection of 50 mg/kg body weight streptozotocin (STZ, Cat. 572201, EMD Millipore Corporation, USA) daily for 5 days. Blood glucose was monitored weekly. After two consecutive readings of >250 mg/dl (Breeze; Bayer Health Care LLC, Mishawaka, Ind.), mice were considered diabetic. Pulmonary endothelial cells were collected after 5 weeks of initial STZ injection.

Patient Liver Sample Collection

Fibrotic and non-fibrotic liver tissues were obtained from 12 patients who underwent liver cancer resection in Shengjing Hospital of China Medical University between May 2018 and September 2018. Ethics approval for this study was obtained from the Institutional Review Board at China Medical University, and written informed consent was obtained from each patient. All patients had a history of hepatitis B virus infection. Among the 12 patients, 7 were diagnosed with liver cancer with concurrent liver fibrosis (fibrosis group) and the other 5 were diagnosed with liver cancer without liver fibrosis (control group). Fibrotic liver tissues were collected from cancer-free background livers of the fibrosis group; non-fibrotic liver tissues were collected from cancer-free background livers of the control group. Tissues were snap frozen in liquid nitrogen and stored at −80° C. until use.

Histopathology Analysis

Mouse liver samples were collected and fixed at 4° C. with 4% of phosphate buffered paraformaldehyde (Baker, 2106-01) for 24 h. Fixed tissue specimens were embedded in paraffin and cut into 4 mm-thick tissue sections for histopathological analysis and immunohistochemistry (IHC). Archived deidentified paraffin embedded human liver tissue sections (fibrosis and normal control) were obtained from the Yale Pathology Tissue Services. Quantification of fibrosis was performed using ImageJ.

Cell Culture, Transfection, and Cytokine Treatment

LX-2 human hepatic stellate cells (Yale Liver Center) were maintained in DMEM (Gibco, 11965-092) supplemented with 10% heat-inactivated fetal bovine serum (Gibco, 26140-079) and 1% PS (penicillin and streptomycin) at 37° C. in 5% humidified CO2 tissue culture incubator. Generally, cells were plated and transfected in a 24-well plate scale. For transfection with siRNA, let-7, or iLet7, 10 pmol of siTET3 (or siCon), let-7 (or miCon), or iLet7 (iCon) were mixed with 25 μl of OPTI-MEM (Gibco, 31985-070) by gentle pipetting. In parallel, 1 μl of Lipofectamine 3000 (ThermoFisher Scientific, L3000015) was mixed with 25 μl of OPTI-MEM. Then, the contents from the two tubes were mixed by gentle pipetting. After incubation for 5 min at RT, the resulting 50 μl solution was used to resuspend cell pellet (5×10⁴ cells). After incubation at RT for 10 min, 450 μl of growth medium was added and the cell suspension was transferred into one well of a 24-well plate. For transfection with TET3-expression plasmid pTET3 (pcDNA-Flag-Tet3, Addgene plasmid #60940, (30)) or control vector, 5×10⁴ cells per well were seeded in a 24-well plate the night before transfection. The next day, 0.75 μg of pTET3 (or Vec) were mixed with 1 μl of P3000 in 25 μl of OPTI-MEM by gentle pipetting. In parallel, 0.5 μl of Lipofectamine 3000 were mixed with 25 μl of OPTI-MEM. The contents from the two tubes were combined by gentle pipetting and incubated for 5 min at RT. The resulting 50 μl of transfection solution was added to each well of cells seeded in a 24-well plate the night before and from which the growth medium had been removed. After 6 h of incubation in a tissue culture incubator, 500 μl of growth medium were added to each well of cells. For TGF-β1 (Sigma, USA) treatment experiments, transfected cells were incubated in 500 μl of serum-free DMEM containing 5 ng/mL TGF-β1 (diluted with HCl) or HCl (as control) for 24 h or 48 h as indicated.

HUVECs (Lonza, CC-2517) were maintained in a growth medium composed of endothelial basal medium (EBM-2, CC-3156) supplemented with EGM-2 MV BulletKit (CC-4147, Lonza). Cells were used between passages P2-P5. For cytokine treatment, cells were incubated overnight with indicated concentrations of TNF-α, TNF-α plus IL-1β, or Hcy in endothelial basal medium. Cells were transfected in a 24-well plate scale. To prepare siRNA transfection solution for each well, 10 pmol of siCon, siH19, or siTET1 were mixed with 25 μl of OPTI-MEM by gentle pipetting. In parallel, 1 μl of Lipofectamine 3000 was mixed with 25 μl of OPTI-MEM. Following 5 min of incubation at RT, the two were mixed by gentle pipetting and incubated for 10 min at RT to allow siRNA/lipid complexes to form. At the end of incubation, the 50 μl of transfection solution was used to resuspend cell pellet (3×10⁴ cells/well). After incubation at room temperature (RT) for 10 min, growth medium was added at a ratio of 1:9 (1 volume of transfection solution/9 volumes of growth medium) and the cell suspension was transferred to a culture plate. For cytokine treatment combined with H19 knockdown, cells were transfected with siCon or siH19 for 24 h, followed by addition of TNF-α at a final concentration of 10 ng/ml for an additional 24 h before RNA extraction. For iLet 7 rescue experiments, 10 pmol of siCon+iCon, iCon+siH19, or siH19+iLet7 were used for each well of 3×10⁴ cells. Forty-eight hours after the transfection, RNA and protein were extracted for analysis.

To prepare DNA/lipid complexes for pH19/pTET1 overexpression (FIG. 23) or pTET1 overexpression rescue (FIG. 22) experiments, empty vector (0.5 μg), pH19 (0.5 m), or pTET1 (0.75 μg) were mixed with 1 μl of P3000 in 25 μl of OPTI-MEM by gentle pipetting. In parallel, 0.75 μl of Lipofectamin 3000 was mixed with 25 μl of OPTI-MEM. The contents in the two tubes were mixed by gentle pipetting and incubated at RT for 5 min. The resulting DNA/lipid complexes (50 μl) were added to one well of cells (8×10⁴) which had been seeded in a 24-well plate in growth medium the night before. Following incubation at RT for 10 min, 300 μl of OPTI-MEM was added and cells were incubated in a tissue culture incubator for 4-6 h. Subsequently, 500 μl of growth medium was added and cells were incubated in a tissue culture incubator overnight. The next day, the medium was replaced with fresh growth medium. For pTET1 overexpression rescue experiments (FIG. 22), the indicated pTET1/lipid complexes and siH19/lipid complexes were prepared separately, and then pooled to one well of 8×10⁴ cells seeded the night before in a 24-well plate. Forty-eight hours after the transfection, RNA and protein were extracted for analysis.

PTT, GTT, and ITT

Pyruvate tolerance tests (PTT) and glucose tolerance tests (GTT) were performed following 12 h and 16 h overnight fasting, respectively. Each animal received an i.p. injection of 2 g/kg pyruvate (Sigma-Aldrich, cat #P5280) or 2 g/kg of glucose (Sigma-Aldrich, G5767) in sterile saline. Insulin tolerance tests (ITT) were performed following morning-fasting (3 h). Each animal received an i.p. injection of 1 U/kg insulin (Humulin R; Eli Lilly). Blood glucose concentrations were measured using Breeze2 glucometer (Bayer) via tail vein bleeding at the indicated time points after injection. Plasma insulin levels were measured using Mouse Insulin ELISA kit (EMD Millipore, Billerica, Mass., catalog #EZRMI-13K) according to the manufacturer's instructions. For all experiments, age-matched animals were used. For RNA and protein analyses, tissues were collected following euthanasia, snap frozen in liquid nitrogen, and stored at −80° C. until use. For information on animal numbers, refer to Figure legends.

Adenovirus (Ad) and Adeno-Associated Virus (AAV) Production and In Vivo Virus Administration

The Ad-GFP adenovirus was purchased (CV10001, Vigene Biosciences Inc.). The Ad-TET3 virus was custom made by cloning a mouse full-length TET3 ORF PCR amplified from pcDNA-Flag-Tet3 (Wang Y et al., 2014, Cell Rep 6:278-284) (60940, Addgene) into an Ad-TBG vector that drives TET3 expression from a liver-specific thyroxine binding globulin (TBG) promoter (Vigene Biosciences, Rockville, Md. 20850 USA). The AAV-H19 virus was previously described (Zhang N et al., 2018, JCI Insight 3:e120304). The negative control siRNA virus AAV-scr was purchased (iAAV01500, Applied Biological Materials Inc.). The AAV-siTET3, AAV-siP2, AAV-sihTET3, and AAV-sihP2 viruses were custom made by Applied Biological Materials Inc. All AAV viruses were serotype 8. The targeted sequence for AAV-siTET3 is 5′-CGGTACCATCTCCTATTTCTCAGAGGGAG, for AAV-siP2 is 5′-CCTTTGCTGCTGTGTGTGGGCCCCTGCTC, for AAV-sihTET3 is 5′-GCCCACAAGGACCAGCATAACCTCTACAA, and for AAV-sihP2 is 5′-ATGGTCAGCGTGAACGCGCCCCTCGGGGC.

Mice were tail vein injected with indicated viruses at 1×10¹⁰ gc/mouse (adeno) or 2×10′ gc/mouse (AAV), in 150 μl of PBS/0.5% sorbitol. Before injection, mice were exposed to heat lamp to dilate the tail vein and then placed in a restrainer permitting access to the tail vein. The tail was cleansed with 70% ethanol and the injection was made in the lateral vein, using 30-gauge needles.

Primary Hepatocytes and Virus Infection

Mouse primary hepatocytes were prepared as previously described (Zhang N et al., 2018, JCI Insight 3:e120304). Cells were maintained in Williams' Medium (GIBCO, 12551) with 5% FBS, 1% 1 M HEPES buffer (GIBCO, 15630-080), 1% L-Glutamine (GIBCO, 25030-081), 1% SPA (GIBCO, 15240-062), 4 mg/L insulin (GIBCO, 12585-014) and 1 μM dexamethasone (Sigma, D4902). For TET3 overexpression experiments, freshly isolated mouse primary hepatocytes were seeded in 12-well collagen 1-coated plates (BD Biosciences Discovery Labware) at 4×10⁵ cells/well and infected with Ad-GFP or Ad-TET3 at 4,000 gc/cell at 3 h after seeding. For TET3 or HNF4α P2 isoform knockdown experiments, cells seeded in 12-well plates were infected with AAV-scr, AAV-siTET3, or AAV-siP2 at 6,000 gc/cell at 3 h after seeding. Medium was changed the next day and glucagon was added at a final concentration of 20 nM. RNA and protein were isolated at the time points indicated in the Figure legends. Glucose production assays were carried out at 60 h after infection. Human primary hepatocytes (M00995, Millipore, Sigma) were purchased, thawed, and maintained in InVitroGRO™ CP Medium (Sigma-Aldrich, Z990003) containing 2% Torpedo™ Antibiotic Mix. The cells were seeded in 12-well plates coated with collagen 1 (Millipore, 08-115). Infection of AAV-scr, AAV-sihTET3, and AAV-sihP2 were performed the next day after cell thawing and followed the same dosing protocol for the mouse primary hepatocytes.

Luciferase Reporter Assays

Human U-2 OS cell line (92022711) was purchased as authenticated from Sigma Aldrich and maintained in McCoy's 5a medium supplemented with 1.5 mM glutamine and 10% FBS. The assays were carried out in a 48-well plate scale as previously described (Qiu C et al., 2010, Nucleic Acids Res 38:1240-1248) with minor modifications. Transfections were performed with lipofectamine 3000 (Thermo Fisher Scientific) with a fixed total quantity of DNA (300 ng per well). 100 ng of gAF1 (Pck1 firefly luciferase reporter) and 0.2 ng of Renilla luciferase construct (for transfection efficiency normalization) (Sharabi K et al., 2017, Cell 169:148-160), together with 0, 10, 20, 40, or 80 ng of pAd-Track Flag HA PGC-1 alpha (expresses PGC-1α, #14426, Addgene) (Lerin C et al., 2006, Cell Metab 3:429-438) and 25 ng of FR HNF4A8 (expresses HNF4α8, #31114, Addgene) (Erdmann S et al., 2007, Biol Chem 388:91-106) or FR HNF4A2 (expresses HNF4α2, #31100, Addgene) (Thomas H et al., 2004, Nucleic Acids Res 32:e150) were transfected into U-2 OS cells. To prepare transfection solution for each well, plasmids were mixed with 25 μl OPTI-MEM and 1 μl P3000 by gentle pipetting. In parallel, 0.75 μl Lipofectamine 3000 was mixed with 25 μl OPTI-MEM. Following 5 min of incubation at room temperature (RT), the two were mixed by gentle pipetting and incubated for 10 min at RT to allow plasmids/lipid complexes to form. At the end of incubation, the 50 μl transfection solution was used to re-suspend cell pellet (4×10⁴ cells). After incubation at RT for 10 min, regular growth medium was added at a ratio of 1:5 (1 volume of transfection solution/5 volumes of growth medium) and the cell suspension was transferred to one well of a 48-well plates. Experiments were run in triplicate. Luciferase activities were measured 24 h post-transfection using Promega Dual-Luciferase Reporter Assay System (E1960) according to the manufacturer's protocol. Data are presented as firefly luciferase reporter values normalized to Renilla values.

Tissue Sample Collection for Example 3

Paired fibroid and myometrial tissues were collected from 30 women (age=/<50, premenopausal, n=20; age >50, postmenopausal, n=10) who had a hysterectomy or myomectomy for uterine fibroids in Shengjing Hospital of China Medical University between December 2017 and January 2018. Ethics approval for this study was obtained from the Institutional Review Board at China Medical University, and all patients signed informed consent. No women received hormones within 3 months prior to surgery. For premenopausal women, fibroid and myometrial tissues were collected at the proliferative stage of the menstrual cycle. Normal myometrium tissues were taken at a distance of 2-cm from adjacent fibroids. When multiple fibroids were present, a sample was taken from the body of the largest fibroid. Fibroids were not selected according to their locations (intramural, subserosal, or submucosal); however, degenerative fibroids were excluded. Tissues were snap frozen in liquid nitrogen and stored at 80° C. until use.

Tissue Real-Time PCR Array for Example 3

Frozen tissue samples were homogenized in liquid nitrogen, followed by RNA extraction using TRIzol Reagent (15596026, ThermoFisher Scientific) according to the manufacturer's protocol. DNA contamination was removed by DNase I digestion (AM2222, ThermoFisher Scientific). Total RNA was reverse-transcribed using the PrimeScript™ RT Reagent Kit with gDNA Eraser (TaKaRa, Dalian, China) and amplified by GoTaq® qPCR Master Mix (Promega, Madison, Wis., USA) in the ABI ViiA 7 Real-time PCR system (Applied Biosystems, USA). The threshold cycle (Ct) values of each sample were used in the post-PCR data analysis. Gene expression levels were normalized against GAPDH.

Primary Cell Isolation and Culture

Fibroid tissues were minced and digested in Hanks' balanced salt solution containing 1% penicillin, 1% streptomycin, 1% collagenase (Sigma-Aldrich: COLLD-RO ROCHE 11088866001), and 0.01% deoxyribonuclease I (Sigma-Aldrich 10104159001) at 37° C. for 40 min, with gentle vortexing every 10 min. Dispersed cells were filtered through Fisherbrand Sterile 70 um Nylon Mesh Cell Strainers (Fisher scientific, 22-363-548). The resulting cells in single cell suspension were seeded onto 10-cm tissue culture dishes in DMEM medium (10567014, Gibco) containing 20% fetal bovine serum (Gibco, 26140-079), maintained at 37° C. in a humidified atmosphere (5% CO2 in air), and grown to confluence. Cells were used between passages P1-P5.

Isolation of Pulmonary Endothelial Cells

Primary mouse pulmonary endothelial cells were isolated with rat anti-mouse CD31 antibody (clone MEC 13.3, Pharmingen, #553370) conjugated to Dynabeads (catalog number 110.35, Invitrogen) using a protocol similar to that previously described (Lanahan et al, 2010, Dev Cell 18:713-724).

Leiomyoma Cell Culture and Transfection

ht-UtLM cells tested negative for mycoplasma contamination were maintained in MEM (Sigma, #M3024) supplemented with 20% fetal bovine serum, 1×MEM Vitamin Solution (Invitrogen #11120-052), 1×MEM Amino Acids Solution (Invitrogen, #11130-051), and 1×MEM Non-Essential Amino Acids (Invitrogen #11140-050). Primary UtLM-1 and UtLM-2 cells were maintained in DMEM (Gibco, #10567014) supplemented with 20% fetal bovine serum, and 1% penicillin/streptomycin, 1% amphotericin B. All cells were transfected in a 24-well plate scale. To prepare siRNA transfection solution for each well, 10 pmol (5 pmol for primary cells) of siCon, siH19, or siTET3 were mixed with 25 μl of OPTI-MEM by gentle pipetting. In parallel, 1 μl of Lipofectamine 3000 was mixed with 25 μl of OPTI-MEM. Following 5 min of incubation at room temperature, the two were mixed by gentle inverting and incubated for 10 min at room temperature to allow siRNA/lipid complexes to form. At the end of incubation, the 50 μl of transfection solution was used to re-suspend the cell pellet (3×10⁴ cells). After incubation at room temperature for 10 min, growth medium was added at a ratio of 1:9 (1 volume of transfection solution/9 volumes of growth medium), and the cell suspension was transferred to a new culture dish. After overnight incubation in a tissue culture incubator, the medium was replaced with fresh growth medium. For iLet 7 rescue experiments, 10 pmol of siCon/siH19 and 10 pmol of iCon/iLet7 were used for each well of 3×10⁴ cells. RNA, genomic DNA, and protein were extracted and analyzed at the indicated time points following transfection.

Cell Viability Analysis for Example 3

These were performed as previously described (Yan L et al., 2015, Oncogene 34:3076-3084). Briefly, cells were transfected and seeded in 96-well plates at a density of 4×10³/well. Cell viability and caspase 3/7 activity were measured 48 h post-transfection using the CellTiter-Blue Cell Viability kit (Promega) and the Apo-ONE Homogeneous Caspase-3/7 Assay kit (Promega), respectively, according to the manufacturer's protocols.

Quantitative Methylation Specific PCR (QMSP) for Example 1

Genomic DNA was extracted from mouse primary hepatocytes grown in 12-well plates using Quick-gDNA MicroPrep (Zymo Research Corporation, D3021) according to the manufacturer's instructions. For bisulfite treatment, 480 ng of DNA was used for each column using EZ DNA Methylation-Gold Kit (Zymo, D5006). 100 μl of elution buffer was used to elute DNA from each column. Real-time quantitative PCR was performed in a 15 μl reaction containing 5 μl of the eluted DNA using iQSYBRGreen in a Bio-Rad iCycler. Two sets of PCR primers were designed: one for unmethylated and one for methylated DNA sequences. The PCR primers for methylated DNA were used at a final concentration of 0.6 μM in each PCR reaction. PCR was performed by initial denaturation at 95° C. for 5 min, followed by 40 cycles of 30 sec at 95° C., 30 sec at 60° C., and 30 sec at 72° C. Specificity was verified by melting curve analysis. The threshold cycle (Ct) values of each sample were used in the post-PCR data analysis. The ratio of methylated versus unmethylated DNA sequences are presented. The primers used for QMSP are listed in Table 1.

TABLE 1
List of Primers used for QMSP.
Real-time PCR primer sequences (mouse)

| Gene | Forward Primer | Reverse Primer |
|---|---|---|
| Rplp0 | 5′-GATGGGCAAC | 5′-CTGGGCTCCT |
| | TGTACCTGAC | CTTGGAATG-3′ |
| | TG-3′ | |
| Hprt1 | 5′-CAGTCCCAGC | 5′-GGCCTCCCAT |
| | GTCGTGATTA-3′ | CTCCTTCATG- |
| | | 3′ |
| Tubulin(H/m) | 5′-CGTGTTCGGC | 5′- |
| | CAGAGTGGTG | GGGTGAGGGC |
| | C-3′ | ATGACGCTGA |
| | | A-3′ |
| Gapdh | 5′-CCTTCATTGA | 5′-CAAAGTTGTC |
| | CCTCAACTAC | ATGGATGACC |
| | AT-3′ | -3′ |
| ACTB | 5′-GTGGGCCGCT | 5′- |
| | CTAGGCACCA | CTCTTTGATG |
| | A-3′ | TCACGCACGA |
| | | TTT-3′ |
| Tet1 | 5′-AAGAAGAGGA | 5′-GGCCATTTAC |
| | AATGCGAGGT-3′ | TGGTTTGTTG- |
| | | 3′ |
| Tet2 | 5′-AGCAAGAGAT | 5′-AGTGGAGGAC |
| | TCCGAAGGAT-3′ | TGAGTGCAAG |
| | | -3′ |
| Tet3 | 5′-TGCGATTGTG | 5′-TCCATACCGA |
| | TCGAACAAAT | TCCTCCATGA |
| | AGT-3′ | G-3′ |
| Tgfb1 | 5′-GGAATACAGG | 5′-CTCTGTGGAG |
| | GCTTTCGATT-3′ | CTGAAGCAAT-3′ |
| TgfβR1 | 5′-GGAATACAGG | 5′-CTCTGTGGAG |
| | GCTTTCGATT-3′ | CTGAAGCAAT-3′ |
| TgfβR2 | 5′-AGCGGGGAAT | 5′-GAGGAATGAC |
| | TTACAGAATG-3′ | AGCGATGCTA-3′ |
| Tsp1 | 5′-AAAGCCAAAG | 5′-TGGCGGTGAG |
| | CGCCTATTTA-3′ | TTCTAGTGAG-3′ |
| Acta2 | 5′-CTGGCCTAGC | 5′-GTGGACCTTC |
| | AACACTGATT-3′ | CTCTGTTGAA-3′ |
| Col1a1 | 5′-ATGATGCTAA | 5′-TGGTTAGGGT |
| | CGTGGTTCGT-3′ | CGATCCAGTA-3′ |
| Fn1 | 5′-GACAGTTGGT | 5′-TGACTTTCCT |
| | CACCCTGTTC-3′ | GCTCAAGGTC-3′ |
| Timp1 | 5′-CCGCAGTGAA | 5′-TCACTCTCCA |
| | GAGTTTCTCA-3′ | GTTTGCAAGG-3′ |
| | 5′--3′ | 5′--3′ |
| | 5′--3′ | 5′--3′ |

Real-time PCR Primer sequences (human)

| Gene | Forward Primer | Reverse Primer |
|---|---|---|
| HPRT1 | 5′-GACCAGTCAA | 5′-CCTGACCAAG |
| | CAGGGGACAT-3′ | GAAAGCAAAG-3′ |
| RPLP0 | 5′-GGCGACCTGG | 5′-CCATCAGCAC |
| | AAGTCCAACT-3′ | CACAGCCTTC-3′ |
| TUBB | 5′-CGTGTTCGGC | 5′-GGGTGAGGGC |
| | CAGAGTGGTG | ATGACGCTGA |
| | C-3′ | A-3′ |
| β-actin | 5′-ATCAAGATCA | 5′-CTGCTTGCTG |
| | TTGCTCCTCC | ATCCACATCT |
| | TGAG-3′ | G-3′ |
| GAPDH | 5′-CTTTGTCAAG | 5′-TCTTCCTCTT |
| | CTCATTTCCT | GTGCTCTTGC-3′ |
| | GG-3′ | |
| U6 | 5′-GCTCGCTTCG | 5′-AACGCTTCAC |
| | GCAGCACA-3′ | GAATTTGCGT |
| | | G-3′ |
| Lin28B | 5′-AAAACAGAAC | 5′-GTAATGGCAT |
| | ACGGAAGCAG-3′ | TGCATGTTCA-3′ |
| TET1 | 5′-GCAGCGTACA | 5′-AGCCGGTCGG |
| | GGCCACCACT-3′ | CCATTGGAAG-3′ |
| TET2 | 5′-TTCGCAGAAG | 5′-AGCCAGAGAC |
| | CAGCAGTGAA | AGCGGGATTC |
| | GAG-3′ | CTT-3′ |
| TET3 | 5′-GACGAGAACA | 5′-GTGGCAGCGG |
| | TCGGCGGCGT-3′ | TTGGGCTTCT-3′ |
| TGFB1 | 5′-CAAGCAGAGT | 5′-TGCTCCACTT |
| | ACACACAGCA | TTAACTTGAG |
| | T-3′ | CC-3′ |
| TGFBR2 | 5′-GGTTCCTGTG | 5′-TGCAACCCAT |
| | TGCCCTTATT-3′ | GAAGGTAAAA-3′ |
| TSP1 | 5′-AGCGTCTTCA | 5′-CATTCACCAC |
| | CCAGAGACCT-3′ | GTTGTTGTCA-3′ |
| ACTA2 | 5′-AGTTACGAGT | 5′-GAGGTCCTTC |
| | TGCCTGATGG-3′ | CTGATGTCAA-3′ |
| COL1A1 | 5′-GGACACAGAG | 5′-CCAGTAGCAC |
| | GTTTCAGTGG-3′ | CATCATTTCC-3′ |
| FN1 | 5′-AGTGGGAGAC | 5′-ACTGTGACAG |
| | CTCGAGAAGA-3′ | CAGGAGCA-3′ |
| TIMP1 | 5′-TACTTCCACA | 5′-ATTCCTCACA |
| | GGTCCCACAA-3′ | GCCAACAGTG-3′ |

ChIP primer sequences (human)

| Gene | Forward Primer | Reverse Primer |
|---|---|---|
| TGFB1 | 5′-CCTGCCGACC | 5′-CTCGCTGTCT |
| | CAGCC-3′ | GGCTGCT-3′ |
| TGFBR2 | 5′-GGGCTGGTCT | 5′-GAAACAGGAA |
| | AGGAAACATG | ACTCCTCGCC |
| | ATTGG-3′ | AACA-3′ |
| TSP1 | 5′-GCCCATTGGC | 5′-CTGAACTCGC |
| | CGGAGGAATC-3′ | AGGCCAGCTC |
| | | G-3′ |

QMSP primer sequences (human)

| Gene | Forward Primer | Reverse Primer |
|---|---|---|
| TGFBR2 | 5′-GGAGAAGGGA | 5′-AATAACTCAC |
| (methylated) | GAAGGTTTTC | TCAACTTCAA |
| | G-3′ | CTCAAC-3′ |
| TGFBR2 | 5′-AGGAGAGGGA | 5′-AATAACTCAC |
| (unmethylated) | GAAGGTTTTT | TCAACTTCAA |
| | G-3′ | CTCAAC-3′ |
| TSP1 | 5′-CGAGTTTAGG | 5′-AAAACGACTT |
| (methylated) | GTTTTTG-3′ | ACCTATATAT |
| | | ACCGAAA-3′ |
| TSP1 | 5′-TGTGAGTTTA | 5′-AAAACGACTT |
| (unmethylated) | GGGTTTTTGT | ACCTATATAT |
| | TG-3′ | ACCGAAA-3′ |
| TGFB1 | 5′-GGAGAAGGGA | 5′-AATAACTCAC |
| (methylated) | GAAGGTITTC | TCAACTTCAA |
| | G-3′ | CTCAAC-3′ |
| TGFB1 | 5′-TTTGGGTTAT | 5′-ACTCTAAAAC |
| (unmethylated) | TTTTTTTTTA | CTCAAACTAC |
| | TTTTTTTTCG | TCCT-3′ |
| | C-3′ | |

TET3 siRNA sequence (human)
sense 5′-CAGCAACUCCUAGAACUGAtt-3′,
antisense 5′-UCAGUUCUAGGAGUUGCUGga-3′

Quantitative Methylation-Specific PCR (QMSP) for Example 2

The assays were carried out as previously described in (Zhou J et al., 2015, Nat Commun 6:10221; Zhong T et al., 2016, Oncogene 36:2345-2354) with minor modifications. Briefly, gDNA was extracted from LX-2 cells in 24-well plates using Quick-gDNA MicroPrep (Zymo Research Corporation, Irvine, Calif.; D3021). For bisulfite treatment using EZ DNA Methylation-Gold Kit (Zymo, D5006), 90-500 ng of gDNA was used per column and 20-40 μl of elution buffer was used to elute DNA from each column. qPCR was performed in a 15 μl reaction containing 5 μl of eluted DNA using iQSYBRGreen (Bio-Rad, Hercules, Calif.; 1708880) in a Bio-Rad iCycler. PCR was conducted by initial denaturation at 95° C. for 5 min, followed by 40 cycles of 30 sec at 95° C., 30 sec at 60° C., and 30 sec at 72° C. Two sets of PCR primers were designed: one for unmethylated and one for methylated DNA sequences. The PCR primers for methylated DNA were used at a final concentration of 0.6 μM in each PCR reaction. The relative levels of methylated versus unmethylated DNA sequences are presented. The primers used for QMSP are summarized in Table 1.

Quantitative Methylation-Specific PCR (QMSP) for Example 3

gDNA was extracted from primary leiomyoma cells from one well of 24-well plates using Quick-gDNA MicroPrep (Zymo Research Corporation, Irvine, Calif.; D3021) according to the manufacturer's instructions. For bisulfite treatment, 200 ng of gDNA was used for each column using EZ DNA Methylation-Gold Kit (Zymo, D5006). 100 μl of elution buffer was used to elute gDNA from each column. Real-time quantitative PCR was performed in a 15 μl reaction containing 5 μl of the eluted gDNA using iQSYBRGreen (Bio-Rad, Hercules, Calif.; 1708880) in a Bio-Rad iCycler. Two sets of PCR primers were designed: one for unmethylated and one for methylated DNA sequences. The PCR primers for methylated DNA were used at a final concentration of 0.6 μM in each PCR reaction. PCR was performed by initial denaturation at 95° C. for 5 min, followed by 40 cycles of 30 sec at 95° C., 30 sec at 60° C., and 30 sec at 72° C. Specificity was verified by melting curve analysis and agarose gel electrophoresis. The threshold cycle (Ct) values of each sample were used in the post-PCR data analysis. The relative levels of methylated versus unmethylated DNA sequences are presented.

Quantitative Methylation-Specific PCR (QMSP) for Example 4

Genomic DNA was extracted from HUVECs in one well of 24-well plates using Quick-gDNA MicroPrep (Zymo Research Corporation, Irvine, Calif.; D3021) according to the manufacturer's instructions. For bisulfite treatment, 200 ng of DNA was used for each column using EZ DNA Methylation-Gold Kit (Zymo, D5006). 100 μl of elution buffer was used to elute DNA from each column. Real-time quantitative PCR was performed in a 15 μl reaction containing 5 μl of the eluted DNA using iQSYBRGreen (Bio-Rad, Hercules, Calif.; 1708880) in a Bio-Rad iCycler. Two sets of PCR primers were designed: one for unmethylated and one for methylated DNA sequences. The PCR primers for methylated DNA were used at a final concentration of 0.6 μM in each PCR reaction. PCR was performed by initial denaturation at 95° C. for 5 min, followed by 40 cycles of 30 sec at 95° C., 30 sec at 60° C., and 30 sec at 72° C. Specificity was verified by melting curve analysis and agarose gel electrophoresis. The threshold cycle (Ct) values of each sample were used in the post-PCR data analysis. The ratio of methylated versus unmethylated DNA sequences are presented.

Chromatin Immunoprecipitation-Quantitative PCR (ChIP-qPCR) for Example 1

Experiments were performed in a 12-well plate scale using the Pierce Agarose ChIP Kit (Thermo Scientific, 26156) according to the manufacturer's instructions with minor modifications. Briefly, agarose beads were used to pre-bind with antibodies (anti-TET3, anti-Ser-5(P)-RNAP, or pre-immune IgGs as negative controls) overnight at 4° C. The next day, hepatocytes were crosslinked with 1% formaldehyde at room temperature for 10 min, and the reaction was stopped by 1×glycine. ChIPs were carried out overnight at 4° C. Levels of ChIP-purified DNA were determined with qPCR (see Table 1 for primer sequences). The relative enrichments of the indicated DNA regions were calculated using the Percent Input Method according to the manufacturer's instructions and are presented as % input.

Chromatin Immunoprecipitation-Quantitative PCR (ChIP-qPCR) for Example 2

These experiments were performed as previously described (Zhou J et al., 2015, Nat Commun 6:10221) with minor modifications. Briefly, experiments were performed in a 10-cm plate scale using Chromatin Prep Module Kit (Thermo, catalog number 26158) according to the manufacturer's instructions. Agarose beads were used to pre-bind overnight with antibodies against TET3 (Millipore Sigma, ABE290), H3K4me3 (Cell signaling, C42D8) and H3K27me3 (Cell signaling, C36B11). Preimmune IgG was used as a negative control. LX-2 cells were cross-linked with 1% formaldehyde at RT for 10 min and the reaction was stopped by 1× glycine. ChIPs were carried out overnight at 4° C. Primers (Table 1) for the specific promoter regions of TGFB1, TSP1, and TGFBR2 were used to amplify input and ChIP-purified DNA. The relative enrichments of the indicated DNA regions were calculated using the Percent Input Method according to the manufacturer's instructions and were normalized to % input. Data are presented after normalization against background IgG signals.

Chromatin Immunoprecipitation-Quantitative PCR (ChIP-qPCR) for Example 3

These experiments were performed as previously described (Zhou J et al., 2015, Nat Commun 6:10221) with minor modifications. Briefly, experiments were performed in a 10-cm plate scale using the Pierce Agarose ChIP Kit (Thermo Scientific, 26156) according to the manufacturer's instructions with minor modifications. Agarose beads were used to pre-bind overnight with antibodies against TET3 (Millipore Sigma, ABE290), H3K4me3 (Cell signaling, C42D8) and H3K27me3 (Cell signaling, C36B11). Preimmune IgG was used as a negative control. Cells were cross-linked with 1% formaldehyde at room temperature for 10 min, and the reaction was stopped by 1× glycine. ChIPs were carried out overnight at 4° C. Primers for the specific promoter regions of MED12, TGFBR2, and TSP1 (FIG. 16A) were used to amplify input and ChIP-purified DNA. The relative enrichments of the indicated DNA regions were calculated using the Percent Input Method according to the manufacturer's instructions and were normalized to % input. Data are presented after normalization against background IgG signals.

Chromatin Immunoprecipitation-Quantitative PCR (ChIP-qPCR) for Example 4

Experiments were performed in a 10-cm dish scale using the Pierce Agarose ChIP Kit (Thermo Scientific, 26156) according to the manufacturer's instructions with minor modifications. Briefly, agarose beads were used to pre-binding overnight with antibodies against TET1 (Millipore Sigma, ABE1034), H3K4me3 (Cell signaling, C42D8) and H3K27me3 (Cell signaling, C36B11). IgG was included as a negative control. Cells were cross-linked with 1% formaldehyde at room temperature for 10 min, and the reaction was stopped by 1× glycine. ChIPs were carried out overnight at 4° C. Primers for the promoter regions of TGFBR2 and TSP1 were used to amplify input and ChIP-purified DNA. The relative enrichments of the indicated DNA regions were calculated using the Percent Input Method according to the manufacturer's instructions and were normalized to % input.

Glucose Production Assay

Glucose production assays were performed as previously described (Zhang N et al., 2018, JCI Insight 3:e120304) using Amplex Red Glucose/Glucose Oxidase Assay Kit (Molecular Probes, Invitrogen, A22189), according to the manufacturer's instructions. Briefly, primary hepatocytes grown in 12-well plates were used. On the day of the assay, culture medium was replaced with glucose-free and phenol red-free DMEM (Gibco, A14430-01) supplemented with 2 mM L-glutamine and 15 mM HEPES for 2 h. Then, cells were incubated in 120 μl of glucose production medium (glucose-free and phenol red-free DMEM, 20 mM sodium lactate, 2 mM sodium pyruvate, and 0.5% BSA, 2 mM L-glutamine and 15 mM HEPES) for 4 h. Supernatants (50 μl) were used for measurements of glucose concentration, which was normalized to total protein content of cells.

RNA Extraction and RT-qPCR

Total RNAs were extracted from liver or pancreas tissues or from primary hepatocytes using PureLink RNA Mini Kit. cDNA was synthesized using PrimeScript RT Reagent Kit in a 20 μl reaction containing 0.5-1 μg of total RNA. Real-time quantitative PCR was performed in a 15 μl reaction containing 0.5-1 μl of cDNA using iQSYBRGreen in a Bio-Rad iCycler. PCR was performed by initial denaturation at 95° C. for 5 min, followed by 40 cycles of 30 sec at 95° C., 30 sec at 60° C., and 30 sec at 72° C. Specificity was verified by melting curve analysis. The Ct values of each sample were used in the post-PCR data analysis. Gene expression levels were normalized against house-keeping genes GAPDH and RPLP0. Real-time PCR primers are listed in Table 1.

Western Blot Analysis

Primary hepatocytes in 12-well plates were dissociated with 0.25% trypsin. After 405 centrifugation at 1000 rpm for 5 min, supernatants were discarded and cell pallets were homogenized in 2×SDS-sample buffer (100 ul/well), followed by heating at 100° C. for 5 min, with occasional vortexing. For liver tissue samples, 5 mg of tissues were minced and 200 μl of 2× SDSsample buffer was added, followed by homogenization on ice using a sonicator (Qsonica, Q125-110). Homogenized samples were heated at 100° C. for 5 min and then centrifuged at 12,000 g for 5 min to remove insoluble materials before loading onto 12% SDS gels (5 μl/well), followed by Western blot analysis. Bands on Western blot gels were quantified using ImageJ. GAPDH was used as a loading control.

Western Blot Analysis for Example 4

HUVECs in 24-well plates were dissociated with 0.25% trypsin. Cell pellets were collected by centrifugation at 1000 rpm for 5 min. Cell pellets were homogenized in 2×SDS-sample buffer (100 μl/well), followed by heating at 100° C. for 5 min, with occasional vortexing. Homogenized samples were loaded onto 12% SDS gel (5 μl/well), followed by Western blot analysis. Bands on Western blot gels were quantified using ImageJ. GAPDH was used as a loading control. For pulmonary endothelial cells, 2×SDS-sample buffer was used to homogenize the cells (50 μl/mouse).

RNA in situ Hybridization (RNA ISH)

H19 and CD31 RNA colocolization ISH experiments were performed on 5-μm frozen sections using the RNA scope 2.5 HD Duplex Reagent Kit (Advanced Cell Diagnostic Inc, 322500) according to the manufacturer's instructions. The system included the following components: Hs-H19 probe (P/N: 400771), Hs-PECAM-1 probe (P/N: 487381), Hs-Positive Control probe (P/N: 321641), 2-Plex Negative Control probe (P/N: 320751), Pretreatment Kit with protease IV (P/N: 322336), and Detection Kit (P/N: 322500). Briefly, samples were fixed with 4% formaldehyde for 2 h at RT, followed by protease IV digestion for 30 min to remove proteins including nucleases. The previously characterized H19 probe (Jiang et al, 2018, Hepatology communications 0:1-13; Zhang Z et al, 2016, J Clin Pathol 69:76-81) and CD31 probe were mixed and added to the slides to hybridize for 2 h at 40° C. in a HybEz oven (Advanced Cell Diagnostic Inc), followed by signal amplification and washing steps, and mounted with VECTASHIELD Mounting medium (H-5000). Hybridization signals were detected by red and green chromogens for CD31 and H19, respectively. Images were acquired using the microscopy system (KEYENCE BZ-X710) in bright field mode.

RNA Extraction and RT-qPCR Assays

Total RNA was extracted from cultured cells or liver tissue samples using PureLink RNA Mini Kit (Ambion, 12183018A). 0.8 μg of total RNA was reverse transcribed to cDNA in a reaction volume of 10 μl using PrimeScript RT Reagent Kit (TAKARA, RR037A). Quantitative real-time PCR reactions were carried out using iQSYBRGreen (Bio-Rad) in a Bio-Rad iCycler. Gene expression levels were normalized against housekeeping genes HPRT1 and RPLP0. The specific PCR primers for human and mouse were summarized in Table 1.

RNA Extraction and RT-qPCR for Example 4

Total RNAs were extracted using PureLink RNA Mini Kit (Ambion, catalog number 12183018A). cDNA was synthesized using PrimeScript RT Reagent Kit (TAKARA, RR037A) in a 20 μl reaction containing 0.5-1 μg of total RNA. Real-time quantitative PCR was performed in a 15 μl reaction containing 0.5-1 μl of cDNA using iQSYBRGreen (Bio-Rad) in a Bio-Rad iCycler. PCR was performed by initial denaturation at 95° C. for 5 min, followed by 40 cycles of 30 sec at 95° C., 30 sec at 60° C., and 30 sec at 72° C. Specificity was verified by melting curve analysis and agarose gel electrophoresis. The threshold cycle (Ct) values of each sample were used in the post-PCR data analysis. Gene expression levels were normalized against Gapdh.

Serum TNF-α Analysis

Blood samples were collected by retro-orbital sinus puncture after 2 weeks of initial STZ injection via the medial cants of the eye using clean 100 μl of VWR Disposable Micropipets (Cat. 53432-921, VWR). After incubation on ice for 30 min, serum was obtained through centrifugation of the blood for 20 min at 3000 g at 4° C. and stored at −80° C. until use. Serum TNF-α measurements were performed using Luminex assays according to the manufacturers' protocols. Briefly, wells of a 96-well filter plate were loaded with either 50 μl of standard solution or 50 μl of serum and incubated with a mouse 23plex ProBead mix from BioRad (#m60009rdpd Bio-Rad Laboratories) at ±800 rpm for 30 min in the dark RT. Wells were then vacuum-washed three times with 100 μl of wash buffer. Samples were then incubated with 25 μl of biotinylated detection antibody at ±800 rpm for 30 min at RT in the dark. After three washes, 50 μl of streptavidin-phycoerythrin was added to each well and incubated for 10 min at ±800 rpm at RT in the dark. After a final wash, the beads were resuspended in 125 μl of sheath buffer for measurement using the LUMINEX 200 (LUMINEX, Austin, Tex., USA).

Immunofluorescence (IF)

Acquisition and processing of patient coronary artery specimens were previously described (Chen et al, 2015, J Clin Invest 125:4514-4528). All experiments were performed on frozen specimens (n=15) in Optimal Cutting Temperature Medium (Sakura Finetek USA Inc). Briefly, frozen tissue sections cut at 5-μm were washed 5 min x 3 and incubated with primary antibodies diluted in blocking solution (10% BSA and 5% horse serum in 1×PBS) overnight at 4° C. in a humidified chamber. Sections were washed for 10 min x 3 with 1×PBS, followed by incubation with second antibodies conjugated with Alexa Fluor 488 or Alexa Fluor 647 (diluted at 1:500 in blocking solution) for 1 h at RT. Sections were washed again for 10 min x 3 with 1×PBS, stained with DAPI (Vector Lab, Burlingame, Calif., diluted at 1:1000) for 5 min, and mounted with VECTASHIELD Mounting medium (H-1000). Images were obtained using the fluorescence microscopy system (KEYENCE BZ-X710) at a magnification of 40×. Image J was used for quantification. Measurements were made of the intima (I) and media (M) thickness. The I/M ratio was used to grade the atherosclerosis severity. Coronary arteries with an I/M ratio of less than 0.2 were considered as no disease or mild disease; those with an I/M ratio of 0.2 to 1 were considered as moderate disease; those with an I/M ratio of greater than 1 or with calcification were considered as having severe disease.

Immunoblotting Analysis

For liver tissue samples, 5-10 mg of tissues were homogenized by sonication (Qsonica, Model Q125) in 150 μl of 2×SDS-sample buffer. Samples were immediately heated at 100° C. for 3 min with occasional vertexing. Then, 100 μl of 2×SDS-sample buffer was added to each sample and the 250 μl of tissue lysate were further homogenized by sonication and heated again at 100° C. for 3-5 min with vertexing once every 15 sec. Samples (further diluted at 1:1 with 2×SDS-sample buffer before loading) were loaded 5-10 μl per well onto a 4-15% gradient SDS gel, followed by Western blot analysis. Image J was used to quantify the protein bands. The antibodies and Western blot conditions are listed in Table 2.

TABLE 2
List of Antibodies and Western Blot Conditions.

| LX-2 | Blocking | 1st Ab | 1st Ab | 1st Ab | 2nd Ab | 2nd Ab | 2nd Ab | Transfer | |
|---|---|---|---|---|---|---|---|---|---|
| (human) | 1 h | 4° C. O/N | dilution | Vendor | RT 2 h | dilution | Vendor | on ice | Wash |
| TET3 | 5% | 5% | 1:500 | GENE | 5% milk | Anti- | | 0.45 um | Wash 3 times after 1st |
| | nonfat | nonfat | | Tex | | Rabbit | | 100 V, | and 2nd Abs, 5 min each |
| | milk | milk | | 121453 | | IgG, | | 120 min | time |
| | | | | | | 1:4000 | | | |
| Col1A1 | 5% | 5% | 1:500 | Santa | 5% milk | Anti- | | 0.45 um | Wash 3 times after 1st |
| | nonfat | nonfat | | cruz sc- | | Mouse | | 100 V, | and 2nd Abs, 5 min each |
| | milk | milk | | 293182 | | IgG, | | 120 min | time |
| | | | | | | 1:4000 | | | |
| FN1 | 5% | 5% | 1:1000 | abcam ab | 5% milk | Anti- | | 0.45 um | Wash 3 times after 1st |
| | nonfat | nonfat | | 194395 | | Mouse | | 100 V, | and 2nd Abs, 5 min each |
| | milk | milk | | | | IgG | | 120 min | time |
| | | | | | | 1:4000 | | | |
| TSP1 | 5% | 5% BSA | 1:500 | abcam ab | 5% milk | Anti- | | 0.45 um | Wash 3 times after 1st |
| | nonfat | | | 85762 | | Rabbit | | 100 V, | and 2nd Abs, 5 min each |
| | milk | | | | | IgG | | 120 min | time |
| | | | | | | 1:4000 | | | |
| TGFbR2 | 5% | 5% | 1:1000 | abcam ab | 5% milk | Anti- | | 0.2 um | Wash 3 times after 1st |
| | nonfat | nonfat | | 186838 | | Rabbit | | 100 V, | and 2nd Abs, 5 min each |
| | milk | milk | | | | IgG | | 30 min | time |
| | | | | | | 1:5000 | | | |
| ACTA2 | 5% | 5% | 1:1000 | abcam ab | 5% milk | Anti- | | 0.2 um | Wash 3 times after 1st |
| | nonfat | nonfat | | 293182 | | Mouse | | 100 V, | and 2nd Abs, 5 min each |
| | milk | milk | | | | IgG | | 30 min | time |
| | | | | | | 1:5000 | | | |
| GAPDH | 5% | 5% | 1:10000 | abcam ab | 5% milk | Anti- | | 0.2 um | Wash 3 times after 1st |
| | nonfat | nonfat | | 128915 | | Rabbit | | 100 V, | and 2nd Abs, 5 min each |
| | milk | milk | | | | IgG | | 30 min | time |
| | | | | | | 1:5000 | | | |
| TGFb1 | 5% | 5% | 1:250 | abcam ab | 5% milk | Anti- | | 0.2 um | Wash 3 times after 1st |
| | nonfat | nonfat | | 92486 | | Rabbit | | 100 V, | and 2nd Abs, 5 min each |
| | milk | milk | | | | IgG | | 30 min | time |
| | | | | | | 1:4000 | | | |
| Lin28B | 5% | 5% | 1:1000 | Thermo | 5% milk | Anti- | | 0.2 um | Wash 3 times after 1st |
| | nonfat | nonfat | | Fisher | | Rabbit | | 100 V, | and 2nd Abs, 5 min each |
| | milk | milk | | PA5- | | IgG | | 30 min | time |
| | | | | 50609 | | 1:4000 | | | |
| Samd3 | 5% | 5% | 1:1000 | cell | 5% milk | Anti- | | 0.2 um | Wash 3 times after 1st |
| | nonfat | nonfat | | signaling | | Rabbit | | 100 V, | and 2nd Abs, 5 min each |
| | milk | milk | | C67H9 | | IgG | | 30 min | time |
| | | | | | | 1:4000 | | | |
| p-Samd3 | 5% | 5% BSA | 1:1000 | cell | 5% milk | Anti- | | 0.2 um | Wash 3 times after 1st |
| | nonfat | | | signaling | | Rabbit | | 100 V, | and 2nd Abs, 5 min each |
| | milk | | | C25A9 | | IgG | | 30 min | time |
| | | | | | | 1:4000 | | | |
| TIMP1 | 5% | 5% | 1:1000 | cell | 5% milk | Anti- | | 0.2 um | Wash 3 times after 1st |
| | nonfat | nonfat | | signaling | | Mouse | | 100 V, | and 2nd Abs, 5 min each |
| | milk | milk | | 8946S | | IgG | | 30 min | time |
| | | | | | | 1:5000 | | | |
| mouse | Blocking | 1st Ab | 1st Ab | 1st Ab | 2nd Ab | 2nd Ab | 2nd Ab | Transfer | |
| liver | 1 h | 4° C. O/N | dilution | Vendor | RT 2 h | dilution | Vendor | on ice | Wash |
| TET3 | 5% | 5% | 1:500 | GENE | 5% milk | Anti- | | 0.45 um | Wash 3 times after 1st |
| | nonfat | nonfat | | Tex | | Rabbit | | 100 V, | and 2nd Abs, 5 min each |
| | milk | milk | | 121453 | | IgG, | | 120 min | time |
| | | | | | | 1:5000 | | | |
| TGFb1 | 5% | 5% | 1:250 | abcam ab | 5% milk | Anti- | | 0.2 um | Wash 3 times after 1st |
| | nonfat | nonfat | | 92486 | | Rabbit | | 100 V, | and 2nd Abs, 5 min each |
| | milk | milk | | | | IgG | | 30 min | time |
| | | | | | | 1:4000 | | | |
| Col1A1 | 5% | 5% | 1:500 | Santa | 5% milk | Anti- | | 0.45 um | Wash 3 times after 1st |
| | nonfat | nonfat | | cruz sc- | | Mouse | | 100 V, | and 2nd Abs, 5 min each |
| | milk | milk | | 293182 | | IgG, | | 120 min | time |
| | | | | | | 1:5000 | | | |
| FN1 | 5% | 5% | 1:1000 | abcam ab | 5% milk | Anti- | | 0.45 um | Wash 3 times after 1st |
| | nonfat | nonfat | | 194395 | | Mouse | | 100 V, | and 2nd Abs, 5 min each |
| | milk | milk | | | | IgG | | 120 min | time |
| | | | | | | 1:5000 | | | |
| ACTA2 | 5% | 5% | 1:1000 | abcam ab | 5% milk | Anti- | | 0.2 um | Wash 3 times after 1st |
| | nonfat | nonfat | | 293182 | | Mouse | | 100 V, | and 2nd Abs, 5 min each |
| | milk | milk | | | | IgG | | 30 min | time |
| | | | | | | 1:5000 | | | |
| TSP1 | 5% | 5% BSA | 1:500 | abcam ab | 5% milk | Anti- | | 0.45 um | Wash 3 times after 1st |
| | nonfat | | | 85762 | | Rabbit | | 100 V, | and 2nd Abs, 5 min each |
| | milk | | | | | IgG | | 120 min | time |
| | | | | | | 1:5000 | | | |
| GAPDH | 5% | 5% | 1:1000 | cell | 5% milk | Anti- | | 0.2 um | Wash 3 times after 1st |
| | nonfat | nonfat | | signaling | | Rabbit | | 100 V, | and 2nd Abs, 5 min each |
| | milk | milk | | 2118L | | IgG | | 30 min | time |
| | | | | | | 1:5000 | | | |
| TGFbR2 | 5% | 5% | 1:1000 | abcam ab | 5% milk | Anti- | | 0.2 um | Wash 3 times after 1st |
| | nonfat | nonfat | | 186838 | | Rabbit | | 100 V, | and 2nd Abs, 5 min each |
| | milk | milk | | | | IgG | | 30 min | time |
| | | | | | | 1:5000 | | | |
| TIMP1 | 5% | 5% | 1:1000 | cell | 5% milk | Anti- | | 0.2 um | Wash 3 times after 1st |
| | nonfat | nonfat | | signaling | | Mouse | | 100 V, | and 2nd Abs, 5 min each |
| | milk | milk | | 8946S | | IgG | | 30 min | time |
| | | | | | | 1:5000 | | | |

Genomic DNA Extraction

Genomic DNA (gDNA) was isolated from LX-2 cells grown in 24-well plates using Quick-gDNA MicroPrep (Zymo, D3021) according to the manufacturer's instructions.

Immunohistochemistry (IHC)

Paraffin sections were used to perform IHC using the EnVision G2 Doublestain System (K5361, DAKO, DAB+/Permanent Red). Briefly, paraffin slides were dewaxed in p-xylene (Sigma, 185566-1L) overnight, followed by sequential washes in 100%, 90%, 80%, and 70% ethanol and two times of ddH2O for 5 min each. At the end of the final wash, slides were immediately placed in boiling sodium citrate buffer (10 mM, pH 6.0) for 20 min for antigen retrieval. After gentle wash for 5 min, 3 times with 1×PBS, 200 μl of Dual Endogenous Enzyme Block was added to cover the specimen and incubation at RT was carried out for 5 min, followed by 2 times of PBS wash, 5 min each. 200 μl of primary antibody (TGF-β1 at 1:50 dilution, ab 92486; TET3 at 1:50 dilution, GENE Tex, 121453) or negative control IgG was added and incubation was carried out at RT for 1 h. The slides were washed for 5 min, 3 times with PBS. 200 μl of polymer/HRP was added for 1 h, followed by addition of DAB working solution (1:50 DAB chromogen/DAB buffer) and incubation for 5-15 min. Subsequently, the slides were sequentially incubated with double block for 3 min, primary antibody against α-SMA (diluted at 1:400, ab 293182), Mouse/Rabbit (Link), and polymer/AP for 30 min, and permanent red working solution for 5-20 min. Next, slides were incubated with hematoxylin for 1 min and then 0.037 mmol/L NH₄OH for 2-5 min, followed by wash twice with ddH₂O. After that, the slides were washed with 70%, 80%, 90% ethanol, and 100% ethanol and p-xylene for 5 min each. After air dry, the slides were mounted with aqueous mounting media (Immu-Mount from Thermo). IHC images were captured using confocal microscopy (Leica SP5).

Hydroxyproline and Blood Chemistry

Liver tissue hydroxyproline content was assessed using Hydroxyproline Assay Kit (Sigma-Aldrich, MAK008-1KT) according to the manufacture's protocol. Serum bilirubin concentration was determined in a 96-well plate scale using Bilirubin Assay kit (Sigma-Aldrich, MAK126) according to the manufacture's protocol. Briefly, 50 μl of Calibrator and 50 mL of ddH2O were transferred into two separate wells in a 96-well plate, followed by addition of 200 μl of ddH2O into each well for a final volume of 250 μl. Then, 50 μl of serum sample was added to each well, followed by addition of freshly prepared 200 μl of Working Reagent and incubation at RT for 10 min. Absorbance was measured at 530 nm (A530) in a multi-model plate reader (Molecular Device, Filter Max F5). Bilirubin concentration was calculated using the following equation:

Bilirubin concentration={[(A530)sample−(A530)blank]/[(A530)calibrator−(A530)water]}×(5 mg/dL)

Serum ALP and ALT were measured by the Department of Laboratory Medicine, Yale-New Haven Hospital.

Let-7 Binding Sites Prediction

The RNAhybrid program (Kruger et al., 2006, Nucleic Acids Res 34:W451-454) (https://bibiserv2.cebitec.uni-bielefeld.de/rnahybrid?id=rnahybrid_view_submission) was used to predict let-7 binding sites in TET1 mRNA.

Statistical Analysis

The number of independent experiments and the statistical analysis for each figure are indicated in the legends. All statistical analyses were performed using GraphPad Prism version 7.01 for Windows (GraphPad Software, La Jolla Calif. USA, www.graphpad.com) and are presented as mean±SEM. Two-tailed Student's t tests (or as otherwise indicated) were used to compare means between groups. p<0.05 was considered significant. Key reagents and chemicals referred to Table 2.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of preventing or treating a disease or disorder associated with increased ten-eleven translocation protein (TET) level, increased H19 level, an increased level of transforming growth factor (TGF) signaling, or any combination thereof;
the method comprising administering a treatment to a subject in need thereof,
wherein the treatment comprises at least one selected from the group consisting of a reduction of TET level or activity, a reduction of H19 level or activity, a reduction of TGF signaling or activity, a reduction of hepatocyte nuclear factor (HNF) level or activity, a reduction of hepatocyte nuclear factor (HNF) isoform level or activity, and any combination thereof, in a subject in need thereof.

2. The method of claim 1, wherein the ten-eleven translocation protein (TET) is selected from the group consisting of a ten-eleven translocation protein 1 (TET1), ten-eleven translocation protein 2 (TET2), ten-eleven translocation protein 3 (TET3), and any combination thereof.

3. The method of claim 1, wherein the transforming growth factor (TGF) signaling comprises at least one component selected from the group consisting of a transforming growth factor alpha (TGF-α), transforming growth factor alpha (TGF-α) isoform, transforming growth factor beta (TGF-β), transforming growth factor beta (TGF-β)isoform, TGF-β receptor 1 (TGFBR1), TGF-β receptor 2 (TGFBR2), Smad protein 2 (Smad2), Smad protein 3 (Smad3), Smad protein 4 (Smad4), phosphorylated SMAD2 (p-SMAD2), phosphorylated SMAD3 (p-SMAD3), phosphorylated SMAD4 (p-SMAD4), and any combination thereof.

4. (canceled)

5. The method of claim 1, wherein the hepatocyte nuclear factor (HNF) is selected from the group consisting of a hepatocyte nuclear factor 1 alpha (HNF1α), hepatocyte nuclear factor 1 beta (HNF1β), hepatocyte nuclear factor 3 alpha (HNF3α), hepatocyte nuclear factor 3 beta (HNF3β), hepatocyte nuclear factor 4 alpha (HNF4α), hepatocyte nuclear factor 4 gamma (HNF4γ), hepatocyte nuclear factor 6 alpha (HNF6α), hepatocyte nuclear factor 6 beta (HNF6β), and any combination thereof.

6.-8. (canceled)

9. The method of claim 1, wherein the method of treatment comprises administering a therapeutically effective amount of at least one selected from the group consisting of a TET inhibitor, a H19 inhibitor, a TGF signaling inhibitor, a HNF inhibitor, and any combination thereof to a subject in need thereof.

10. The method of claim 9, wherein the TET inhibitor is a TET1 inhibitor, a TET2 inhibitor, a TET3 inhibitor, or any combination thereof.

11.-12. (canceled)

13. The method of claim 9, wherein the TGF inhibitor is a TGFα inhibitor, a TGFβ1 inhibitor, a TGFβ2 inhibitor, a TGFβ3 inhibitor, a TGFβ4 inhibitor, a TGFBR1 inhibitor, a TGFBR2 inhibitor, a Smad2 inhibitor, a Smad3 inhibitor, a Smad4 inhibitor, or any combination thereof.

14. (canceled)

15. The method of claim 9, wherein the HNF inhibitor is a HNF1α inhibitor, a HNF1β inhibitor, a HNF3α inhibitor, a HNF3β inhibitor, a HNF4α inhibitor, a HNF4γ inhibitor, a HN6α inhibitor, a HNF6β inhibitor, or any combination thereof.

16. (canceled)

17. The method of claim 1, wherein the reduction of TET level or activity comprises an inhibition of TET.

18. The method of claim 1, wherein the reduction of TET level or activity comprises a reduction of at least one TET co-factor or a reduction of H19 level or activity.

19. The method of claim 18, wherein the TET co-factor is selected from the group consisting of α-ketoglutarate, vitamin C, iron, 2-oxoglutarate, and any combination thereof.

20. The method of claim 1, wherein the reduction of TET level or activity comprises a siRNA knockdown of TET.

21. The method of claim 20, wherein the siRNA knockdown of TET is a viral-mediated siRNA knockdown of TET.

22. The method of claim 21, wherein the viral-mediated siRNA knockdown of TET comprises at least one AAV vector.

23.-25. (canceled)

26. The method of claim 1, wherein the treatment further comprises a reduction of at least one selected from the group consisting of estradiol, progesterone, glucagon, MED12, GRAF1, SPARC, PEPCK, G6PC, PGC, PGC-1α, TSP, TSP1, FN1, VIM, COL1A1, COL3A1, COL4A1, COL5A2, HMGA2, SLUG, T1MP1, α-SMA, SMAD2, SMAD3, SMAD4, p-SMAD2, p-SMAD3, p-SMAD4, SM22-α, LIN28B, NOTCH3, collagen 1, fibronectin, and any combination thereof, in a subject in need thereof.

27. The method of claim 1, wherein the treatment further comprises reducing an expression of at least one selected from the group consisting of Teti, Hnf4α, Pck1, G6pc, and any combination thereof, in a subject in need thereof.

28. The method of claim 1, wherein the disease or disorder is a disease or disorder associated with gluconeogenesis regulation.

29. The method of claim 1, wherein the disease or disorder is a cancer or fibrosis.

30.-32. (canceled)

33. The method of claim 1, wherein the method of treatment comprises administering a therapeutically effective amount of at least one FOXA2 inhibitor.

34. (canceled)

35. The method of claim 1, wherein the method of treatment comprises administering a therapeutically effective amount of at least one let-7 promoter.

36. (canceled)
